(19) United States
(12) Patent Application Publication
(10) Pub. No.: US 2026/0158142 A1
(43) Pub. Date: Jun. 11, 2026

(54) CELL THERAPY FOR ALZHEIMER'S DISEASE

(71) Applicant: Board of Regents of the University Of Nebraska, Lincoln, NE (US)

(72) Inventors: Howard GENDELMAN, Omaha, NE (US); R. Lee MOSLEY, Omaha, NE (US); Pravin YEAPURI, Omaha, NE (US); Jatinkumar MACHHI, Omaha, NE (US)

(73) Assignee: Board of Regents of the University Of Nebraska, Lincoln, NE (US)

(21) Appl. No.: 18/856,470

(22) PCT Filed: Feb. 13, 2023
(86) PCT No.: PCT/US2023/062514

§ 371 Date: Oct. 11, 2024

Related U.S. Application Data
(60) Provisional application No. 63/330,830, filed on Apr. 14, 2022.

(51) Int. Cl. A61K 40/31; A61K 35/17; A61K 38/46; A61K 40/11; A61K 40/41; A61P 25/28; C07K 14/725; C07K 16/18; C12N 5/0783; C12N 9/22; C12N 15/11; C12N 15/86; G01N 33/68

(52) U.S. Cl. CPC A61K 40/31; A61K 35/17; A61K 38/465; A61K 40/11; A61K 40/414; A61P 25/28; C07K 14/7051; C07K 16/18; C12N 5/0636; C12N 9/226; C12N 15/111; C12N 15/86; G01N 33/6896; A61K 2239/13; C07K 2317/24; C07K 2317/33; C07K 2317/565; C12N 2310/20; C12N 2510/00; C12N 2750/14143; G01N 2333/4709

(57) ABSTRACT

Provided are engineered cells that include a T cell receptor (TCR) or antigen-binding fragment thereof that binds to amyloid beta, and methods of engineering and using such cells, such as in methods of treatment, diagnosis, and monitoring of therapeutic effectiveness, of diseases or conditions, such as those associated with amyloid beta, e.g., Alzheimer's Disease.

### CROSS-REFERENCE TO RELATED APPLICATIONS

This application claims priority to U.S. Provisional Patent Application No. 63/330,830, filed Apr. 14, 2022, entitled “Cell Therapy for Alzheimer's Disease,” the contents of which is hereby incorporated by reference in its entirety for all purposes.

### STATEMENT REGARDING FEDERALLY SPONSORED RESEARCH OR DEVELOPMENT

This invention was made with government support under Grants Nos. P01 DA028555, R01 NS036126, P01 NS031492, P01 MH064570, P01 NS043985, P30 MH062261, R01 AG043540, and R01 NS034239, all awarded by the National Institutes of Health. The government has certain rights in the invention.

### INCORPORATION BY REFERENCE OF SEQUENCE LISTING

The present application is being filed along with a Sequence Listing in electronic format. The Sequence Listing is provided as a file entitled 166982000240SeqList.xml, created Feb. 9, 2023, which is 30,764 bytes in size. The information in the electronic format of the Sequence Listing is incorporated by reference in its entirety.

### FIELD

The present disclosure relates in some aspects to engineered cells that include a T cell receptor (TCR) or antigen-binding fragment thereof that binds to amyloid beta, and methods of engineering and using such cells, e.g., in methods of treatment, diagnosis, and monitoring of therapeutic effectiveness.

### BACKGROUND

Alzheimer's disease (AD) is the most common age-related dementia. Nationwide, the number of persons living with AD and the cost of delivered care is rising exponentially. Alzheimer's disease is now the sixth leading cause of death in the United States. From 2000 to 2015, Alzheimer's disease-associated mortalities have increased by 123%. More problematic is that Alzheimer's disease cannot currently be prevented, cured, or slowed. Existing small molecule therapies can provide only symptomatic relief and since the approval of the last Alzheimer's disease medicine in 2003, more than 400 drug candidates have failed in the clinical testing. Accordingly, there is a need in the art for new therapies to treat AD.

### I. SUMMARY

In some aspects, provided herein is an engineered cell, comprising a heterologous T cell receptor (TCR) or antigen-binding fragment thereof, wherein the heterologous TCR or antigen-binding fragment thereof comprises an alpha chain comprising a variable alpha (Vα) region and a beta chain comprising a variable beta (Vβ) region, and wherein the Vα region comprises a complementarity determining region 1 (CDR-1), CDR-2, and CDR-3 comprising the amino acid sequences of SEQ ID NOs: 12, 13, and 14, respectively; and the Vβ region comprises a CDR-1, CDR-2, and CDR-3 comprising the amino acid sequences of SEQ ID NOs: 15, 16, and 17, respectively.

In some aspects, provided herein is an engineered cell, comprising a heterologous T cell receptor (TCR) or antigen-binding fragment thereof, wherein the heterologous TCR or antigen-binding fragment thereof comprises an alpha chain comprising a variable alpha (Vα) region and a beta chain comprising a variable beta (Vβ) region, and wherein the Vα region comprises a complementarity determining region 1 (CDR-1), CDR-2, and CDR-3 comprising the CDR-1, CDR-2, and CDR-3 amino acid sequences contained within the amino acid sequence of SEQ ID NO: 2 or 5; and the Vβ region comprises a CDR-1, CDR-2, and CDR-3 comprising the CDR-1, CDR-2, and CDR-3 amino acid sequences contained within the amino acid sequence of SEQ ID NO: 4 or 8.

In some of any of the provided embodiments, the engineered cell further comprises a genetic disruption in one or more genes endogenous to the cell. In some of any of the provided embodiments, the one or more genes endogenous to the cell comprises a T Cell Receptor Alpha Constant (TRAC) gene and/or a T Cell Receptor Beta Constant (TRBC) gene. In some of any of the provided embodiments, the engineered cell is a T cell, a B cell, or a Natural Killer (NK) cell. In some of any of the provided embodiments, the engineered cell is a T cell. In some of any of the provided embodiments, the T cell is a regulatory T cell or an effector T cell. In some of any of the provided embodiments, the T cell is a regulatory T (Treg) cell. In some of any of the provided embodiments, the engineered cell further comprises a nucleic acid molecule encoding the TCR or antigen-binding fragment thereof. In some of any of the provided embodiments, the nucleic acid molecule is under the control of one or more endogenous gene promoters. In some of any of the provided embodiments, the one or more endogenous gene promoters comprises the endogenous TRAC gene promoter and/or the endogenous TRBC gene promoter. In some of any of the provided embodiments, the nucleic acid molecule is under the control of an exogenous promoter. In some of any of the provided embodiments, the nucleic acid molecule is inserted into the endogenous TRAC gene and/or the endogenous TRBC gene.

In some aspects, provided herein is an engineered cell, comprising: a heterologous T cell receptor (TCR) or antigen-binding fragment thereof, wherein the heterologous TCR or antigen-binding fragment thereof comprises an alpha chain comprising a variable alpha (Vα) region and a beta chain comprising a variable beta (Vβ) region, and wherein the Vα region comprises a complementarity determining region 1 (CDR-1), CDR-2, and CDR-3 comprising the amino acid sequences of SEQ ID NOs: 12, 13, and 14, respectively; and the Vβ region comprises a CDR-1, CDR-2, and CDR-3 comprising the amino acid sequences of SEQ ID NOs: 15, 16, and 17, respectively; and a genetic disruption in a T Cell Receptor Alpha Constant (TRAC) gene and/or a T Cell Receptor Beta Constant (TRBC) gene.

In some aspects, provided herein is an engineered cell, comprising: a heterologous T cell receptor (TCR) or antigen-binding fragment thereof, wherein the heterologous TCR or antigen-binding fragment thereof comprises an alpha chain comprising a variable alpha (Vα) region and a beta chain comprising a variable beta (Vβ) region, and wherein the Vα region comprises a complementarity determining region 1 (CDR-1), CDR-2, and CDR-3 comprising the CDR-1, CDR-2, and CDR-3 amino acid sequences contained within the amino acid sequence of SEQ ID NO: 2 or 5; and the Vβ region comprises a CDR-1, CDR-2, and CDR-3 comprising the CDR-1, CDR-2, and CDR-3 amino acid sequences contained within the amino acid sequence of SEQ ID NO: 4 or 8; and a genetic disruption in a T Cell Receptor Alpha Constant (TRAC) gene and/or a T Cell Receptor Beta Constant (TRBC) gene.

In some aspects, provided herein is an engineered regulatory T (Treg) cell, comprising: a heterologous T cell receptor (TCR) or antigen-binding fragment thereof, wherein the heterologous TCR or antigen-binding fragment thereof comprises an alpha chain comprising a variable alpha (Vα) region and a beta chain comprising a variable beta (Vβ) region, and wherein the Vα region comprises a complementarity determining region 1 (CDR-1), CDR-2, and CDR-3 comprising the amino acid sequences of SEQ ID NOs: 12, 13, and 14, respectively; and the Vβ region comprises a CDR-1, CDR-2, and CDR-3 comprising the amino acid sequences of SEQ ID NOs: 15, 16, and 17, respectively; and a genetic disruption in a T Cell Receptor Alpha Constant (TRAC) gene and/or a T Cell Receptor Beta Constant (TRBC) gene.

In some aspects, provided herein is an engineered regulatory T (Treg) cell, comprising: a heterologous T cell receptor (TCR) or antigen-binding fragment thereof, wherein the heterologous TCR or antigen-binding fragment thereof comprises an alpha chain comprising a variable alpha (Vα) region and a beta chain comprising a variable beta (Vβ) region, and wherein the Vα region comprises a complementarity determining region 1 (CDR-1), CDR-2, and CDR-3 comprising the CDR-1, CDR-2, and CDR-3 amino acid sequences contained within the amino acid sequence of SEQ ID NO: 2 or 5; and the Vβ region comprises a CDR-1, CDR-2, and CDR-3 comprising the CDR-1, CDR-2, and CDR-3 amino acid sequences contained within the amino acid sequence of SEQ ID NO: 4 or 8; and a genetic disruption in a T Cell Receptor Alpha Constant (TRAC) gene and/or a T Cell Receptor Beta Constant (TRBC) gene.

In some aspects, provided herein is an engineered cell, comprising: a nucleic molecule encoding a heterologous T cell receptor (TCR) or antigen-binding fragment thereof, wherein the heterologous TCR or antigen-binding fragment thereof comprises an alpha chain comprising a variable alpha (Vα) region and a beta chain comprising a variable beta (Vβ) region, and wherein the Vα region comprises a complementarity determining region 1 (CDR-1), CDR-2, and CDR-3 comprising the amino acid sequences of SEQ ID NOs: 12, 13, and 14, respectively; and the Vβ region comprises a CDR-1, CDR-2, and CDR-3 comprising the amino acid sequences of SEQ ID NOs: 15, 16, and 17, respectively; and a genetic disruption in a T Cell Receptor Alpha Constant (TRAC) gene and/or a T Cell Receptor Beta Constant (TRBC) gene.

In some aspects, provided herein is an engineered cell, comprising: a nucleic acid molecule encoding a heterologous T cell receptor (TCR) or antigen-binding fragment thereof, wherein the heterologous TCR or antigen-binding fragment thereof comprises an alpha chain comprising a variable alpha (Vα) region and a beta chain comprising a variable beta (Vβ) region, and wherein the Vα region comprises a complementarity determining region 1 (CDR-1), CDR-2, and CDR-3 comprising the CDR-1, CDR-2, and CDR-3 amino acid sequences contained within the amino acid sequence of SEQ ID NO: 2 or 5; and the Vβ region comprises a CDR-1, CDR-2, and CDR-3 comprising the CDR-1, CDR-2, and CDR-3 amino acid sequences contained within the amino acid sequence of SEQ ID NO: 4 or 8; and a genetic disruption in a T Cell Receptor Alpha Constant (TRAC) gene and/or a T Cell Receptor Beta Constant (TRBC) gene. In some of any of the provided embodiments, the engineered cell further comprises a nucleic acid molecule encoding the TCR or antigen-binding fragment thereof. In some of any of the provided embodiments, the nucleic acid molecule is under the control of one or more endogenous gene promoters. In some of any of the provided embodiments, the one or more endogenous gene promoters comprises the endogenous TRAC gene promoter and/or the endogenous TRBC gene promoter. In some of any of the provided embodiments, the nucleic acid molecule is under the control of an exogenous promoter. In some of any of the provided embodiments, the nucleic acid molecule is inserted into the endogenous TRAC gene and/or the endogenous TRBC gene. In some of any of the provided embodiments, the nucleic acid molecule is under the control of the endogenous TRAC gene promoter and/or the endogenous TRBC gene promoter. In some of any of the provided embodiments, the genetic disruption results in a reduction or elimination of expression of the gene product of the endogenous gene.

In some of any of the provided embodiments, the Vα region comprises a CDR-1, CDR-2, and CDR-3 comprising the amino acid sequences of SEQ ID NOs: 12, 13, and 14, respectively; and the Vβ region comprises a CDR-1, CDR-2, and CDR-3 comprising the amino acid sequences of SEQ ID NOs: 15, 16, and 17, respectively; or the Vα region comprises a CDR-1, CDR-2, and CDR-3 comprising the CDR-1, CDR-2, and CDR-3 amino acid sequences contained within the amino acid sequence of SEQ ID NO: 2 or 5; and the Vβ region comprises a CDR-1, CDR-2, and CDR-3 comprising the CDR-1, CDR-2, and CDR-3 amino acid sequences contained within the amino acid sequence of SEQ ID NO: 4 or 8. In some of any of the provided embodiments, the Vα region comprises the amino acid sequence of SEQ ID NO: 5 or an amino acid sequence having at least 80%, 85%, 90%, 91%, 92%, 93%, 94%, 95%, 96%, 97%, 98%, or 99% identity to SEQ ID NO: 5; and/or the Vβ comprises the amino acid sequence of SEQ ID NO: 8 or an amino acid sequence having at least 80%, 85%, 90%, 91%, 92%, 93%, 94%, 95%, 96%, 97%, 98%, or 99% identity to the amino acid sequence of SEQ ID NO: 8. In some of any of the provided embodiments, the Vα region comprises an amino acid sequence of SEQ ID NO: 5 and/or the Vβ region comprises the amino acid sequence of SEQ ID NO: 8. In some of any of the provided embodiments, the Vα region comprises an amino acid sequence of SEQ ID NO: 5 and the Vβ region comprises the amino acid sequence of SEQ ID NO: 8. In some of any of the provided embodiments, the TCR or antigen-binding fragment thereof specifically binds to human amyloid beta. In some of any of the provided embodiments, the alpha chain further comprises an alpha constant (Cα) region and/or the beta chain further comprises a beta constant (Cβ) region. In some of any of the provided embodiments, the Cα region comprises the amino acid sequence of SEQ ID NO: 7, or an amino acid sequence having at least 80%, 85%, 86%, 87%, 88%, 89%, 90%, 91%, 92%, 93%, 94%, 95%, 96%, 97%, 98%, or 99% sequence identity to the amino acid sequence of SEQ ID NO: 7; and/or the Cβ region comprises the amino acid sequence of SEQ ID NO: 11, or an amino acid sequence having at least 80%, 85%, 86%, 87%, 88%, 89%, 90%, 91%, 92%, 93%, 94%, 95%, 96%, 97%, 98%, or 99% sequence identity to the amino acid sequence of SEQ ID NO: 11. In some of any of the provided embodiments, the Cα region comprises the amino acid sequence of SEQ ID NO: 7, or an amino acid sequence having at least 80%, 85%, 86%, 87%, 88%, 89%, 90%, 91%, 92%, 93%, 94%, 95%, 96%, 97%, 98%, or 99% sequence identity to the amino acid sequence of SEQ ID NO: 7; and the Cβ region comprises the amino acid sequence of SEQ ID NO: 11, or an amino acid sequence having at least 80%, 85%, 86%, 87%, 88%, 89%, 90%, 91%, 92%, 93%, 94%, 95%, 96%, 97%, 98%, or 99% sequence identity to the amino acid sequence of SEQ ID NO: 11. In some of any of the provided embodiments, the Cα region comprises the amino acid sequence of SEQ ID NO: 7; and/or the Cβ region comprises the amino acid sequence of SEQ ID NO: 11. In some of any of the provided embodiments, the alpha chain comprises the amino acid sequence of SEQ ID NO: 2 or an amino acid sequence having at least 80%, 85%, 90%, or 95% identity to SEQ ID NO: 2; and/or the beta chain comprises the amino acid sequence of SEQ ID NO: 4 or an amino acid sequence having at least 80%, 85%, 90%, or 95% identity to SEQ ID NO: 4.

In some of any of the provided embodiments, the engineered cell is chimeric. In some of any of the provided embodiments, the engineered cell is humanized. In some of any of the provided embodiments, the engineered cell is murine. In some of any of the provided embodiments, the engineered cell is a T cell, a B cell, or a Natural Killer (NK) cell. In some of any of the provided embodiments, the engineered cell is a T cell. In some of any of the provided embodiments, the T cell is a regulatory T cell or an effector T cell. In some of any of the provided embodiments, the T cell is a regulatory T (Treg) cell.

In some aspects, provided herein is a T cell receptor (TCR) or antigen-binding fragment thereof, comprising an alpha chain comprising a variable alpha (Vα) region and a beta chain comprising a variable beta (Vβ) region, wherein the Vα region comprises a complementarity determining region 1 (CDR-1), CDR-2, and CDR-3 comprising the amino acid sequences of SEQ ID NOs: 12, 13, and 14, respectively; and the Vβ region comprises a CDR-1, CDR-2, and CDR-3 comprising the amino acid sequences of SEQ ID NOs: 15, 16, and 17, respectively. In some aspects, provided herein is a T cell receptor (TCR) or antigen-binding fragment thereof, comprising an alpha chain comprising a variable alpha (Vα) region and a beta chain comprising a variable beta (Vβ) region, wherein the Vα region comprises a complementarity determining region 1 (CDR-1), CDR-2, and CDR-3 comprising the CDR-1, CDR-2, and CDR-3 amino acid sequences contained within the amino acid sequence of SEQ ID NO: 2 or 5; and the Vβ region comprises a CDR-1, CDR-2, and CDR-3 comprising the CDR-1, CDR-2, and CDR-3 amino acid sequences contained within the amino acid sequence of SEQ ID NO: 4 or 8. In some aspects, provided herein is a TCR or antigen-binding fragment thereof of claim 37 or claim 38, wherein the Vα region comprises a CDR-1, CDR-2, and CDR-3 comprising the CDR-1, CDR-2, and CDR-3 amino acid sequences contained within the amino acid sequence of SEQ ID NO: 2; and the Vβ region comprises a CDR-1, CDR-2, and CDR-3 comprising the CDR-1, CDR-2, and CDR-3 amino acid sequences contained within the amino acid sequence of SEQ ID NO: 4.

In some of any of the provided embodiments, the Vα region comprises a CDR-1, CDR-2, and CDR-3 comprising the CDR-1, CDR-2, and CDR-3 amino acid sequences contained within the amino acid sequence of SEQ ID NO: 5; and the Vβ region comprises a CDR-1, CDR-2, and CDR-3 comprising the CDR-1, CDR-2, and CDR-3 amino acid sequences contained within the amino acid sequence of SEQ ID NO: 8. In some of any of the provided embodiments, the Vα region comprises the amino acid sequence of SEQ ID NO: 5 or an amino acid sequence having at least 80%, 85%, 90%, 91%, 92%, 93%, 94%, 95%, 96%, 97%, 98%, or 99% identity to SEQ ID NO: 5; and/or wherein the Vβ comprises the amino acid sequence of SEQ ID NO: 8 or an amino acid sequence having at least 80%, 85%, 90%, 91%, 92%, 93%, 94%, 95%, 96%, 97%, 98%, or 99% identity to the amino acid sequence of SEQ ID NO: 8. In some of any of the provided embodiments, the Vα region comprises an amino acid sequence of SEQ ID NO: 5 and/or the Vβ region comprises the amino acid sequence of SEQ ID NO: 8. In some of any of the provided embodiments, the Vα region comprises an amino acid sequence of SEQ ID NO: 5 and the Vβ region comprises the amino acid sequence of SEQ ID NO: 8. In some of any of the provided embodiments, the TCR or antigen-binding fragment thereof specifically binds to amyloid beta. In some of any of the provided embodiments, the TCR or antigen-binding fragment thereof specifically binds to human amyloid beta. In some of any of the provided embodiments, the alpha chain further comprises an alpha constant (Cα) region and/or the beta chain further comprises a beta constant (Cβ) region. In some of any of the provided embodiments, the Cα region comprises the amino acid sequence of SEQ ID NO: 7, or an amino acid sequence having at least 80%, 85%, 86%, 87%, 88%, 89%, 90%, 91%, 92%, 93%, 94%, 95%, 96%, 97%, 98%, or 99% sequence identity to the amino acid sequence of SEQ ID NO: 7; and/or the Cβ region comprises the amino acid sequence of SEQ ID NO: 11, or an amino acid sequence having at least 80%, 85%, 86%, 87%, 88%, 89%, 90%, 91%, 92%, 93%, 94%, 95%, 96%, 97%, 98%, or 99% sequence identity to the amino acid sequence of SEQ ID NO: 11. In some of any of the provided embodiments, the Cα region comprises the amino acid sequence of SEQ ID NO: 7, or an amino acid sequence having at least 80%, 85%, 86%, 87%, 88%, 89%, 90%, 91%, 92%, 93%, 94%, 95%, 96%, 97%, 98%, or 99% sequence identity to the amino acid sequence of SEQ ID NO: 7; and the Cβ region comprises the amino acid sequence of SEQ ID NO: 11, or an amino acid sequence having at least 80%, 85%, 86%, 87%, 88%, 89%, 90%, 91%, 92%, 93%, 94%, 95%, 96%, 97%, 98%, or 99% sequence identity to the amino acid sequence of SEQ ID NO: 11. In some of any of the provided embodiments, the Cα region comprises the amino acid sequence of SEQ ID NO: 7; and/or the Cβ region comprises the amino acid sequence of SEQ ID NO: 11. In some of any of the provided embodiments, the alpha chain comprises the amino acid sequence of SEQ ID NO: 2 or an amino acid sequence having at least 80%, 85%, 90%, or 95% identity to SEQ ID NO: 2; and/or the beta chain comprises the amino acid sequence of SEQ ID NO: 4 or an amino acid sequence having at least 80%, 85%, 90%, or 95% identity to SEQ ID NO: 4. In some of any of the provided embodiments, the TCR or antigen-binding fragment thereof is chimeric. In some of any of the provided embodiments, the TCR or antigen-binding fragment thereof is humanized. In some aspects, provided herein is a nucleic acid molecule encoding any TCR or antigen-binding fragment thereof, or any alpha or beta chain thereof, or any Vα region or Vβ region thereof provided herein. In some of any of the provided embodiments, the nucleic acid molecule is isolated. In some of any of the provided embodiments, the nucleic acid molecule is codon-optimized. In some of any of the provided embodiments, the nucleic acid molecule is not codon-optimized. In some of any of the provided embodiments, the nucleic acid molecule is DNA.

In some of any of the provided embodiments, the DNA is cDNA. In some of any of the provided embodiments, the nucleic acid molecule is RNA. In some of any of the provided embodiments, the TCR or antigen-binding fragment thereof that is encoded is humanized or chimeric. In some of any of the provided embodiments, the TCR or antigen-binding fragment thereof that is encoded is murine. In some of any of the provided embodiments, the nucleic acid molecule comprises the nucleic acid sequence of SEQ ID NO: 1, or a nucleic acid sequence having at least 80%, 85%, 90%, 91%, 92%, 93%, 94%, 95%, 96%, 97%, 98%, or 99% sequence identity to the nucleic acid sequence of SEQ ID NO: 1; and/or the nucleic acid sequence of SEQ ID NO: 3, or a nucleic acid sequence having at least 80%, 85%, 90%, 91%, 92%, 93%, 94%, 95%, 96%, 97%, 98%, or 99% sequence identity to the nucleic acid sequence of SEQ ID NO: 3.

In some aspects, provided herein is a vector comprising any nucleic acid molecule provided herein. In some aspects, provided herein is a vector comprising any nucleic acid molecule encoding the TCR or antigen-binding fragment thereof, or any alpha and/or beta chain thereof, or any Vα region and/or Vβ region thereof provided herein. In some of any of the provided embodiments, the vector is an expression vector. In some of any of the provided embodiments, the vector is a viral vector. In some of any of the provided embodiments, the viral vector is selected from the group consisting of a retroviral vector, a gammaretroviral vector, a lentiviral vector, and an adeno-associated viral (AAV) vector. In some of any of the provided embodiments, the AAV vector is a self-complementary AAV (scAAV) vector. In some of any of the provided embodiments, the vector is a non-viral vector. In some of any of the provided embodiments, the vector is a donor vector for genome editing. In some of any of the provided embodiments, the vector is a transposon vector. In some of any of the provided embodiments, the transposon vector is a Sleeping Beauty transposon vector or a PiggyBac transposon vector. In some of any of the provided embodiments, the vector is suitable for gene editing or genomic engineering.

In some aspects, provided herein is an engineered cell comprising any nucleic acid molecule or any vector provided herein. In some aspects, provided herein is an engineered cell comprising any TCR or antigen-binding fragment thereof provided herein. In some of any of the provided embodiments, the TCR or antigen-binding fragment thereof is heterologous to the engineered cell. In some aspects, provided herein is a method of producing an engineered cell, comprising introducing any nucleic acid molecule or any vector provided herein, into a cell.

In some aspects, provided herein is a method of producing an engineered cell, comprising introducing any nucleic acid molecule or any vector provided herein, into a cell. In some of any of the provided embodiments, the method is performed in vitro or ex vivo. In some of any of the provided embodiments, the nucleic acid molecule is comprised within a vector.

In some aspects, provided herein is a method of producing a population of engineered cells, comprising introducing any nucleic acid molecule or any vector provided herein, into a cell; and culturing the cell under conditions to produce a population of engineered cells.

In some aspects, provided herein is a method of producing a population of engineered cells, comprising culturing any engineered cell provided herein under conditions to produce a population of engineered cells.

In some aspects, provided herein is a method of engineering a cell, comprising introducing any nucleic acid molecule or any vector provided herein, into a cell.

In some aspects, provided herein is a method of engineering a cell, comprising: introducing any nucleic acid molecule or any vector provided herein, into a cell; and editing and/or disrupting one or more genes endogenous to the cell.

In some of any of the provided embodiments, the introducing is carried out by transfection, electroporation, or transduction. In some of any of the provided embodiments, the introducing is carried out by transfection. In some of any of the provided embodiments, the introducing by transfection comprises introducing any vector provided herein into the cell. In some of any of the provided embodiments, the introducing is carried out by electroporation. In some of any of the provided embodiments, the introducing is carried out by transduction. In some of any of the provided embodiments, the introducing by transduction comprises introducing any vector provided herein into the cell.

In some of any of the provided embodiments, the vector is a viral vector. In some of any of the provided embodiments, the viral vector is selected from the group consisting of a retroviral vector, a gammaretroviral vector, a lentiviral vector, and an adeno-associated viral (AAV) vector. In some of any of the provided embodiments, the AAV vector is a self-complementary AAV (scAAV) vector. In some of any of the provided embodiments, the introducing is carried out using a genome editing technique. In some of any of the provided embodiments, the introducing is carried out using a genome editing technique; and/or the method further comprises a genome editing technique. In some of any of the provided embodiments, the genome editing technique results in editing and/or disrupting one or more genes endogenous to the cell; and/or introducing the nucleic acid molecule encoding the TCR or antigen-binding fragment thereof into a target site. In some of any of the provided embodiments, the target site is comprised within one or more genes endogenous to the cell. In some of any of the provided embodiments, the one or more genes endogenous to the cell comprises a T Cell Receptor Alpha Constant (TRAC) gene and/or a T Cell Receptor Beta Constant (TRBC) gene. In some of any of the provided embodiments, the genome editing technique comprises CRISPR-Cas9 and comprises introducing a crRNA sequence targeting a human TRAC gene that comprises a nucleic acid sequence set forth in SEQ ID NO: 22, and/or comprises introducing a crRNA sequence targeting a human TRBC gene that comprises a nucleic acid sequence set forth in SEQ ID NO: 23.

In some of any of the provided embodiments, the method further comprises introducing into the cell one or more agents capable of editing and/or disrupting one or more genes endogenous to the cell. In some of any of the provided embodiments, the editing and/or disrupting is carried out by one or more agents capable of editing and/or disrupting the one or more genes endogenous to the cell. In some of any of the provided embodiments, the editing and/or disrupting reduces or eliminates expression of the one or more genes endogenous to the cell. In some of any of the provided embodiments, the editing and/or disrupting eliminates expression of the one or more genes endogenous to the cell. In some of any of the provided embodiments, the one or more genes endogenous to the cell each comprise a target site, and one or more of the one or more agents specifically bind to or recognizes the target site. In some of any of the provided embodiments, the one or more genes endogenous to the cell comprises a T Cell Receptor Alpha Constant (TRAC) gene and/or a T Cell Receptor Beta Constant (TRBC) gene. In some of any of the provided embodiments, the one or more agents comprises a nuclease. In some of any of the provided embodiments, the nuclease specifically binds to or recognizes the target site. In some of any of the provided embodiments, the nuclease is selected from the group consisting of a meganuclease, a zinc-finger nuclease, a transcription activator-like effector nuclease (TALEN), a megaTAL nuclease, and a clustered regularly interspaced short palindromic repeats (CRISPR)-associated (Cas) nuclease. In some of any of the provided embodiments, the nuclease is a Cas nuclease. In some of any of the provided embodiments, the Cas nuclease is a Cas9, Cas12a, or Cas13 nuclease. In some of any of the provided embodiments, the Cas nuclease is a Cas9 nuclease. In some of any of the provided embodiments, the method further comprises introducing into the cell one or more agents capable of inserting the nucleic acid molecule into the genome of the cell. In some of any of the provided embodiments, the one or more agents capable of inserting the nucleic acid molecule into the genome of the cell comprises a transposon or a transposon-based system. In some of any of the provided embodiments, the transposon comprises a Sleeping Beauty transposon or a Piggy Bac transposon; or the transposon-based system comprises a Sleeping Beauty transposon-based system or a PiggyBac transposon-based system.

In some aspects, provided herein is a method of engineering a cell, comprising: introducing, into the cell, one or more agents capable of editing and/or disrupting one or more endogenous genes in the cell, wherein each of the one or more endogenous genes comprises a first flanking sequence and a second flanking sequence; and introducing, into the cell, one or more nucleic acid molecules, wherein each of the one or more nucleic acid molecules comprises: (i) a first homology arm and a second homology arm that are homologous to the first flanking sequence and the second flanking sequence of one of the one or more endogenous genes, and (ii) a nucleic acid sequence of interest that is located between the first homology arm and the second homology arm.

In some aspects, provided herein is a method of engineering a cell, comprising: introducing, into the cell, one or more agents capable of inducing a DNA break in one or more endogenous genes in the cell, wherein each of the one or more endogenous genes comprises a first flanking sequence and a second flanking sequence; and introducing, into the cell, one or more nucleic acid molecules, wherein each of the one or more nucleic acid molecules comprises: (i) a first homology arm and a second homology arm that are homologous to the first flanking sequence and the second flanking sequence of one of the one or more endogenous genes, and (ii) a nucleic acid sequence of interest that is located between the first homology arm and the second homology arm.

In some of any of the provided embodiments, the nucleic acid sequence of interest encodes a T cell receptor (TCR) or antigen-binding fragment thereof, wherein the TCR or antigen-binding fragment thereof comprises an alpha chain comprising a variable alpha (Vα) region and a beta chain comprising a variable beta (Vβ) region, wherein the Vα region comprises a complementarity determining region 1 (CDR-1), CDR-2, and CDR-3 comprising the amino acid sequences of SEQ ID NOs: 12, 13, and 14, respectively; and the Vβ region comprises a CDR-1, CDR-2, and CDR-3 comprising the amino acid sequences of SEQ ID NOs: 15, 16, and 17, respectively. In some of any of the provided embodiments, the nucleic acid sequence of interest comprises any nucleic acid molecule provided herein. In some of any of the provided embodiments, the one or more endogenous genes comprises a T Cell Receptor Alpha Constant (TRAC) gene and/or a T Cell Receptor Beta Constant (TRBC) gene.

In some of any of the provided embodiments, the one or more agents capable of editing and/or disrupting one or more endogenous genes in the cell comprises a nuclease. In some of any of the provided embodiments, the one or more agents capable of inducing a DNA break comprises a nuclease. In some of any of the provided embodiments, the nuclease is selected from the group consisting of a meganuclease, a zinc-finger nuclease, a transcription activator-like effector nuclease (TALEN), a megaTAL nuclease, and a clustered regularly interspaced short palindromic repeats (CRISPR)-associated (Cas) nuclease. In some of any of the provided embodiments, the nuclease is a Cas nuclease. In some of any of the provided embodiments, the Cas nuclease is a Cas9, Cas12a, or Cas13 nuclease. In some of any of the provided embodiments, the Cas nuclease is a Cas9 nuclease. In some of any of the provided embodiments, the one or more agents capable of editing and/or disrupting one or more endogenous genes in the cell comprises a Cas nuclease and one or more single guide RNA (sgRNA). In some of any of the provided embodiments, the one or more agents capable of inducing a DNA break comprises a Cas nuclease and one or more single guide RNA (sgRNA). In some of any of the provided embodiments, each of the one or more sgRNA specifically binds to, hybridizes with, or recognizes a target sequence in one of the one or more endogenous genes.

In some of any of the provided embodiments, the one or more sgRNA comprises an sgRNA that specifically binds to, hybridizes with, or recognizes a target sequence in an endogenous TRAC gene, and/or comprises an sgRNA that specifically binds to, hybridizes with, or recognizes a target sequence in an endogenous TRBC gene. In some of any of the provided embodiments, the method results in reduced or eliminated expression of the one or more endogenous genes; and/or introduces expression of the TCR or antigen-binding fragment thereof in the cell. In some of any of the provided embodiments, the method results in reduced or eliminated expression of the one or more endogenous genes and incorporation of a nucleic acid molecule encoding the TCR or antigen-binding fragment thereof into a target site contained within the one or more endogenous genes.

In some of any of the provided embodiments, the one or more nucleic acid molecules is comprised within a vector. In some of any of the provided embodiments, the vector is a donor vector. In some of any of the provided embodiments, the vector is an expression vector. In some of any of the provided embodiments, the vector is a viral vector. In some of any of the provided embodiments, the viral vector is selected from the group consisting of a retroviral vector, a gammaretroviral vector, a lentiviral vector, and an adeno-associated viral (AAV) vector. In some of any of the provided embodiments, the AAV vector is a self-complementary AAV (scAAV) vector. In some of any of the provided embodiments, the vector is a non-viral vector. In some of any of the provided embodiments, the vector is a donor vector for genome editing. In some of any of the provided embodiments, the vector is a transposon vector. In some of any of the provided embodiments, the transposon vector is a Sleeping Beauty transposon vector or a PiggyBac transposon vector. In some of any of the provided embodiments, the vector is suitable for gene editing or genomic engineering. In some of any of the provided embodiments, the introducing of one or more of: (i) the one or more agents capable of editing and/or disrupting one or more endogenous genes in the cell, (ii) one or more agents capable of inducing a DNA break in one or more endogenous genes in the cell, and/or (iii) one or more nucleic acid molecules, is carried out by transfection, electroporation, or transduction, or any combination thereof. In some of any of the provided embodiments, the introducing of one or more of: (i) the one or more agents capable of editing and/or disrupting one or more endogenous genes in the cell, (ii) one or more agents capable of inducing a DNA break in one or more endogenous genes in the cell, and/or (iii) one or more nucleic acid molecules, is carried out by transfection. In some of any of the provided embodiments, the introducing of the one or more nucleic acid molecules by transfection comprises introducing any vector provided herein into the cell. In some of any of the provided embodiments, the introducing one or more of: (i) the one or more agents capable of editing and/or disrupting one or more endogenous genes in the cell, (ii) one or more agents capable of inducing a DNA break in one or more endogenous genes in the cell, and/or (iii) one or more nucleic acid molecules, is carried out by electroporation. In some of any of the provided embodiments, the introducing one or more of: (i) the one or more agents capable of editing and/or disrupting one or more endogenous genes in the cell, (ii) one or more agents capable of inducing a DNA break in one or more endogenous genes in the cell, and/or (iii) one or more nucleic acid molecules, is carried out by transduction.

In some of any of the provided embodiments, the introducing the one or more nucleic acid molecules by transduction comprises introducing any vector provided herein into the cell. In some of any of the provided embodiments, the introducing the one or more nucleic acid molecules occurs by a transfection method. In some of any of the provided embodiments, the transfection method is electroporation.

In some of any of the provided embodiments, the cell is from a subject having a disease or condition associated with amyloid beta. In some of any of the provided embodiments, the disease or condition associated with amyloid beta is a disease or condition associated with human amyloid beta. In some of any of the provided embodiments, the disease or condition associated with amyloid beta is Alzheimer's Disease. In some of any of the provided embodiments, the cell is from a subject having Alzheimer's Disease. In some of any of the provided embodiments, the cell is from a donor subject. In some of any of the provided embodiments, the donor subject does not have a disease or condition associated with amyloid beta or has not been diagnosed as having a disease or condition associated with amyloid beta. In some of any of the provided embodiments, the donor subject does not have Alzheimer's Disease or has not been diagnosed as having Alzheimer's Disease. In some of any of the provided embodiments, the cell is a T cell, a B cell, or a Natural Killer (NK) cell. In some of any of the provided embodiments, the engineered cell is a T cell. In some of any of the provided embodiments, the T cell is a regulatory T cell or an effector T cell. In some of any of the provided embodiments, the T cell is a regulatory T cell. In some of any of the provided embodiments, the T cell is a CD4+ and/or CD8+ T cell. In some of any of the provided embodiments, the T cell is a CD4+/CD25+/FOXP3+ T cell.

In some aspects, provided herein is an engineered cell produced by any method provided herein. In some of any of the provided embodiments, the engineered cell is a regulatory T cell.

In some aspects, provided herein is a conjugate, comprising any TCR or antigen-binding fragment thereof provided herein, and a heterologous moiety. In some of any of the provided embodiments, the heterologous moiety is a detectable label. In some of any of the provided embodiments, the detectable label is a fluorescent label. In some of any of the provided embodiments, the detectable label is a radioisotope, a fluorescent label, or an enzyme-substrate.

In some aspects, provided herein is a composition, comprising any engineered cells, or any TCR or antigen-binding fragment thereof, or any conjugate provided herein. In some of any of the provided embodiments, the composition further comprises a pharmaceutically acceptable excipient.

In some aspects, provided herein is a method of treatment of a disease or condition associated with amyloid beta, comprising administering any engineered cell, or any composition provided herein, to a subject having a disease or condition associated with amyloid beta. In some of any of the provided embodiments, the disease or condition associated with amyloid beta is Alzheimer's Disease.

In some aspects, provided herein is a method of treatment of Alzheimer's Disease, comprising administering any engineered cell, or any composition provided herein, to a subject having Alzheimer's Disease.

In some aspects, provided herein is a method of treatment of a disease or condition associated with amyloid beta, comprising administering any composition provided herein to a subject having a disease or condition associated with amyloid beta. In some of any of the provided embodiments, the disease or condition associated with amyloid beta is Alzheimer's Disease.

In some aspects, provided herein is a method of treatment of a disease or condition associated with amyloid beta, comprising administering any composition provided herein to a subject having Alzheimer's Disease.

In some aspects, provided herein is a method of treating a disease or condition associated with amyloid beta, comprising: a) engineering a cell by any method provided herein; b) culturing the engineered cell under conditions to produce a population of engineered cells; and c) administering a therapeutically effective amount of the population of engineered cells to a subject having a disease or condition associated with amyloid beta.

In some aspects, provided herein is a method of treating Alzheimer's Disease, comprising: a) engineering a cell by any method provided herein; b) culturing the engineered cell under conditions to produce a population of engineered cells; and c) administering a therapeutically effective amount of the population of engineered cells to a subject having Alzheimer's Disease. In some of any of the provided embodiments, the subject is a human.

In some of any of the provided embodiments, the engineered cells are autologous to the subject. In some of any of the provided embodiments, the engineered cells are allogenic to the subject. In some of any of the provided embodiments, the engineered cells are T cells, B cells, or Natural Killer (NK) cells. In some of any of the provided embodiments, the engineered cells are T cells. In some of any of the provided embodiments, the T cells are regulatory T cells or effector T cells. In some of any of the provided embodiments, the T cells are regulatory T cells. In some of any of the provided embodiments, the T cells are CD4+ and/or CD8+ T cells. In some of any of the provided embodiments, the T cells are CD4+/CD25+/FOXP3+ T cells.

In some of any of the provided embodiments, a composition is for use in treating a disease or condition associated with amyloid beta in a subject.

In some of any of the provided embodiments, use of a composition is for the manufacture of a medicament for treating a disease or condition associated with amyloid beta in a subject.

In some of any of the provided embodiments, the subject is a human. In some of any of the provided embodiments, the disease or condition associated with amyloid beta is Alzheimer's Disease.

In some aspects, provided herein is a method of diagnosing a disease or condition associated with amyloid beta, comprising administering any TCR or antigen-binding fragment thereof, or any conjugate, or any composition provided herein, to a subject having or suspected of having a disease or condition associated with amyloid beta.

In some of any of the provided embodiments, the method further comprises detecting the level or absence of binding between the TCR or antigen-binding fragment thereof or the conjugate with amyloid beta. In some of any of the provided embodiments, the method further comprises comparing the level or absence of binding to the level or absence of binding between the TCR or antigen-binding fragment thereof or the conjugate with amyloid beta as detected as one or more preceding time points.

In some aspects, provided herein is a method of monitoring the progression of a disease or condition associated with amyloid beta, comprising administering any TCR or antigen-binding fragment thereof or any conjugate, or any composition provided herein, to a subject having a disease or condition associated with amyloid beta.

In some of any of the provided embodiments, the method further comprises detecting the level or absence of binding between the TCR or antigen-binding fragment thereof or the conjugate with amyloid beta. In some of any of the provided embodiments, the method further comprises comparing the level or absence of binding to the level or absence of binding between the TCR or antigen-binding fragment thereof or the conjugate with amyloid beta as detected as one or more preceding time points.

In some aspects, provided herein is a method of diagnosing Alzheimer's Disease, comprising administering any TCR or antigen-binding fragment thereof, or any conjugate, or any composition provided herein, to a subject having or suspected of having Alzheimer's Disease.

In some aspects, provided herein is a method of monitoring the progression of Alzheimer's Disease, comprising administering any TCR or antigen-binding fragment thereof, or any conjugate, or any composition provided herein, to a subject having Alzheimer's Disease.

In some of any of the provided embodiments, a composition is for use in treating a disease or condition associated with amyloid beta in a subject. In some of any of the provided embodiments, use of a composition is for the manufacture of a medicament for treating a disease or condition associated with amyloid beta in a subject.

In some of any of the provided embodiments, a composition is for use in treating Alzheimer's Disease in a subject. In some of any of the provided embodiments, use of a composition is for the manufacture of a medicament for treating Alzheimer's Disease in a subject. In some of any of the provided embodiments, the subject is a human.

### II. BRIEF DESCRIPTION OF THE DRAWINGS

FIG. 1 depicts the workflow showing development of antigen-specific T cells. Non-Tg mice immunized with Aβ₄₂ followed by isolation of CD4+ T cells from the spleen and lymph nodes. In vitro culture of CD4+ T cells in presence of feeder cells and Aβ₄₂ for enrichment of antigen-rich population. Growth pattern of CD4+ T cells over in vitro incubation where cells growth declined for several weeks followed by increase in cell number. Limiting dilution culture technique to seed as low as 1 cell per well in presence of feeder cells, Aβ₄₂, and Il2 to obtain monoclonal Aβ-Th1 cell clone. Aβ-Th1 cells polarized into the Aβ-Th17 cells using conditional culture media in presence of feeder cells and Aβ₄₂.

FIG. 2 shows flow cytometry analysis of the intracellular cytokine and transcription factor of the more than six months in vitro cultured Aβ-Th1 and Aβ-Th17 cells after stimulation with PMA and ionomycin in presence of brefeldin A.

FIG. 3 shows a representative immunoblot and quantification of 42 different cytokines and chemokines extracellularly secreted from the Aβ-Th1 and Aβ-Th17 cells after stimulation with PMA and ionomycin.

FIG. 4A shows MHCII-IA^{b}-KLVFFAEDVGSNKGA (Aβ T cell epitope) tetramer (MHC-peptide tetramer) binding with Aβ-Th1 and Aβ-Th17 cells after incubation at 37° C. for 3 hr. Control tetramer MHCII-IA^{b}-PVSKMRMATPLLMQA was used for precise gating of Aβ T cell epitope recognizing CD4+T cell population. FIG. 4B shows the three-month Aβ-Th1 and -Th17 clones binding with MHC-peptide tetramer in a dose-dependent manner. FIG. 4C shows prolonged maintenance of stable Teff clones potentiate cognate antigen recognition detected by improved dose-dependent Aβ-Teff-MHC-peptide binding.

FIG. 5A shows, from Aβ-Th1 cells, antigen recognizing variable regions of T cell receptor (TCR) alpha (α) and beta (β) chains identified using molecular cloning. Molecular modeling of full length TCRα/β complex with Aβ₄₂-MHCII (H-2b) (pMHCII) complex was performed to determine exact amino acid interactions between two complexes. pMHC complex: the MHCII molecule chain and the peptide chain are labeled accordingly. Two chains of TCR: the TCR-α chain and the TCR-β is are labeled accordingly. The complete model for the TCR-pMHC complex is also shown and enlarged focused region showing interaction between the pMHC with the TCR and the interacting residues. FIG. 5B shows (i) Peptide surface at the interface of MHC and TCR. The TCR-pMHC interactions-(ii) pMHC residues Gly111 (O) and Val112(O) interact with Lys479 and Asn481 sidechains of TCR-β, respectively, to form hydrogen bond interactions; (iii) The ring nitrogen (NE2) of pMHC His86 and His87 interacts with the TCR-α Glu184(OE1); (iv) Lys479 (NZ) forms weaker Van der Waals interactions with Met108(O, SD); (v) A π-π interaction is observed between peptide Phe93 and the Pro411 of TCR-chain due to stacking of aromatic rings.

FIG. 6A shows a schematic presentation of the in vivo experimental procedure performed in 4-5 months old female APP/PS1 and age matched non-Tg mice. n=6 mice per group were used. FIG. 6B depicts a radial arm water maze (RAWM) test performed in the experimental mice three weeks after the adoptive transfer of 2×10⁶ Aβ-Th1 and Aβ-Th17 cells intravenously. Cognitive errors of 9 days trials were divided into three blocks and averaged for the statistical analysis. Two-way ANOVA was used to determine significant differences between experimental groups.

FIG. 7A shows a 2-deoxy-glucose (2DG) CEST MRI performed five weeks after adoptive transfer of Aβ-Th1 and Aβ-Th17 cells. Fasting blood glucose measured followed by Gluco-CEST MRI scanning. FIG. 7B shows representative MRI images with hippocampal glucose signal for different experimental mice which include baseline MRI scan followed by 2DG injection and thereafter MRI scans every 10 min up to 1 h. n=4 mice per group were analyzed. Glucose intensity in the hippocampus was calculated as % ΔMTR at different time points compared to the baseline. Area under the curve (AUC) for each mouse was calculated and averaged for statistical significance using one-way ANOVA followed by Newman-Keuls post hoc test.

FIG. 8 shows the frequency of CD3+CD4+ and CD3+CD8+ T cells in experimental mice sacrificed six weeks after Aβ-Th1 and Aβ-Th17 cells adoptive transfer (n=6 mice per group were analyzed).

FIG. 9 shows the frequency of CD4+Tbet+ and CD4+RORγ+ T cells in experimental mice sacrificed six weeks after Aβ-Th1 and Aβ-Th17 cells adoptive transfer (n=6 mice per group were analyzed).

FIG. 10A shows the frequency of intracellular pro-inflammatory cytokines TNFα, IFNγ and IL17 from the splenocytes of different experimental mice after stimulation with PMA and ionomycin in presence of brefeldin A (n=6 mice per group were analyzed). FIG. 10B shows the frequency of Aβ reactive CD4+ T cells among lymph node cells after stimulation with Aβ₁₋₄₂ in presence of feeder cells using fluorescently labeled MHCII-IA^{b}-peptide tetramer (n=4 mice per group were analyzed).

FIG. 11A shows the frequency of CD4+CD25+ and CD4+FOXP3+ Treg in the blood, spleen and lymph nodes of different experimental mice (n=6 mice per group). FIG. 11B shows the immunosuppressive function of Treg assessed against proliferating Tresp isolated from non-Tg mice and assessed using a CFSE assay. Tregs isolated from different experimental groups (n=6 mice per group) were pooled and the assay was performed in triplicate. Statistical analysis was performed using linear regression analyses where non-Tg mice group showed R²=0.28 with P value=0.07 while all other APP/PS1 groups showed R²>0.65 with P values <0.01.

FIG. 12A shows transcriptomics analysis performed to determine the expression of different innate and adaptive immune genes using the RNA isolated from the hippocampal tissues of different experimental mice. First histogram: Fold changes in the expression of genes in different APP/PS1 mice groups compared to the non-Tg mice. Second histogram. Fold change in the APP/PS1/Aβ-Th1 and APP/PS1/Aβ-Th17 mice groups compared to the untreated APP/PS1 mice. Third histogram. p values APP/PS1/Aβ-Th1 and APP/PS1/Aβ-Th17 mice groups plotted compared to the untreated APP/PS1 mice. n=4 mice per group analyzed using Qiagen RT²-PCR array. FIG. 12B First histogram: gene expression in different APP/PS1 mice compared to the non-Tg mice. Second histogram: Gene expression in APP/PS1/Aβ-Th1 and APP/PS1/Aβ-Th17 mice compared to the untreated APP/PS1 mice. Fold changes in different innate and adaptive immune genes are presented as histograms for n=4 mice per group were analyzed using Qiagen RT²-PCr array.

FIG. 13 shows a gene network analysis performed using Ingenuity Pathway Analysis (IPA, Qiagen) and top canonical pathways affected in different APP/PS1 mice compared to the non-Tg mice.

FIG. 14 shows immunoprecipitation followed by western blot analyses using cortical tissue protein to determine expression of different Aβ oligomers. N=3 mice per group were analyzed.

FIG. 15A shows immunohistochemistry performed to determine the area occupied by the insoluble Aβ plaque in the cortex and hippocampal brain regions. Representative images showing 6E10 staining in different brain regions. Percentage occupied area quantified using Cavalieri estimator probe of Stereo Investigator system (MBF Bioscience). Scale bar=100 μm. N=6 mice per group were analyzed. FIG. 15B shows an ELISA to quantify Aβ₄₀ and Aβ₄₂ levels in the brain using Tris-HCl soluble fractions of cortical tissue. N=6 mice per group were analyzed. Statistical differences between experimental groups determined using one-way ANOVA followed by Newman-Keuls post-hoc test.

FIG. 16A shows immunohistochemistry performed to quantify number of reactive Iba1 positive microglia cells in the cortex and hippocampus brain regions. Representative pictures showing Iba1 positive cells and cell aggregates in different brain regions. Scale bar=100 μm. Area with most aggregated Iba1+ cells highlighted by inserts for the cortex and hippocampus. Scale bar=20 μm. FIG. 16B shows the number of Iba1+ cell aggregates were quantified using optical fractionator module of Stereo Investigator system (MBF Bioscience). N=5 mice per group were analyzed.

FIG. 17 shows a western blot analysis performed to determine expression of iNOS and arginase-1 using cortical tissue lysate for assessment of microglial phenotypes. Representative immunoblot and densitometric quantification were performed using n=4 mice per group. Statistical significance between different experimental groups determined using one-way ANOVA followed by Newman-Keuls post hoc test. *p<0.05, **p<0.01, ***p<0.001, ****p<0.0001.

FIG. 18 shows the neuronal progenitor cell density determined by immunohistochemical staining of doublecortin positive (Dcx+) cells in the dentate gyrus region of the hippocampus. Representative images showing Dcx+ cells are included. Scale bar=20 μm. Dcx+ cells were quantified using the optical fractionator module of Stereo Investigator system (MBF Bioscience). N=6 mice per group were analyzed.

FIG. 19 shows a western blot analysis performed to determine expression of presynaptic (synaptophysin) and postsynaptic (PSD95) proteins using cortical tissue protein. Representative immunoblot and densitometric quantification performed for n=4 mice per group. Statistical significance between different experimental groups determined using one-way ANOVA followed by Newman-Keuls post-hoc test.

FIG. 20 shows an example lentiviral vector for delivery of beta-amyloid specific TCR sequences.

### DETAILED DESCRIPTION

Alzheimer's Disease (AD) is the most common age-related dementia, and there remains a need for new therapies, including cell therapies, for the treatment of Alzheimer's Disease. AD is now the sixth leading cause of death in the United States. From 2000 to 2015, AD-associated mortalities have increased by 123%. More problematic is that AD cannot be prevented, cured, or slowed. This makes it one of the most important unmet medical need of our time as outlined in the Brain Research through Advancing Innovative Neurotechnologies (BRAIN) initiative of 2013. Existing small molecule therapies can provide only symptomatic relief and since the approval of last AD medicine in 2003, more than 400 drug candidates have failed in the clinical testing. The body's immune system plays both a protective and pathological role and such immune responses are disease specific. In AD, immune T cells become reactive against key pathological protein amyloid beta (may also be referred to as Aβ or A-beta or A beta). However, the frequency of pro-inflammatory and neurodestructive effector T cells (Teffs) reactive against Aβ increases over anti-inflammatory, immunosuppressive, and neuroprotective regulatory T cells (Tregs) leading to disease progression and worsening.

For either T cell subset, Aβ specificity is attributed to unique T cell receptor (TCR) variable regions. Therefore, high affinity monoclonal T cells that specifically recognize Aβ with very high binding affinity were generated as described herein; the unique TCR alpha (α) and beta (β) chain sequences were isolated and sequenced; and unique TCR was confirmed with molecular modeling, as shown in the Examples. The newly identified Aβ-T cells comprise a TCR that has utility in various ways, including in treatment, diagnosis, and monitoring of diseases or conditions associated with amyloid beta, and is also helpful for the study AD pathology, including in murine models. The unique Aβ-TCR sequence described herein can be used to engineer variant immune cells for the treatment of diseases or conditions associated with amyloid beta, such as AD. Moreover, such engineered cells and cell lines thereof can be prepared for use as a diagnostic measure for AD in the early stages of disease where other causes of dementia need be excluded for proper treatment and follow up.

Existing AD therapeutics provide only symptomatic relief and therefore development of better alternatives is very essential. Harnessing the body's immune cells for the treatment of AD is very unique approach and to date no successful curative or interdictory immunotherapy is available. Targeting pathological proteins is very challenging especially using immune cells which exhibit diverse phenotypes. The unique Aβ-TCR sequence disclosed herein, which recognizes a human pathological protein (human amyloid beta) can be used to engineer different immune T cells, e.g., Tregs, to program them to fight and regulate AD pathology. Moreover, the human Aβ recognizing capabilities will allow engineering of AD patients' T cells by replacing endogenous TCRs, e.g., the endogenous TRAC gene and/or TRBC gene, with a unique Aβ-TCR sequence, and thus personalize immune treatment for AD patients and patients having other diseases or conditions associated with amyloid beta.

Currently, polyclonal T cell therapies are under development for the treatment of autoimmune neurodegenerative diseases. However, such polyclonal T cells can be lethal for AD patients (NCT00021723). In contrast, development of monoclonal and disease specific T cell therapies can be effective for the management of AD. Also, there is no conclusive diagnostic test for AD and these cells acquired for people with, or at risk for, AD and their reactions of Aβ for cell proliferation can be used in this unique diagnostic niche.

Accordingly, as shown in the Examples, monoclonal T cell lines were generated that enabled specific recognition of the principal AD pathological protein, amyloid beta (Aβ). Human Aβ specific T cells are of the Th1 phenotype thought to play an important pathological role in disease and are now referred to as Aβ-Th1 cells. Indeed, T cell-mediated meningoencephalitis to Ab was the cause of serious adverse events leading to the termination of the Phase II trial for the AN1792 Ab vaccine (NCT00021723), thus underscoring the potential deleterious role of Ab-specific T cells to AD progression. The monoclonal Aβ-Th1 cells can be cultivated for more than six months and exhibit strong affinity to the human Aβ T cell epitope as confirmed by the Aβ-MHCII-IA^{b} tetramer staining. The antigen-specificity of the T cell is attributed to its unique T cell receptor (TCR) variable regions. Accordingly, TCR alpha (α) and beta (β) chain sequences of Aβ-Th1 cells were identified using molecular cloning and in silico modeling which further confirmed their Aβ specificity. The high-affinity Aβ-TCR can be used to engineer T cells, e.g., Treg cells, to combat disease pathobiology. Using this unique Aβ-TCR, the present disclosure involves, inter alia, engineering anti-inflammatory and immunosuppressive regulatory T cells (Aβ-Treg) for treatment of diseases and conditions associated with amyloid beta, such as AD, as well as the diagnosis and monitoring thereof. Thus, the present disclosure provides T cell receptors that recognize pathogenic peptides of Aβ, which can be used to engineer Aβ-specific cells, such as immune cells, e.g., Tregs (and other cell types), for the treatment, diagnosis, and/or monitoring of Alzheimer's Disease.

Accordingly, provided herein are T cell receptors (TCRs) or antigen-binding fragments thereof that bind to, e.g., specifically bind to, amyloid beta; conjugates comprising a TCR or antigen-binding fragment thereof; and engineered cells comprising a TCR or antigen-binding fragment thereof; along with nucleic acid molecules encoding the same and vectors comprising such nucleic acid molecules; compositions of any of such; and methods of production, methods of engineering and methods of treatment, diagnosis, and monitoring a disease or condition associated with or caused in whole or in part by amyloid beta, e.g., Alzheimer's Disease, that involves the use of any such TCR or antigen-binding fragment thereof, conjugate, or composition. The present disclosure also describes compositions for developing and manufacturing cell therapies for the treatment of Alzheimer's Disease and other diseases or pathologies that involve, are linked to, or are caused by (including caused in part by) amyloid-beta. Furthermore, this disclosure describes novel cell therapy products for the treatment, diagnosis, and monitoring of diseases and conditions associated with amyloid beta, e.g., Alzheimer's Disease and other diseases or pathologies that involve, are linked to, or are caused by (including caused in part by) amyloid-beta.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### III. T CELL RECEPTORS AND ANTIGEN-BINDING FRAGMENTS THEREOF

Provided herein are T cell receptor (TCRs) and antigen-binding fragments thereof, e.g., TCRs or antigen-binding fragments thereof that specifically bind to amyloid beta, e.g., human amyloid beta.

In some embodiments, the TCR or antigen-binding fragment thereof comprises an alpha chain comprising a variable alpha (Vα) region and a beta chain comprising a variable beta (Vβ) region, and the Vα region comprises a complementarity determining region 1 (CDR-1), CDR-2, and CDR-3 comprising the amino acid sequences of SEQ ID NOs: 12, 13, and 14, respectively; and the Vβ region comprises a CDR-1, CDR-2, and CDR-3 comprising the amino acid sequences of SEQ ID NOs: 15, 16, and 17, respectively.

In some embodiments, the TCR or antigen-binding fragment thereof comprises an alpha chain comprising a variable alpha (Vα) region and a beta chain comprising a variable beta (Vβ) region, and the Vα region comprises a complementarity determining region 1 (CDR-1), CDR-2, and CDR-3 comprising the CDR-1, CDR-2, and CDR-3 amino acid sequences contained within the amino acid sequence of SEQ ID NO: 2 or 5; and the Vβ region comprises a CDR-1, CDR-2, and CDR-3 comprising the CDR-1, CDR-2, and CDR-3 amino acid sequences contained within the amino acid sequence of SEQ ID NO: 4 or 8.

In some embodiments, the TCR or antigen-binding fragment thereof comprises an alpha chain comprising a variable alpha (Vα) region and a beta chain comprising a variable beta (Vβ) region, and the Vα region comprises a complementarity determining region 1 (CDR-1), CDR-2, and CDR-3 comprising the CDR-1, CDR-2, and CDR-3 amino acid sequences contained within the amino acid sequence of SEQ ID NO: 5; and the Vβ region comprises a CDR-1, CDR-2, and CDR-3 comprising the CDR-1, CDR-2, and CDR-3 amino acid sequences contained within the amino acid sequence of SEQ ID NO: 8. In some embodiments, the TCR or antigen-binding fragment thereof comprises an alpha chain comprising a variable alpha (Vα) region and a beta chain comprising a variable beta (Vβ) region, and the Vα region comprises a complementarity determining region 1 (CDR-1), CDR-2, and CDR-3 comprising the CDR-1, CDR-2, and CDR-3 amino acid sequences contained within the amino acid sequence of SEQ ID NO: 5; and the Vβ region comprises a CDR-1, CDR-2, and CDR-3 comprising the CDR-1, CDR-2, and CDR-3 amino acid sequences contained within the amino acid sequence of SEQ ID NO: 4.

It is within the level of a skilled artisan to determine or identify the various domains or regions of a TCR, including the variable regions, the constant regions, the CDR regions, among other domains. It is understood that reference to amino acid residues, including to a specific sequence as set forth by a SEQ ID NO as used to describe the organization of domains of a TCR are for illustrative purposes and are not meant to limit the scope of the embodiments provided herein. In some cases, the specific domain, e.g., a variable domain or a constant domain, can be several amino acids, e.g., one, two, three, or four, shorter or longer. In some aspects, the residues of a TCR are known or can be identified according to the International ImMunoGene Tics Information system (IMGT) numbering system. See, e.g., www.imgt.org; see also Lefranc et al., 2003, Developmental and Comparative Immunology, 27(1): 55-77; and The T Cell Factsbook, 2^{nd} Edition, Lefranc and Lefranc, Academic Press, 2001. Using the IMGT system, the CDR-1 sequences within a TCR Vα chain and/or a TCR Vβ chain correspond to the amino acid residues present between residues 27-38, inclusive, the CDR-2 sequences within a TCR Vα chain and/or a TCR Vβ chain correspond to the amino acid residues present between residues 56-65, inclusive, and the CDR-3 sequences within a TCR Vα chain and/or a TCR Vβ chain correspond to the amino acid residues present between residues 105-117, inclusive. Accordingly, in some embodiments, the CDR-1, CDR-2, and CDR-3 sequences of the TCR Vα chain and/or the TCR Vβ chain is determined or identified using IMGT numbering.

In some embodiments, the TCR or antigen-binding fragment thereof comprises a Vα region comprising the amino acid sequence of SEQ ID NO: 5 and a Vβ region comprising the amino acid sequence of SEQ ID NO: 8, or comprises a Vα region comprising an amino acid sequence having at least 90%, 91%, 92%, 93%, 94%, 95%, 96%, 97%, 98%, or 99% sequence identity to SEQ ID NO: 5, and comprises a Vβ region comprising an amino acid sequence having at least 90%, 91%, 92%, 93%, 94%, 95%, 96%, 97%, 98%, or 99% sequence identity to SEQ ID NO: 8. In some embodiments, the TCR or antigen-binding fragment thereof comprises a Vα region comprising an amino acid sequence having at least 99% sequence identity to SEQ ID NO: 5, and comprises a Vβ region comprising an amino acid sequence having at least 99% sequence identity to SEQ ID NO: 8. In some embodiments, the TCR or antigen-binding fragment thereof comprises a Vα region comprising the amino acid sequence of SEQ ID NO: 5 and a Vβ region comprising the amino acid sequence of SEQ ID NO: 8.

In some embodiments, the TCR or antigen-binding fragment thereof comprises an alpha chain comprising the amino acid sequence of SEQ ID NO: 2 or an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 2; and/or the TCR or antigen-binding fragment thereof comprises a beta chain comprising the amino acid sequence of SEQ ID NO: 4 or an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 4. In some embodiments, the TCR or antigen-binding fragment thereof comprises an alpha chain comprising the amino acid sequence of SEQ ID NO: 2 or an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 2; and the TCR or antigen-binding fragment thereof comprises a beta chain comprising the amino acid sequence of SEQ ID NO: 4 or an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 4. In some embodiments, the TCR or antigen-binding fragment thereof comprises an alpha chain comprising the amino acid sequence of SEQ ID NO: 2; and/or the TCR or antigen-binding fragment thereof comprises a beta chain comprising the amino acid sequence of SEQ ID NO: 4.

In some embodiments, the TCR or antigen-binding fragment thereof comprises an alpha chain comprising a Vα region linked to a junction region and the junction region is further linked to a Cα region.

In some embodiments, the TCR or antigen-binding fragment thereof comprises a beta chain comprising a Vβ region linked to a diversity region and the diversity region is further linked to a junction region and the junction region is further linked to a Cβ region.

In some embodiments, the TCR or antigen-binding fragment thereof comprises an alpha chain comprising a Vα region linked to a junction region and the junction region is further linked to a Cα region; and the TCR or antigen-binding fragment thereof comprises a beta chain comprising a Vβ region linked to a diversity region and the diversity region is further linked to a junction region and the junction region is further linked to a Cβ region.

In some of any of such embodiments, the TCR or antigen-binding fragment thereof specifically binds to amyloid beta. In some embodiments, the TCR or antigen-binding fragment thereof specifically binds to human amyloid beta.

In some embodiments, the alpha chain of the TCR or antigen-binding fragment thereof comprises a junction region. In some embodiments, the alpha chain comprises a junction region comprising the amino acid sequence of SEQ ID NO: 6, or an amino acid sequence having at least 80%, 85%, 90%, or 95% sequence identity to the amino acid sequence of SEQ ID NO: 6.

In some embodiments, the beta chain of the TCR or antigen-binding fragment thereof comprises a junction region. In some embodiments, the beta chain comprises a junction region comprising the amino acid sequence of SEQ ID NO: 10, or an amino acid sequence having at least 80%, 85%, 90%, or 95% sequence identity to the amino acid sequence of SEQ ID NO: 10.

In some embodiments, the beta chain of the TCR or antigen-binding fragment thereof comprises a diversity region. In some embodiments, the beta chain comprises a diversity region comprising the amino acid sequence of SEQ ID NO: 9 or an amino acid sequence having at least 40% of 70% sequence identity to the amino acid sequence of SEQ ID NO: 9.

In some embodiments, the alpha chain further comprises an alpha constant (Cα) region and/or the beta chain further comprises a beta constant (Cβ) region. In some embodiments, the alpha chain further comprises an alpha constant (Cα) region and the beta chain further comprises a beta constant (Cβ) region.

In some embodiments, the TCR or antigen-binding fragment thereof is murine, chimeric, or humanized.

In some embodiments, the TCR or antigen-binding fragment thereof is chimeric or humanized. A TCR or antigen-binding fragment thereof that is chimeric or humanized would be expected to exhibit improved host adherence and/or less immunogenicity in a human. In some embodiments, the TCR or antigen-binding fragment thereof is chimeric. In some embodiments, the TCR or antigen-binding fragment thereof is humanized. In some embodiments, the Cα region and/or the Cβ region is derived from a human TCR. In some embodiments, the Cα region and the Cβ region are derived from a human TCR. In some embodiments, the Cα region and/or the Cβ region are humanized, e.g., by introducing amino acid substitutions that reduce immunogenicity in a human. In some embodiments, the TCR or antigen-binding fragment thereof is chimeric and comprises the Vα and Vβ regions having an amino acid sequence as set forth in SEQ ID NOs: 5 and 8, respectively, or an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 5 and 8, respectively; and is linked to a Cα region and Cβ region derived from a human TCR.

In some embodiments, the Cα region comprises the amino acid sequence of SEQ ID NO: 7, or an amino acid sequence having at least 80%, 85%, 86%, 87%, 88%, 89%, 90%, 91%, 92%, 93%, 94%, 95%, 96%, 97%, 98%, or 99% sequence identity to the amino acid sequence of SEQ ID NO: 7.

In some embodiments, the Cβ region comprises the amino acid sequence of SEQ ID NO: 11, or an amino acid sequence having at least 80%, 85%, 86%, 87%, 88%, 89%, 90%, 91%, 92%, 93%, 94%, 95%, 96%, 97%, 98%, or 99% sequence identity to the amino acid sequence of SEQ ID NO: 11.

In some embodiments, the Cα region comprises the amino acid sequence of SEQ ID NO: 7, or an amino acid sequence having at least 80%, 85%, 86%, 87%, 88%, 89%, 90%, 91%, 92%, 93%, 94%, 95%, 96%, 97%, 98%, or 99% sequence identity to the amino acid sequence of SEQ ID NO: 7; and the Cβ region comprises the amino acid sequence of SEQ ID NO: 11, or an amino acid sequence having at least 80%, 85%, 86%, 87%, 88%, 89%, 90%, 91%, 92%, 93%, 94%, 95%, 96%, 97%, 98%, or 99% sequence identity to the amino acid sequence of SEQ ID NO: 11.

Also provided herein is a conjugate comprising any TCR or antigen-binding fragment thereof described herein, and a heterologous moiety. The heterologous moiety is not limited and can include a moiety useful for detection and/or treatment of a disease or condition involving and/or associated with amyloid beta, such as Alzheimer's Disease. In some embodiments, the heterologous moiety is a detectable label. The detectable label can be any label suitable for detection in vitro, in vivo, or ex vivo. In some embodiments, the detectable label is a fluorescent label. In some embodiments, the detectable label is a radioisotope, a fluorescent label, or an enzyme-substrate.

Among the provided embodiments is a TCR or antigen-binding fragment thereof comprising the CDR sequences and/or the variable regions and/or other sequences of the Aβ-Th1 TCR described in the Examples.

### IV. ENGINEERED CELLS

Provided herein are engineered cells, e.g., engineered T cells, such as regulatory T (Treg) cells that comprise a heterologous TCR or antigen-binding fragment thereof, such as any TCR or antigen-binding fragment thereof as described in Section III. Also provided herein are engineered cells, e.g., engineered T cells, such as regulatory T (Treg) cells that comprise a heterologous TCR or antigen-binding fragment thereof, such as any TCR or antigen-binding fragment thereof as described in Section III, and a genetic disruption in a T Cell Receptor Alpha Constant (TRAC) gene and/or a T Cell Receptor Beta Constant (TRBC) gene.

Provided herein, in some embodiments, is an engineered cell, comprising a heterologous T cell receptor (TCR) or antigen-binding fragment thereof, wherein the heterologous TCR or antigen-binding fragment thereof comprises an alpha chain comprising a variable alpha (Vα) region and a beta chain comprising a variable beta (Vβ) region, and wherein the Vα region comprises a complementarity determining region 1 (CDR-1), CDR-2, and CDR-3 comprising the amino acid sequences of SEQ ID NOs: 12, 13, and 14, respectively; and the Vβ region comprises a CDR-1, CDR-2, and CDR-3 comprising the amino acid sequences of SEQ ID NOs: 15, 16, and 17, respectively.

Provided herein, in some embodiments, is an engineered cell, comprising a heterologous T cell receptor (TCR) or antigen-binding fragment thereof, wherein the heterologous TCR or antigen-binding fragment thereof comprises an alpha chain comprising a variable alpha (Vα) region and a beta chain comprising a variable beta (Vβ) region, and wherein the Vα region comprises a complementarity determining region 1 (CDR-1), CDR-2, and CDR-3 comprising the CDR-1, CDR-2, and CDR-3 amino acid sequences contained within the amino acid sequence of SEQ ID NO: 2 or 5; and the Vβ region comprises a CDR-1, CDR-2, and CDR-3 comprising the CDR-1, CDR-2, and CDR-3 amino acid sequences contained within the amino acid sequence of SEQ ID NO: 4 or 8.

Provided herein, in some embodiments, is an engineered regulatory T (Treg) cell, comprising a heterologous T cell receptor (TCR) or antigen-binding fragment thereof, wherein the heterologous TCR or antigen-binding fragment thereof comprises an alpha chain comprising a variable alpha (Vα) region and a beta chain comprising a variable beta (Vβ) region, and wherein the Vα region comprises a complementarity determining region 1 (CDR-1), CDR-2, and CDR-3 comprising the amino acid sequences of SEQ ID NOs: 12, 13, and 14, respectively; and the Vβ region comprises a CDR-1, CDR-2, and CDR-3 comprising the amino acid sequences of SEQ ID NOs: 15, 16, and 17, respectively.

Provided herein, in some embodiments, is an engineered regulatory T (Treg) cell, comprising a heterologous T cell receptor (TCR) or antigen-binding fragment thereof, wherein the heterologous TCR or antigen-binding fragment thereof comprises an alpha chain comprising a variable alpha (Vα) region and a beta chain comprising a variable beta (Vβ) region, and wherein the Vα region comprises a complementarity determining region 1 (CDR-1), CDR-2, and CDR-3 comprising the CDR-1, CDR-2, and CDR-3 amino acid sequences contained within the amino acid sequence of SEQ ID NO: 2 or 5; and the Vβ region comprises a CDR-1, CDR-2, and CDR-3 comprising the CDR-1, CDR-2, and CDR-3 amino acid sequences contained within the amino acid sequence of SEQ ID NO: 4 or 8.

In some embodiments, the engineered cell further comprises a genetic disruption in one or more genes endogenous to the cell. In some embodiments, the one or more genes endogenous to the cell comprises a T Cell Receptor Alpha Constant (TRAC) gene and/or a T Cell Receptor Beta Constant (TRBC) gene.

Also provided herein is a engineered cell, comprising: a heterologous T cell receptor (TCR) or antigen-binding fragment thereof, wherein the heterologous TCR or antigen-binding fragment thereof comprises an alpha chain comprising a variable alpha (Vα) region and a beta chain comprising a variable beta (Vβ) region, and wherein the Vα region comprises a complementarity determining region 1 (CDR-1), CDR-2, and CDR-3 comprising the amino acid sequences of SEQ ID NOs: 12, 13, and 14, respectively; and the Vβ region comprises a CDR-1, CDR-2, and CDR-3 comprising the amino acid sequences of SEQ ID NOs: 15, 16, and 17, respectively; and a genetic disruption in a T Cell Receptor Alpha Constant (TRAC) gene and/or a T Cell Receptor Beta Constant (TRBC) gene.

Also provided herein is an engineered cell, comprising: a heterologous T cell receptor (TCR) or antigen-binding fragment thereof, wherein the heterologous TCR or antigen-binding fragment thereof comprises an alpha chain comprising a variable alpha (Vα) region and a beta chain comprising a variable beta (Vβ) region, and wherein the Vα region comprises a complementarity determining region 1 (CDR-1), CDR-2, and CDR-3 comprising the CDR-1, CDR-2, and CDR-3 amino acid sequences contained within the amino acid sequence of SEQ ID NO: 2 or 5; and the Vβ region comprises a CDR-1, CDR-2, and CDR-3 comprising the CDR-1, CDR-2, and CDR-3 amino acid sequences contained within the amino acid sequence of SEQ ID NO: 4 or 8; and a genetic disruption in a T Cell Receptor Alpha Constant (TRAC) gene and/or a T Cell Receptor Beta Constant (TRBC) gene.

Also provided herein is a engineered regulatory T (Treg) cell, comprising: a heterologous T cell receptor (TCR) or antigen-binding fragment thereof, wherein the heterologous TCR or antigen-binding fragment thereof comprises an alpha chain comprising a variable alpha (Vα) region and a beta chain comprising a variable beta (Vβ) region, and wherein the Vα region comprises a complementarity determining region 1 (CDR-1), CDR-2, and CDR-3 comprising the amino acid sequences of SEQ ID NOs: 12, 13, and 14, respectively; and the Vβ region comprises a CDR-1, CDR-2, and CDR-3 comprising the amino acid sequences of SEQ ID NOs: 15, 16, and 17, respectively; and a genetic disruption in a T Cell Receptor Alpha Constant (TRAC) gene and/or a T Cell Receptor Beta Constant (TRBC) gene.

Also provided herein is an engineered regulatory T (Treg) cell, comprising: a heterologous T cell receptor (TCR) or antigen-binding fragment thereof, wherein the heterologous TCR or antigen-binding fragment thereof comprises an alpha chain comprising a variable alpha (Vα) region and a beta chain comprising a variable beta (Vβ) region, and wherein the Vα region comprises a complementarity determining region 1 (CDR-1), CDR-2, and CDR-3 comprising the CDR-1, CDR-2, and CDR-3 amino acid sequences contained within the amino acid sequence of SEQ ID NO: 2 or 5; and the Vβ region comprises a CDR-1, CDR-2, and CDR-3 comprising the CDR-1, CDR-2, and CDR-3 amino acid sequences contained within the amino acid sequence of SEQ ID NO: 4 or 8; and a genetic disruption in a T Cell Receptor Alpha Constant (TRAC) gene and/or a T Cell Receptor Beta Constant (TRBC) gene.

In some embodiments, the engineered cell further comprises a nucleic acid molecule encoding the TCR or antigen-binding fragment thereof, such as any TCR or antigen-binding fragment thereof described herein, e.g., in Section III. The engineered cell can, in some embodiments, be engineered in various ways such that the nucleic acid molecule is capable of encoding the TCR or antigen-binding fragment thereof. In some embodiments, the nucleic acid molecule is under the control of one or more endogenous gene promoters. In some embodiments, the one or more endogenous gene promoters comprises a gene promoter associated with a TCR gene. In some embodiments, the one or more endogenous gene promoters comprises the endogenous TRAC gene promoter and/or the endogenous TRBC gene promoter. In some embodiments, the one or more endogenous gene promoters comprises the endogenous TRAC gene promoter. In some embodiments, the one or more endogenous gene promoters comprises the endogenous TRBC gene promoter. In some embodiments, the one or more endogenous gene promoters comprises the endogenous TRAC gene promoter and the endogenous TRBC gene promoter. In some embodiments, the nucleic acid molecule is under the control of an exogenous promoter.

In some embodiments, the engineered cell comprises an endogenous TRAC gene and/or an endogenous TRBC gene that has been disrupted and comprises a nucleic acid molecule encoding the TCR or antigen-binding fragment thereof that has been inserted into the disrupted endogenous TRAC gene and/or an endogenous TRBC gene. In some embodiments, the disrupted TRAC gene and/or an endogenous TRBC gene results in an absence of expression of, or reduced expression of, or lack of functional activity by, the gene product, e.g., TCR alpha and/or beta chain, encoded by the disrupted gene. By reducing, eliminating, knocking out, or disrupting the genes and/or gene products of the one or more genes endogenous to the cell, e.g., TRAC gene and/or TRBC gene, this can reduce or prevent expression of the endogenous TCR in the cell, e.g., the T cell, such as Treg cell, and/or an alpha or beta chain thereof. In some embodiments, reducing or preventing expression of the endogenous TCR in the cell, e.g., the T cell, such as Treg cell, and/or an alpha or beta chain thereof, can result in a lesser or reduced risk or chance or likelihood that the alpha and/or beta chains of the heterologous TCR or antigen-binding fragment thereof mispair with the endogenous TCR or alpha or beta chains thereof. Such mispairing could result in a TCR that differs from both the heterologous TCR or antigen-binding fragment thereof described herein and the endogenous TCR, which could potentially result in negative effects, such as different antigen recognition and/or specificity and/or altered, e.g., lower, expression levels of the endogenous TCR.

In some embodiments, the engineered cell is engineered to stably express a nucleic acid molecule encoding the TCR or antigen-binding fragment thereof, such as any TCR or antigen-binding fragment thereof described herein, e.g., in Section III. In some embodiments, the engineered cell is engineered to transiently express a nucleic acid molecule encoding the TCR or antigen-binding fragment thereof, such as any TCR or antigen-binding fragment thereof described herein, e.g., in Section III.

In some embodiments, the engineered cell further comprises a detectable moiety. The detectable moiety can be any detectable moiety suitable for detection using any in vivo, in vitro, or ex vivo detection methods.

Also provided herein is an engineered cell, comprising: a nucleic molecule encoding a heterologous T cell receptor (TCR) or antigen-binding fragment thereof, wherein the heterologous TCR or antigen-binding fragment thereof comprises an alpha chain comprising a variable alpha (Vα) region and a beta chain comprising a variable beta (Vβ) region, and wherein the Vα region comprises a complementarity determining region 1 (CDR-1), CDR-2, and CDR-3 comprising the amino acid sequences of SEQ ID NOs: 12, 13, and 14, respectively; and the Vβ region comprises a CDR-1, CDR-2, and CDR-3 comprising the amino acid sequences of SEQ ID NOs: 15, 16, and 17, respectively; and a genetic disruption in a T Cell Receptor Alpha Constant (TRAC) gene and/or a T Cell Receptor Beta Constant (TRBC) gene.

Also provided herein is an engineered cell, comprising: a nucleic acid molecule encoding a heterologous T cell receptor (TCR) or antigen-binding fragment thereof, wherein the heterologous TCR or antigen-binding fragment thereof comprises an alpha chain comprising a variable alpha (Vα) region and a beta chain comprising a variable beta (Vβ) region, and wherein the Vα region comprises a complementarity determining region 1 (CDR-1), CDR-2, and CDR-3 comprising the CDR-1, CDR-2, and CDR-3 amino acid sequences contained within the amino acid sequence of SEQ ID NO: 2 or 5; and the Vβ region comprises a CDR-1, CDR-2, and CDR-3 comprising the CDR-1, CDR-2, and CDR-3 amino acid sequences contained within the amino acid sequence of SEQ ID NO: 4 or 8; and a genetic disruption in a T Cell Receptor Alpha Constant (TRAC) gene and/or a T Cell Receptor Beta Constant (TRBC) gene.

The engineered cell can, in some embodiments, be engineered in various ways such that the nucleic acid molecule is capable of encoding the TCR or antigen-binding fragment thereof. In some embodiments, the nucleic acid molecule is under the control of one or more endogenous gene promoters. In some embodiments, the one or more endogenous gene promoters comprises a gene promoter associated with a TCR gene. In some embodiments, the one or more endogenous gene promoters comprises the endogenous TRAC gene promoter and/or the endogenous TRBC gene promoter. In some embodiments, the one or more endogenous gene promoters comprises the endogenous TRAC gene promoter. In some embodiments, the one or more endogenous gene promoters comprises the endogenous TRBC gene promoter. In some embodiments, the one or more endogenous gene promoters comprises the endogenous TRAC gene promoter and the endogenous TRBC gene promoter. In some embodiments, the nucleic acid molecule is under the control of an exogenous promoter.

In some embodiments, the engineered cell is engineered to stably express the nucleic acid molecule encoding the TCR or antigen-binding fragment thereof, such as any TCR or antigen-binding fragment thereof described herein, e.g., in Section III. In some embodiments, the engineered cell is engineered to transiently express the nucleic acid molecule encoding the TCR or antigen-binding fragment thereof, such as any TCR or antigen-binding fragment thereof described herein, e.g., in Section III. In some embodiments, the nucleic acid molecule is under the control of the endogenous TRAC gene promoter and/or the endogenous TRBC gene promoter. In some embodiments, the nucleic acid molecule is under the control of an exogenous promoter.

In some embodiments, the engineered cell is an immune cell. In some embodiments, the immune cell is a T cell, a B cell, or a Natural Killer (NK) cell. In some embodiments, the engineered cell is a T cell, a B cell, or a Natural Killer (NK) cell. In some embodiments, the engineered cell is a T cell. In some embodiments, the T cell is a regulatory T (Treg) cell or an effector T cell. In some embodiments, the T cell is a regulatory T (Treg) cell.

In some embodiments, the cell is a human cell, e.g., a human T cell, such as a human Treg cell. In some embodiments, the cell is obtained from a subject, e.g., a subject having a disease or condition associated with amyloid beta, e.g., Alzheimer's Disease; or a donor, e.g., a donor who does not have or is not suspected of having a disease or condition associated with amyloid beta, e.g., Alzheimer's Disease. In some embodiments, the cell is from a subject having a disease or condition associated with amyloid beta, e.g., Alzheimer's Disease. In some embodiments, the cell is from a donor subject. In some embodiments, the donor subject does not have a disease or condition associated with amyloid beta or has not been diagnosed as having a disease or condition associated with amyloid beta. In some embodiments, the donor subject does not have Alzheimer's Disease or has not been diagnosed as having Alzheimer's Disease, or does not have a disease or condition associated with amyloid beta or has not been diagnosed as having a disease or condition associated with amyloid beta. In some embodiments, the cell is an isolated cell, e.g., an isolated cell from a subject having Alzheimer's Disease, or an isolated cell from a donor subject. Accordingly, in some embodiments, the cell is an isolated cell that is obtained directly from a subject having a disease or condition associated with amyloid beta, e.g., Alzheimer's Disease. In some embodiments, the cell is an isolated cell is an allograft or a cell that is obtained directly from a donor subject, e.g., a subject other than the subject to which the engineered cells are to be administered, such as for treatment of a disease or condition associated with amyloid beta, such as Alzheimer's Disease. In some embodiments, the cell is derived from a subject by leukapheresis.

In some embodiments, the engineered cell comprises a vector, e.g., expression vector, such as a viral vector-based expression system, for expressing the TCR or antigen-binding fragment thereof.

In some embodiments, the TCR or antigen-binding fragment thereof has been introduced into the engineered cell by a genome editing technique, such that the endogenous TRAC gene and/or the endogenous TRBC gene has been edited and/or disrupted and a nucleic acid molecule encoding the TCR or antigen-binding fragment thereof has been inserted into the endogenous TRAC gene and/or the endogenous TRBC gene that has been edited and/or disrupted, e.g., by use of CRISPR-Cas9.

Also provided herein is an engineered cell comprising any of the nucleic acid molecules described herein, e.g., any of the nucleic acid molecules encoding any of the TCR or antigen-binding fragment thereof as described herein, e.g., as described in Section V.

Also provided herein is an engineered cell comprising any of the TCRs or antigen-binding fragments therein described herein, e.g., as described in Section IV.

In some of any of such embodiments, the TCR or antigen-binding fragment thereof is heterologous to the engineered cell.

Also provided herein is an engineered cell produced by any method described herein, e.g., any of the methods described in Section VI.

### V. NUCLEIC ACIDS AND VECTORS

Also provided herein is a nucleic acid molecule encoding any of the TCR or antigen-binding fragment thereof as described herein, e.g., any TCR or antigen-binding fragment therein as described in Section III, or an alpha or beta chain thereof, or any portion or component thereof. In some embodiments, the nucleic acid molecule is an isolated nucleic acid molecule. Accordingly, in some embodiments, provided herein is an isolated nucleic acid molecule encoding any of the TCR or antigen-binding fragment thereof as described herein, e.g., any TCR or antigen-binding fragment therein as described in Section III, or an alpha or beta chain thereof, or any portion or component thereof.

In some embodiments, the nucleic acid molecule is isolated, e.g., an isolated nucleic acid molecule.

The nucleic acid molecule can optionally be codon-optimized. In some embodiments, the nucleic acid molecule is codon-optimized. In some embodiments, the nucleic acid molecule is not codon-optimized.

In some embodiments, the nucleic acid molecule is DNA, e.g., cDNA. In some embodiments, the DNA is cDNA. In some embodiments, the nucleic acid molecule is RNA.

In some embodiments, the TCR or antigen-binding fragment thereof that is encoded by the nucleic acid molecule is humanized or chimeric. In some embodiments, the TCR or antigen-binding fragment thereof that is encoded by the nucleic acid molecule is murine.

In some embodiments, the nucleic acid molecule comprises the nucleic acid sequence of SEQ ID NO: 1, or a nucleic acid sequence having at least 80%, 85%, 90%, 91%, 92%, 93%, 94%, 95%, 96%, 97%, 98%, or 99% sequence identity to the nucleic acid sequence of SEQ ID NO: 1; and/or the nucleic acid sequence of SEQ ID NO: 3, or a nucleic acid sequence having at least 80%, 85%, 90%, 91%, 92%, 93%, 94%, 95%, 96%, 97%, 98%, or 99% sequence identity to the nucleic acid sequence of SEQ ID NO: 3.

In some embodiments, the nucleic acid molecule comprises a nucleic acid sequence encoding a Vα region comprising a CDR-1, CDR-2, and CDR-3 comprising the amino acid sequences of SEQ ID NOs: 12, 13, and 14, respectively; and/or comprises a nucleic acid sequence encoding a Vβ region comprising a CDR-1, CDR-2, and CDR-3 comprising the amino acid sequences of SEQ ID NOs: 15, 16, and 17, respectively.

In some embodiments, the nucleic acid molecule comprises a nucleic acid sequence encoding a Vα region comprising the amino acid sequence of SEQ ID NO: 5 or an amino acid sequence having at least 90%, 91%, 92%, 93%, 94%, 95%, 96%, 97%, 98%, or 99% sequence identity to the amino acid sequence of SEQ ID NO: 5; and/or comprises a nucleic acid sequence encoding a Vβ region comprising the amino acid sequence of SEQ ID NO: 8 or an amino acid sequence having at least 90%, 91%, 92%, 93%, 94%, 95%, 96%, 97%, 98%, or 99% sequence identity to the amino acid sequence of SEQ ID NO: 8.

In some embodiments, the nucleic acid molecule, e.g., DNA or RNA, encoding any TCR or antigen-binding fragment therein described herein, or a portion thereof, is contained or comprised within a vector or a plasmid, such as an expression vector or expression plasmid, including any vector or plasmid described herein.

Also provided herein is a vector comprising any of the nucleic acid molecules provided herein, e.g., any of the nucleic acid molecules encoding any of the TCR or antigen-binding fragments thereof as described herein, e.g., any TCR or antigen-binding fragment therein as described in Section III, or an alpha or beta chain thereof.

The vector can, in some embodiments, be any vector suitable for allowing for expression of the TCR or antigen-binding fragment therein in the cell, e.g., in the engineered cell. In some embodiments, the vector is an expression vector. In some embodiments, the vector is a plasmid, e.g., an expression plasmid.

In some embodiments, the vector is a viral vector. The viral vector can be any suitable viral vector and is not limited. In some embodiments, the viral vector is selected from the group consisting of a retroviral vector, a gammaretroviral vector, a lentiviral vector, and an adeno-associated viral (AAV) vector. In some embodiments, the AAV vector is a self-complementary AAV (scAAV) vector.

In some embodiments, the vector is an AAV vector. The AAV vector is not limited and may be any AAV vector. In some embodiments, the AAV vector is selected from the group consisting of AAV1, AAV2, AAV4, AAβ5, AAV6, AAV8, AAV9, and AAV-DJ/8 vectors.

In some embodiments, the vector is a retroviral vector. The retroviral vector is not limited and may be any retroviral vector. In some embodiments, the retroviral vector is a murine leukemia virus (MLV) vector or a feline leukemia virus (FeLV) vector. In some embodiments, the retroviral vector has a long terminal repeat sequence (LTR), such as a retroviral vector derived from a Moloney murine leukemia virus (MoMLV), a myeloproliferative sarcoma virus (MPSV), a murine stem cell virus (MSCV), a murine embryonic stem cell virus (MESV), or a spleen focus forming virus (SFFV). Accordingly, in some embodiments, the retroviral vector is selected from the group consisting of MLV, FeLV, MoMLV, MPSV, MSCV, MESV, and SFFV. Exemplary retroviral systems have been described, e.g., in U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

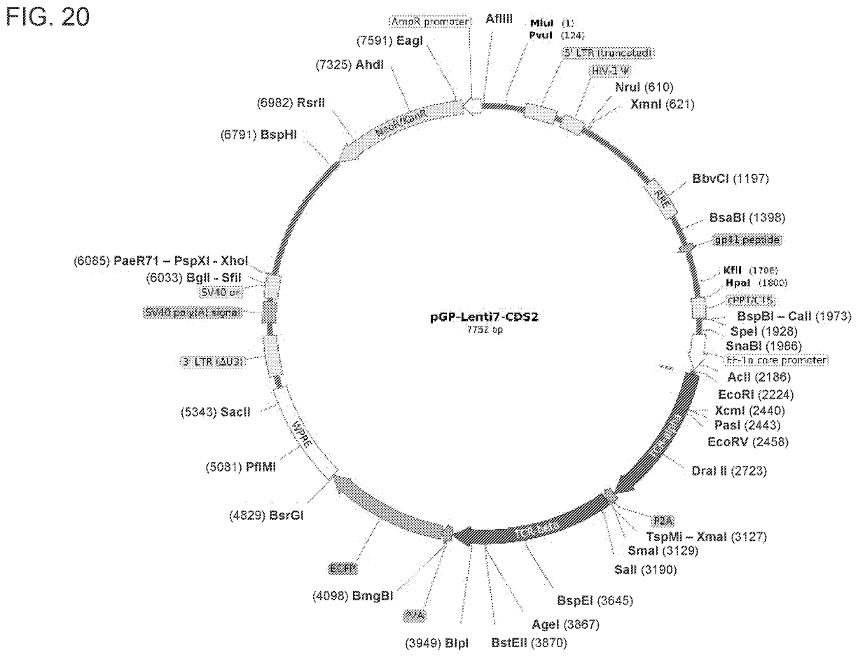

In some embodiments, the vector is a lentiviral vector. The lentiviral vector is not limited and may be any lentiviral vector. In some embodiments, the lentiviral vector is a pGP-Lenti7 lentiviral vector, e.g., pGP-Lenti7-CD52 lentiviral vector, such as depicted in FIG. 20. In some embodiments, the lentiviral vector is a VSV-G pseudotyped lentiviral vector. In some embodiments, the lentiviral vector is a human immunodeficiency virus (HIV)-derived lentiviral vector or a simian immunodeficiency virus (SIV)-derived lentiviral vector. In some embodiments, the HIV-derived lentiviral vector is derived from HIV-1 or HIV-2. Exemplary lentiviral systems and methods for lentiviral transduction are known, including, e.g., those described in, e.g., Cavalieri et al. (2003) Blood. 102(2): 497-505; Cooper et al. (2003) Blood. 101:1637-1644; Verhoeyen et al. (2009) Methods Mol Biol. 506:97-114; and Wang et al. (2012) J. Immunother. 35(9): 689-701.

In some embodiments, the vector is a donor vector, e.g., a donor plasmid, for genome editing. The donor vector can be any donor vector or plasmid suitable for use in genome editing techniques.

In some embodiments, the vector is a transposon vector. The transposon vector can be any transposon vector suitable for genetic engineering of cells. In some embodiments, the transposon vector is a Sleeping Beauty transposon vector or a PiggyBac transposon vector. In some embodiments, the transposon vector is a Sleeping Beauty transposon vector. In some embodiments, the transposon vector is a PiggyBac transposon vector.

In some embodiments, the vector is a non-viral vector. The non-viral vector can be any non-viral vector suitable for introducing the nucleic acid molecule into the cell.

### VI. METHODS OF PRODUCING CELLS AND ENGINEERING CELLS

Also provided herein are methods of producing engineered cells, methods of engineering cells, and methods of modifying cells, and related methods. In some embodiments, the methods comprise introducing any of the nucleic acid molecules or vectors described herein into a cell, e.g., an immune cell, such as a T cell, e.g., regulatory T cell.

Specifically, provided herein is a method of producing an engineered cell, comprising introducing any of the nucleic acid molecules described herein, e.g., any nucleic acid molecule encoding any TCR or antigen-binding fragment thereof, or a Vα region or Vβ region thereof, or any vector described herein, into a cell; and, optionally incubating the cell under conditions to allow for expansion of the cell into a population of engineered cells.

Also provided herein is a method of producing any of the engineered cells described herein, e.g., any of the engineered cells as described herein, such as in Section IV, comprising introducing any of the nucleic acid molecules described herein, e.g., any nucleic acid molecule encoding any TCR or antigen-binding fragment thereof, or a Vα region or Vβ region thereof, or any of the vectors described herein, into a cell; and, optionally incubating the cell under conditions to allow for expansion of the cell into a population of engineered cells.

In some embodiments, the method is performed in vitro or ex vivo. In some embodiments, the method is performed in vitro. In some embodiments, the method is performed ex vivo.

In some embodiments, the method involves engineering cells that comprise a TCR or antigen-binding fragment thereof that is murine, such as for use in preclinical testing. In some embodiments, the method involves engineering cells that comprise a TCR or antigen-binding fragment thereof that is chimeric or humanized, such as for use in treatment, diagnostic, or monitoring methods in human subjects.

In some embodiments, the nucleic acid molecule, e.g., the nucleic acid molecule being introduced into the cell, is comprised within a vector, e.g., an expression vector, including any vector described herein, e.g., in Section V.

In some embodiments, the method of producing comprises incubating the cell under conditions to allow for expansion of the cell into a population of engineered cells. In some embodiments, the incubating is carried out in a culture vessel, such as a unit, well, chamber, tube, column, valve, vial, culture dish, or other container for culturing and/or expanding cells. The conditions can include one or more of particular media, oxygen content, temperature, carbon dioxide content, time, agents, e.g., nutrients, antibiotics, amino acids, ions, and/or stimulatory agents or factors, such as cytokines, chemokines, antigens, binding partners, or any other agent that, e.g., can activate the cells. In some embodiments, the incubation is carrier out using one or more techniques as described in U.S. Pat. No. 6,040,177, Klebanoff et al. (2012) J Immunother. 35(9): 651-660, Terakura et al. (2012) Blood. 1:72-82, and Wang et al. (2012) J Immunother. 35(9):689-701.

Also provided herein are methods of engineering a cell, comprising introducing any of the nucleic acid molecules described herein, e.g., any nucleic acid molecule encoding any TCR or antigen-binding fragment thereof, or a Vα region or Vβ region thereof, or any of the vectors described herein, into a cell.

Also provided herein are methods of engineering a cell, comprising: introducing any of the nucleic acid molecules described herein, e.g., any nucleic acid molecule encoding any TCR or antigen-binding fragment thereof, or a Vα region or Vβ region thereof, or any of the vectors described herein, into a cell; and editing and/or disrupting one or more genes endogenous to the cell.

Also provided herein are methods of engineering a cell, comprising: introducing any of the nucleic acid molecules described herein, e.g., any nucleic acid molecule encoding any TCR or antigen-binding fragment thereof, or a Vα region or Vβ region thereof, or any of the vectors described herein, into a cell; and editing and/or disrupting the TRAC gene and/or the TRBC gene.

Also provided herein are methods of engineering a cell, comprising: editing and/or disrupting the endogenous TRAC gene and/or the endogenous TRBC gene in a cell; introducing any of the nucleic acid molecules described herein, e.g., any nucleic acid molecule encoding any TCR or antigen-binding fragment thereof, or a Vα region or Vβ region thereof, or any of the vectors described herein, into the cell, wherein the nucleic acid molecule comprises a first homology arm and a second homology arm that are homologous to the first flanking sequence and the second flanking sequence of one of the one or more endogenous genes, and wherein the nucleic acid sequence encoding the TCR or an antigen-binding fragment thereof, or an alpha or beta chain thereof, is situated between the first homology arm and the second homology arm; and wherein the nucleic acid molecule recombines into the edited and/or disrupted endogenous TRAC gene and/or endogenous TRBC gene.

In some embodiments, the editing and/or disrupting results in a genetic disruption in the one or more endogenous genes. In some embodiments, a genetic disruption includes, e.g., a disruption to the gene that results in a reduction, elimination, or disruption of gene expression, or a deletion or knockout (partial or complete) of the gene endogenous to the cell. In some embodiments, the genetic disruption results in a reduction or elimination of expression of the gene product of the endogenous gene.

In some of any of such embodiments, the method of engineering a cell is a method of engineering a regulatory T (Treg) cell.

In some embodiments, the introducing is performed using any method for introducing a nucleic acid molecule or vector into a cell. In some embodiments, introducing the nucleic acid molecule or vector is carrier out using transfection, e.g., non-viral-based DNA transfection; a transposon-based system, e.g., Sleeping Beauty or PiggyBac; electroporation, e.g., RNA electroporation such as electroporation-mediated mRNA transfection, or DNA electroporation; viral vector systems; non-viral vector systems; or genome editing techniques.

In some embodiments, the introducing is carried out by transfection. In some embodiments, the introducing by transfection comprises introducing any of the vectors described herein, e.g., as described in Section V. In some embodiments, the introducing is carried out by transfection. In some embodiments, the introducing by transfection comprises introducing a vector comprising any of the nucleic acid molecules described herein, e.g., as described in Section V. In some embodiments, the introducing by transfection comprises introducing a vector comprising any of the nucleic acid molecules encoding a TCR or antigen-binding fragment thereof, or an alpha or beta chain thereof, or a Vα region or Vβ region thereof. Transfection involves introducing a foreign, e.g., heterologous, nucleic acid molecule, e.g., DNA, into a eukaryotic cell. Transfection can be stable or transient. Stable transfection typically involves integrating the transfected nucleic acid molecule into the host genome, e.g., to allow for long-term stable expression of the nucleic acid molecule. Transient transfection typically does not involve integration of the nucleic acid molecule into the host genome. In some embodiments, the vector used is the vector as depicted in FIG. 20.

In some embodiments, the introducing by transfection can be carried out by chemical transfection, physical transfection, or viral transfection. In some embodiments, the physical transfection method is or comprises electroporation. In some embodiments, the physical transfection method is or comprises microinjection. In some embodiments, the chemical transfection method is or comprises the use of a chemical agent that facilitates entry of the nucleic acid molecule into the cell, e.g., calcium phosphate, cationic polymers, or liposomes. In some embodiments, the viral transfection comprises the use a virus as a carrier for introducing the nucleic acid molecule into the cell by a method referred to as transduction.

In some embodiments, the introducing is carried out by electroporation.

In some embodiments, the introducing is carried out by transduction, e.g., viral transduction, using a viral vector. In some embodiments, the transduction involves incorporating the nucleic acid molecules into a viral vector.

In some embodiments, the introducing comprises introducing a viral vector comprising the nucleic acid molecule into the cell. In some embodiments, the introducing is carried out by transduction, wherein a viral vector comprises the nucleic acid molecule. In some embodiments, the transduction comprises introducing a viral vector comprising the nucleic acid molecule into the cell. In some embodiments, the viral vector is a retroviral vector, a gammaretroviral vector, a lentiviral vector, or an AAV vector, such as an scAAV vector.

In some embodiments, the introducing is carried out using a genome editing technique. In some embodiments, the editing and/or disrupting is carried out using a genome editing technique. In some embodiments, the genome editing technique involves the use of a nuclease in combination with DNA repair using non-homologous end joining (NHEJ) or homology-directed repair (HDR). Exemplary nucleases that may be used in genome editing techniques for introducing a nucleic acid molecule described herein, e.g., a nucleic acid molecule encoding a TCR or antigen-binding fragment thereof, or an alpha or beta chain thereof, include meganucleases, zinc-finger nucleases, transcription factor-like effector nucleases (TALENs), metaTAL nucleases, and Cas nucleases, such as Cas9, Cas12a, and Cas13. Accordingly, in some embodiments, the introducing is carried out using a genome editing technique involving a nuclease that is a meganuclease, a zinc-finger nuclease, a TALENs, a metaTAL nuclease, or a Cas nuclease, such as Cas9, Cas12a, or Cas13. In some embodiments, the introducing is carried out using a genome editing technique involving a nuclease that is a Cas nuclease, such as Cas9, Cas12a, or Cas13. In some embodiments, the genome editing technique is CRISPR-Cas9.

In some embodiments, the introducing comprises introducing a TCR or antigen-binding fragment thereof as described herein, e.g., in Section III, and is carried out using a genome editing technique that is CRISPR-Cas9 and comprises a vector encoding a single guide RNA (sgRNA). In some embodiments, the editing and/or disrupting is carried out using a genome editing technique that is CRISPR-Cas9 and comprises a vector encoding a single guide RNA (sgRNA). An sgRNA are a combination of two RNA molecules: a tracrRNA that is responsible for endonuclease, e.g., Cas9 endonuclease, activity, and a crRNA that is responsible for targeting the target site of the DNA, e.g., a target site in a target gene of interest, such as an endogenous gene of interest. In some embodiments, the crRNA sequence comprises a nucleic acid sequence set forth in any one of SEQ ID NOs: 18-23. In some embodiments, the crRNA sequence targets a human TRAC gene and comprises a nucleic acid sequence set forth in SEQ ID NO: 22. In some embodiments, the crRNA sequence targets a human TRBC gene and comprises a nucleic acid sequence set forth in SEQ ID NO: 23. In some embodiments, the introducing comprises introducing a crRNA sequence targeting a human TRAC gene that comprises a nucleic acid sequence set forth in SEQ ID NO: 22, and/or comprises introducing a crRNA sequence targeting a human TRBC gene that comprises a nucleic acid sequence set forth in SEQ ID NO: 23. In some embodiments, the crRNA sequence targets a murine TRAC gene and comprises a nucleic acid sequence set forth in SEQ ID NO: 18 or 19. In some embodiments, the crRNA sequence targets a murine TRBC gene and comprises a nucleic acid sequence set forth in SEQ ID NO: 20 or 21. In some embodiments, the introducing comprises introducing a crRNA sequence targeting a murine TRAC gene that comprises a nucleic acid sequence set forth in SEQ ID NO: 18 or 19, and/or comprises introducing a crRNA sequence targeting a murine TRBC gene that comprises a nucleic acid sequence set forth in SEQ ID NO: 20 or 21.

In some embodiments, the nucleic acid molecule further comprises a first homology arm and a second homology arm that are homologous to the first flanking sequence and the second flanking sequence of one of the one or more endogenous genes, wherein the nucleic acid sequence encoding the TCR or an antigen-binding fragment thereof, or an alpha or beta chain thereof, is situated between the first homology arm and the second homology arm.

In some embodiments, the nucleic acid molecule is contained within a vector comprising a first homology arm and a second homology arm that are homologous to the first flanking sequence and the second flanking sequence of one of the one or more endogenous genes, wherein the nucleic acid sequence encoding the TCR or an antigen-binding fragment thereof, or an alpha or beta chain thereof, is situated between the first homology arm and the second homology arm.

In some embodiments, the editing and/or disrupting is carried out by one or more agents capable of editing and/or disrupting one or more genes endogenous to the cell. In some embodiments, the method further comprises introducing into the cell one or more agents capable of editing and/or disrupting one or more genes endogenous to the cell. The one or more agents capable of editing and/or disrupting one or more genes endogenous to the cell are not limited and can include any agent or agents capable of such. In some embodiments, the one or more agents capable of editing and/or disrupting one or more genes endogenous to the cell result in a reduction, elimination, or disruption of gene expression, or a deletion or knockout (partial or complete) of the gene endogenous to the cell. Exemplary methods for disrupting expression of an endogenous TCR or alpha or beta chain thereof are known in the art, e.g., in PCT Publication No. WO 2015/161276, U.S. Publication No. US 2014/0301990, and U.S. Pat. No. 9,273,283.

In some embodiments, the one or more agents capable of editing and/or disrupting one or more genes endogenous to the cell comprise an inhibitory nucleic acid that targets a nucleic acid encoding the endogenous TCR or an alpha or beta chain thereof. In some embodiments, the inhibitory nucleic acid is or comprises a microRNA (miRNA), a short hairpin RNA (shRNA), a microRNA precursor (miRNA precursor), a small interfering RNA (siRNA), or a microRNA-adapted shRNA. In some embodiments, the one or more agents capable of editing and/or disrupting one or more genes endogenous to the cell comprises one or more agents capable of inducing a DNA break.

In some embodiments, the one or more genes endogenous to the cell each comprise a target site, and one or more of the one or more agents specifically bind to or recognizes the target site. The target site is the site, or a portion thereof, within the target gene that is targeted by the one or more agents, e.g., for editing and/or disrupting the target gene. In some embodiments, the one or more genes endogenous to the cell comprises a T Cell Receptor Alpha Constant (TRAC) gene and/or a T Cell Receptor Beta Constant (TRBC) gene.

In some embodiments, the one or more agents comprises a nuclease. In some embodiments, the nuclease specifically binds to or recognizes the target site. The nuclease can be any nuclease suitable for use in editing and/or disrupting a gene. In some embodiments, the nuclease is selected from the group consisting of a meganuclease, a zinc-finger nuclease, a transcription activator-like effector nuclease (TALEN), a megaTAL nuclease, and a clustered regularly interspaced short palindromic repeats (CRISPR)-associated (Cas) nuclease.

In some embodiments, the one or more agents comprise a nuclease based on the Argonaute system (Swarts et al., 2014, Nature, 507(7491): 258-261).

In some embodiments, the nuclease is a Cas nuclease. In some embodiments, the Cas nuclease is a CRISPR-Cas9 nuclease. In some embodiments, the CRISPR-Cas9 system includes an engineered crRNA/tracr RNA (also referred to as a “single guide RNA” to guide the system to specific cleavage, e.g., specific cleavage of a target site of a target gene, such as an endogenous target gene.

In some embodiments, the one or more agents comprises a Cas nuclease and one or more single guide RNA (sgRNA). In some embodiments, each of the one or more sgRNA specifically binds to, hybridizes with, or recognizes a target sequence in one of the one or more endogenous genes. In some embodiments, the sgRNA comprises a sequence for targeting the constant region of the endogenous TCR gene. In some embodiments, the sequence for targeting the constant region of the endogenous TCR gene comprises the nucleic acid sequence of any one of SEQ ID NOs: 18-23. In some embodiments, the one or more single guide RNA is encoded by a nucleic acid or vector that is introduced into the cell. In some embodiments, the sgRNA comprises a crRNA sequence of any one of SEQ ID NOs: 18-23.

In some embodiments, the one or more sgRNA comprises an sgRNA that specifically binds to, hybridizes with, or recognizes a target sequence in an endogenous TRAC gene, and/or comprises an sgRNA that specifically binds to, hybridizes with, or recognizes a target sequence in an endogenous TRBC gene.

In some embodiments, the one or more sgRNA comprises an sgRNA that targets the murine TRAC gene and/or an sgRNA that targets the murine TRBC gene. In some embodiments, the sgRNA targets a murine TRAC gene and comprises the nucleic acid sequence, e.g., crRNA sequence, of SEQ ID NO: 18 or 19. In some embodiments, the sgRNA targets a murine TRBC gene and comprises the nucleic acid sequence, e.g., crRNA sequence, of SEQ ID NO: 20 or 21. In some embodiments, the one or more agents comprises an sgRNA that targets a murine TRAC gene and comprises the nucleic acid sequence, e.g., crRNA sequence, of SEQ ID NO: 18 or 19; and comprises an sgRNA that targets a murine TRBC gene and comprises the nucleic acid sequence, e.g., crRNA sequence, of SEQ ID NO: 20 or 21.

In some embodiments, the one or more sgRNA comprises an sgRNA that targets the human TRAC gene and/or an sgRNA that targets the human TRBC gene. In some embodiments, the sgRNA targets a human TRAC gene and comprises the nucleic acid sequence, e.g., crRNA sequence, of SEQ ID NO: 22. In some embodiments, the sgRNA targets a human TRBC gene and comprises the nucleic acid sequence, e.g., crRNA sequence, of SEQ ID NO: 23. In some embodiments, the one or more agents comprises an sgRNA that targets a human TRAC gene and comprises the nucleic acid sequence, e.g., crRNA sequence, of SEQ ID NO: 22; and comprises an sgRNA that targets a human TRBC gene and comprises the nucleic acid sequence, e.g., crRNA sequence, of SEQ ID NO: 23.

Accordingly, in some embodiments, the endogenous TRAC gene and/or the endogenous TRBC gene is knocked out, eliminated, or disrupted using a gene editing technique, such as CRISPR-Cas9, e.g., using an sgRNA targeting the endogenous TRAC gene and/or the endogenous TRBC gene, e.g., one or more sgRNAs comprising the nucleic acid sequence of any one of SEQ ID NOs: 18-23; and the TCR or antigen-binding fragment thereof is introduced into the cell, e.g., using any of the vectors described herein.

In some embodiments, the method further comprises introducing into the cell one or more agents capable of inserting the nucleic acid molecule into the genome of the cell. In some embodiments, the one or more agents comprises a transposon or a transposon-based system. The transposon or the transposon-based system can be any transposon or transposon-based system suitable for introducing a nucleic acid molecule of interest into the genome of the cell, e.g., for stable expression of the gene of interest. In some embodiments, the transposon comprises a Sleeping Beauty transposon or a PiggyBac transposon, or the transposon-based system comprises a Sleeping Beauty transposon-based system or a PiggyBac transposon-based system.

Also provided herein is a method of engineering a cell, comprising: introducing, into the cell, one or more agents capable of editing and/or disrupting one or more endogenous genes in the cell, wherein each of the one or more endogenous genes comprises a first flanking sequence and a second flanking sequence; and introducing, into the cell, one or more nucleic acid molecules, wherein each of the one or more nucleic acid molecules comprises: (i) a first homology arm and a second homology arm that are homologous to the first flanking sequence and the second flanking sequence of one of the one or more endogenous genes, and (ii) a nucleic acid sequence of interest that is located between the first homology arm and the second homology arm. In some embodiments, the one or more agents capable of editing and/or disrupting one or more endogenous genes in the cell comprises one or more agents capable of inducing a DNA break in one or more endogenous genes in the cell, e.g., the one or more agents is capable of inducing a DNA break in a target site in one or more of the endogenous genes in the cell.

Also provided herein is a method of engineering a cell, comprising: introducing, into the cell, one or more agents capable of inducing a DNA break in one or more endogenous genes in the cell, wherein each of the one or more endogenous genes comprises a first flanking sequence and a second flanking sequence; and introducing, into the cell, one or more nucleic acid molecules, wherein each of the one or more nucleic acid molecules comprises: (i) a first homology arm and a second homology arm that are homologous to the first flanking sequence and the second flanking sequence of one of the one or more endogenous genes, and (ii) a nucleic acid sequence of interest that is located between the first homology arm and the second homology arm.

In some embodiments, the one or more nucleic acid molecules can comprise any nucleic acid molecule described herein, e.g., in Section V, including any nucleic acid molecule encoding any TCR or antigen-binding fragment described herein, e.g., in Section III.

In some embodiments, the nucleic acid sequence of interest encodes a T cell receptor (TCR) or antigen-binding fragment thereof, wherein the TCR or antigen-binding fragment thereof comprises an alpha chain comprising a variable alpha (Vα) region and a beta chain comprising a variable beta (Vβ) region, wherein the Vα region comprises a complementarity determining region 1 (CDR-1), CDR-2, and CDR-3 comprising the amino acid sequences of SEQ ID NOs: 12, 13, and 14, respectively; and the Vβ region comprises a CDR-1, CDR-2, and CDR-3 comprising the amino acid sequences of SEQ ID NOs: 15, 16, and 17, respectively. In some embodiments, the nucleic acid sequence of interest encodes a TCR or antigen-binding fragment thereof comprising a Vα region comprising the amino acid sequence of SEQ ID NO: 5 and a Vβ region comprising the amino acid sequence of SEQ ID NO: 8, or comprises a Vα region comprising an amino acid sequence having at least 90%, 91%, 92%, 93%, 94%, 95%, 96%, 97%, 98%, or 99% sequence identity to SEQ ID NO: 5, and comprises a Vβ region comprising an amino acid sequence having at least 90%, 91%, 92%, 93%, 94%, 95%, 96%, 97%, 98%, or 99% sequence identity to SEQ ID NO: 8. In some embodiments, the nucleic acid sequence of interest encodes a TCR or antigen-binding fragment thereof comprising a Vα region comprising an amino acid sequence having at least 99% sequence identity to SEQ ID NO: 5, and comprises a Vβ region comprising an amino acid sequence having at least 99% sequence identity to SEQ ID NO: 8. In some embodiments, the nucleic acid sequence of interest encodes a TCR or antigen-binding fragment thereof comprising a Vα region comprising the amino acid sequence of SEQ ID NO: 5 and a Vβ region comprising the amino acid sequence of SEQ ID NO: 8.

In some embodiments, the one or more genes endogenous to the cell comprises a TRAC gene and/or a TRBC gene.

The one or more agents capable of inducing a DNA break can be any agent capable of introducing a break in a DNA molecule, e.g., a nuclease. In some embodiments, the one or more agents capable of inducing a DNA break in one or more endogenous genes in the cell comprises a nuclease. In some embodiments, the nuclease is selected from the group consisting of a meganuclease, a zinc-finger nuclease, a TALEN, a megaTAL nuclease, and a Cas nuclease. In some embodiments, the Cas nuclease is a Cas9 nuclease.

In some embodiments, the one or more agents capable of inducing a DNA break comprises a Cas nuclease and one or more single guide RNA (sgRNA). In some embodiments, each of the one or more sgRNA specifically binds to, hybridizes with, or recognizes a target sequence in one of the one or more endogenous genes. In some embodiments, the sgRNA comprises a sequence for targeting the constant region of the endogenous TCR gene. In some embodiments, the sequence for targeting the constant region of the endogenous TCR gene comprises the nucleic acid sequence of any one of SEQ ID NOs: 18-23.

In some embodiments, the one or more sgRNA comprises an sgRNA that specifically binds to, hybridizes with, or recognizes a target sequence in an endogenous TRAC gene, and/or comprises an sgRNA that specifically binds to, hybridizes with, or recognizes a target sequence in an endogenous TRBC gene.

In some embodiments, the one or more sgRNA comprises an sgRNA that targets the murine TRAC gene and/or an sgRNA that targets the murine TRBC gene. In some embodiments, the sgRNA targets a murine TRAC gene and comprises the nucleic acid sequence of SEQ ID NO: 18 or 19. In some embodiments, the sgRNA targets a murine TRBC gene and comprises the nucleic acid sequence of SEQ ID NO: 20 or 21. In some embodiments, the one or more agents comprises an sgRNA that targets a murine TRAC gene and comprises the nucleic acid sequence of SEQ ID NO: 18 or 19; and comprises an sgRNA that targets a murine TRBC gene and comprises the nucleic acid sequence of SEQ ID NO: 20 or 21.

In some embodiments, the one or more sgRNA comprises an sgRNA that targets the human TRAC gene and/or an sgRNA that targets the human TRBC gene. In some embodiments, the sgRNA targets a human TRAC gene and comprises the nucleic acid sequence of SEQ ID NO: 22. In some embodiments, the sgRNA targets a human TRBC gene and comprises the nucleic acid sequence of SEQ ID NO: 23. In some embodiments, the one or more agents comprises an sgRNA that targets a human TRAC gene and comprises the nucleic acid sequence of SEQ ID NO: 22; and comprises an sgRNA that targets a human TRBC gene and comprises the nucleic acid sequence of SEQ ID NO: 23.

In some embodiments, the method results in reduced or eliminated expression of the one or more endogenous genes; and/or introduces expression of the TCR or antigen-binding fragment thereof in the cell. In some embodiments, the method results in reduced or eliminated expression of the one or more endogenous genes and incorporation of a nucleic acid molecule encoding the TCR or antigen-binding fragment thereof into a target site contained within the one or more endogenous genes.

In some embodiments, the one or more nucleic acid molecules is comprised within a vector, such as any of the vectors described herein, e.g., in Section V. In some embodiments, the vector is a donor vector, e.g., a donor plasmid for gene editing.

In some embodiments, the introducing of one or more of: (i) the one or more agents capable of editing and/or disrupting one or more endogenous genes in the cell, (ii) one or more agents capable of inducing a DNA break in one or more endogenous genes in the cell, and/or (iii) one or more nucleic acid molecules, is carried out by transfection, electroporation, or transduction, or any combination thereof.

In some embodiments, the cell is from a subject having Alzheimer's Disease.

In some embodiments, the cell is from a donor subject. In some embodiments, the donor subject does not have Alzheimer's Disease or has not been diagnosed as having Alzheimer's Disease.

In some embodiments, the cell is an immune cell. In some embodiments, the immune cell is a T cell, a B cell, or a Natural Killer (NK) cell. In some embodiments, the cell is a T cell, a B cell, or a Natural Killer (NK) cell. In some embodiments, the cell is a T cell. In some embodiments, the T cell is a regulatory T (Treg) cell or an effector T cell. In some embodiments, the T cell is a regulatory T (Treg) cell. Accordingly, in some embodiments, the cell is a T cell, such as a Treg cell. In some embodiments, the cell is a Treg cell. In some embodiments, the cell is a CD4+ and/or CD8+ T cell. In some embodiments, the cell is a CD4+ T cell. In some embodiments, the cell is a CD4+/CD25+/FOXP3+ T cell.

### VII. COMPOSITIONS

Also provided herein are compositions, e.g., pharmaceutical compositions, comprising any TCR or antigen-binding fragment thereof as described herein, e.g., in Section III, any conjugate as described herein, e.g., in Section III, any engineered cell as described herein, e.g., in Section III, any nucleic acid molecule or vector described herein, e.g., in Section V, or any engineered cell produced or engineered by any method as described herein, e.g., in Section VI; and, optionally, a pharmaceutically acceptable excipient or carrier.

In some embodiments, the composition is a pharmaceutical composition comprising any of the engineered cells described herein, and a pharmaceutically acceptable excipient or carrier. A “pharmaceutically acceptable carrier” refers to an ingredient in a pharmaceutical formulation or composition, other than an active ingredient, which is nontoxic to a subject. A pharmaceutically acceptable carrier includes, but is not limited to, a buffer, excipient, a preservative, or a stabilizer. Exemplary carriers are described in, e.g., Remington's Pharmaceutical Sciences 16th Edition, Osol. A. Ed. (1980).

In some embodiments, the composition, e.g., pharmaceutical composition, comprises the TCR or antigen-binding fragment thereof, the conjugate, and/or the engineered cell in amounts effective to treat, prevent, or delay the disease or condition, e.g., Alzheimer's Disease, such as a therapeutically effective or prophylactically effective amount. In some embodiments, the composition, e.g., pharmaceutical composition, comprises the TCR or antigen-binding fragment thereof, the conjugate, and/or the engineered cell in amounts effective to diagnose and/or monitor progression of a disease or condition, e.g., Alzheimer's Disease.

### VIII. METHODS AND USES OF ENGINEERED CELLS AND COMPOSITIONS THEREOF

Provided herein are methods and uses, e.g., of treatment, diagnosis, or monitoring progression, that involves the administration or use of any of the engineered cells or compositions as described herein, e.g., in Section IV or VII.

Provided herein is a method of treatment, e.g., a method of treating a disease or condition associated with amyloid beta, such as the treatment of Alzheimer's Disease, comprising administering of any of the engineered cells, e.g., engineered T cells, such as Treg cells, described herein, to a subject having Alzheimer's Disease.

Provided herein is a method of treatment, e.g., a method of treating a disease or condition associated with amyloid beta, such as the treatment of Alzheimer's Disease, comprising administering of any of the engineered cells, e.g., engineered T cells, such as Treg cells, described herein, to a subject having a disease or condition associated with or caused in whole or in part by amyloid beta.

Also provided herein is a method of treatment, e.g., a method of treating a disease or condition associated with amyloid beta, such as the treatment of Alzheimer's Disease, comprising administering any of the compositions, e.g., pharmaceutical compositions, described herein, to a subject having Alzheimer's Disease.

Also provided herein is a method of treatment, e.g., a method of treating a disease or condition associated with amyloid beta, such as the treatment of Alzheimer's Disease, comprising administering any of the compositions, e.g., pharmaceutical compositions, described herein, to a subject having a disease or condition associated with or caused in whole or in part by amyloid beta.

Also provided herein is a method of treatment of a disease or condition associated with amyloid beta, e.g., Alzheimer's Disease, comprising administering any of the compositions, e.g., pharmaceutical compositions, described herein, or any of the engineered cells, e.g., engineered T cells, such as Treg cells, described herein, to a subject having a disease or condition, e.g., a disease or condition associated with or caused in whole or in part by amyloid beta.

Also provided herein is a method of treatment of Alzheimer's Disease, comprising: engineering a cell by any of the methods described herein, e.g., in Section VI; culturing the engineered cells under conditions to produce a population of the engineered cells; and administering a therapeutically effective amount of the population of engineered cells to a subject having a disease or condition associated with or caused in whole or in part by amyloid beta.

Also provided herein is a method of treatment of Alzheimer's Disease, comprising: engineering a cell by any of the methods described herein, e.g., in Section VI; culturing the engineered cells under conditions to produce a population of the engineered cells; and administering a therapeutically effective amount of the population of engineered cells to a subject having Alzheimer's Disease.

Also provided herein are compositions, e.g., any composition described herein, e.g., in Section VII, for use in treating Alzheimer's Disease, e.g., in a subject having Alzheimer's Disease.

Also provided herein are uses of any of the compositions described herein, e.g., in Section VII, for treating Alzheimer's Disease, e.g., in a subject having Alzheimer's Disease.

Also provided herein are uses of any of the compositions described herein, e.g., in Section VII, for the manufacture of a medicament for treating Alzheimer's Disease, e.g., in a subject having Alzheimer's Disease.

In some embodiments, the disease or condition associated with or caused in whole or in part by amyloid beta is Alzheimer's Disease.

In some embodiments, the subject is a human, e.g., a human having Alzheimer's Disease.

In some embodiments, the cell is from a subject having a disease or condition associated with amyloid beta or caused in whole or in part by amyloid beta, such as Alzheimer's Disease. In some embodiments, the cell is from the subject having a disease or condition associated with amyloid beta or caused in whole or in part by amyloid beta, such as Alzheimer's Disease, e.g., the subject to which the population of engineered cells is to be administered. As such, in some embodiments, the cell is autologous to the subject. In some embodiments, the cell is allogeneic to the subject. In some embodiments, the cell is from a donor subject. In some embodiments, the donor subject does not have Alzheimer's Disease or has not been diagnosed as having Alzheimer's Disease. In some embodiments, the donor subject is not the subject to which the population of engineered cells is to be administered.

In some embodiments, the cell is an immune cell. In some embodiments, the immune cell is a T cell, a B cell, or a Natural Killer (NK) cell. In some embodiments, the cell is a T cell, a B cell, or a Natural Killer (NK) cell. In some embodiments, the cell is a T cell. In some embodiments, the T cell is a regulatory T (Treg) cell or an effector T cell. In some embodiments, the T cell is a regulatory T (Treg) cell. Accordingly, in some embodiments, the cell is a T cell, such as a Treg cell. In some embodiments, the cell is a Treg cell. In some embodiments, the cell is a CD4+ and/or CD8+ T cell. In some embodiments, the cell is a CD4+ T cell. In some embodiments, the cell is a CD4+/CD25+/FOXP3+ T cell.

Also provided herein are methods of diagnosing a disease or condition associated with amyloid beta or caused in whole or in part by amyloid beta, comprising administering any TCR or antigen-binding fragment thereof as described herein, e.g., in Section III, or any conjugate as described herein, e.g., in Section III, to a subject having or suspected of having a disease or condition associated with amyloid beta or caused in whole or in part by amyloid beta. In some embodiments, the subject expresses amyloid beta, e.g., human amyloid beta, at levels higher than in a healthy subject.

Also provided herein are methods of diagnosing Alzheimer's Disease, comprising administering any TCR or antigen-binding fragment thereof as described herein, e.g., in Section III, or any conjugate as described herein, e.g., in Section III, to a subject having or suspected of having Alzheimer's Disease. In some embodiments, the subject expresses amyloid beta, e.g., human amyloid beta, at levels higher than in a healthy subject.

In some embodiments, the method of diagnosis further comprises detecting the level or absence of binding between the TCR or antigen-binding fragment thereof or the conjugate with amyloid beta, and, optionally, comparing the level or absence of binding to the level or absence of binding between the TCR or antigen-binding fragment thereof or the conjugate with amyloid beta as detected as one or more preceding time points. In some embodiments, the method further comprises identifying the subject as having a disease or condition associated with amyloid beta, e.g., Alzheimer's Disease, if the level of binding exceeds a threshold level.

Also provided herein are methods of diagnosing a disease or condition associated with amyloid beta or caused in whole or in part by amyloid beta, comprising administering any engineered cell, e.g., as described in Section IV, comprising any TCR or antigen-binding fragment thereof as described herein, e.g., in Section III, or any conjugate as described herein, e.g., in Section III, to a subject having or suspected of having a disease or condition associated with amyloid beta or caused in whole or in part by amyloid beta; and detecting the level of administered engineered cells at one or more subsequent time points. In some embodiments, the subject expresses amyloid beta, e.g., human amyloid beta, at levels higher than in a healthy subject. In some embodiments, the one or more subsequent time points can be any one or more subsequent time points, such as at or about or at least at or about 1 hour, 3 hours, 6 hours, 9 hours, 12 hours, 24 hours, 2 days, 3 days, 4 days, 5 days, 6 days, 1 week, 2 weeks, 3 weeks, 4 weeks, 2 months, 3 months, 4 months, 5 months, or 6 months following administration of the engineered cells.

Also provided herein are methods of diagnosing Alzheimer's Disease, comprising administering any engineered cell, e.g., as described in Section IV, comprising any TCR or antigen-binding fragment thereof as described herein, e.g., in Section III, or any conjugate as described herein, e.g., in Section III, to a subject having or suspected of having Alzheimer's Disease. In some embodiments, the subject expresses amyloid beta, e.g., human amyloid beta, at levels higher than in a healthy subject. In some embodiments, the one or more subsequent time points can be any one or more subsequent time points, such as at or about or at least at or about 1 hour, 3 hours, 6 hours, 9 hours, 12 hours, 24 hours, 2 days, 3 days, 4 days, 5 days, 6 days, 1 week, 2 weeks, 3 weeks, 4 weeks, 2 months, 3 months, 4 months, 5 months, or 6 months following administration of the engineered cells.

Patients with Alzheimer's Disease have clonal expansion of the T cells in the peripheral blood, i.e., two or more cells of the same TCR sequence that can be reactive against amyloid beta. As such, it is envisioned that the engineered cells provided herein that express the amyloid beta-specific TCR or antigen-binding fragment thereof as described herein can be used as a biosensor to detect pathogenic amyloid beta or antigen-presenting cells (APCs) or innate immune cells that present pathogenic peptides of amyloid beta. As such, identification of cells, e.g., T cells, such as Treg cells, with the TCR sequences of the TCR or antigen-binding fragment thereof provided herein, e.g., in Section III, can be used as a biomarker for the diagnosis of a disease or condition associated with amyloid beta, such as Alzheimer's Disease.

In some embodiments, the method of diagnosis further comprises detecting the number or absence of engineered cells comprising the TCR or antigen-binding fragment thereof or the conjugate, and, optionally, comparing the number of absence of engineered cells as detected as one or more preceding time points. In some embodiments, the method further comprises identifying the subject as having a disease or condition associated with amyloid beta, e.g., Alzheimer's Disease, if the number of engineered cells exceeds a threshold level.

Also provided herein are methods comprising the use of the engineered cells, TCRs or antigen-binding fragments thereof, and conjugates, as described herein in monitoring disease progression or response to therapy, e.g., any of the treatments described herein. Clinically, Alzheimer's Disease (AD) and other neurodegenerative disorders (for example, Parkinson's disease) emerging evidence demonstrates that there are parallel effector T cell responses (Th1 and Th17) patterns of immune cells and cytokine production that emerge as a consequence of amyloid-beta specific TCRs and associated with inflammation and progressive neurodegeneration. The parallels between such T cell responses and progressive disease would not be solely limited for staging of neurodegenerative responses, but also used in response to therapy. As such it is also becoming increasing clear that an autoimmune component of AD and PD is emerging and although the disease itself is of unknown etiology the role of T cells in neuronal degeneration is clear and substantiated. Although the interface between innate (microglia) and T cell (effector cell) activities is a complex process the interaction between various cytokines, chemokines, signaling molecules, and immune pathways will provide important insights into staging of disease and ultimately used for therapeutic monitoring and targets for AD.

Accordingly, also provided herein are methods of monitoring the progression of a disease or condition associated with amyloid beta or caused in whole or in part by amyloid beta, administering any TCR or antigen-binding fragment thereof as described herein, e.g., in Section III, or any conjugate as described herein, e.g., in Section III, to a subject having or suspected of having a disease or condition associated with amyloid beta or caused in whole or in part by amyloid beta. In some embodiments, the method of monitoring the progression of a disease or condition associated with amyloid beta or caused in whole or in part by amyloid beta further comprises detecting the level or absence of binding between the TCR or antigen-binding fragment thereof or the conjugate with amyloid beta, and, optionally, comparing the level or absence of binding to the level or absence of binding between the TCR or antigen-binding fragment thereof or the conjugate with amyloid beta as detected as one or more preceding time points.

Also provided herein are methods of diagnosing Alzheimer's Disease, comprising administering any TCR or antigen-binding fragment thereof as described herein, e.g., in Section III, or any conjugate as described herein, e.g., in Section III, to a subject having or suspected of having Alzheimer's Disease.

Also provided herein are methods of monitoring the progression of Alzheimer's Disease, administering any TCR or antigen-binding fragment thereof as described herein, e.g., in Section III, or any conjugate as described herein, e.g., in Section III, to a subject having or suspected of having Alzheimer's Disease. In some embodiments, the method of monitoring the progression of Alzheimer's Disease further comprises detecting the level or absence of binding between the TCR or antigen-binding fragment thereof or the conjugate with amyloid beta, and, optionally, comparing the level or absence of binding to the level or absence of binding between the TCR or antigen-binding fragment thereof or the conjugate with amyloid beta as detected as one or more preceding time points.

### IX. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the term “humanized” in the context of a TCR or antigen-binding fragment thereof is a TCR or antigen-binding fragment thereof in which all or substantially all of the CDR residues are derived from non-human CDRs and all or substantially all framework region amino acid residues are derived from human framework regions. A humanized TCR or antigen-binding fragment thereof may optionally include at least a portion of a constant region derived from a human TCR. As used herein, a “humanized” form of a TCR or antigen-binding fragment thereof refers to a modified variant of a non-human TCR or antigen-binding fragment thereof that has undergone humanization, which is typically performed to reduce immunogenicity in humans, while at the same time retaining the specificity and affinity of the parental (non-modified) non-human TCR or antigen-binding fragment thereof. In some embodiments, humanization of a TCR or antigen-binding fragment thereof involves further substituting some framework region residues of a humanized TCR or antigen-binding fragment thereof with corresponding responding residues from a non-human TCR or antigen-binding fragment thereof, such as the TCR or antigen-binding fragment thereof from which the CDRs are derived, e.g., to restore or improve the specificity and/or affinity of the humanized TCR or antigen-binding fragment thereof.

As used herein, the term “chimeric” refers to any nucleic acid sequence and/or amino acid sequence that contains portions from two different sources, e.g., a non-human source and a human source. In the context of a TCR or antigen-binding fragment thereof, a chimeric TCR or antigen-binding fragment thereof can include a Vα region and/or Vβ region derived from a non-human source, e.g., mouse, and a constant domain, e.g., a Cα domain and/or a Cβ region, derived from a human source.

As used herein, “heterologous” in the context of a nucleic acid sequence and/or an amino acid sequence, e.g., encoding or of a TCR or antigen-binding fragment thereof, in a cell, refers to a nucleic acid sequence and/or an amino acid sequence that is not normally present in the genome of that cell. In the context of an engineered cell, a heterologous sequence contained therein refers to a sequence that was introduced into the cell that otherwise was not previously present in that cell.

As used herein, “percent (%) sequence identity” and “percent identity” when used with respect to an amino acid sequence (reference amino acid sequence) is defined as the percentage of amino acid residues in a particular sequence (e.g., a TCR or antigen-binding fragment thereof, such as the alpha chain, beta chain, or a variable region thereof) that are identical with the amino acid residues in the reference amino acid sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

As used herein, an “isolated” nucleic acid molecule refers to a nucleic acid molecule that has been separated from a component of its natural environment. An isolated nucleic acid molecule includes a nucleic acid molecule contained in cells that ordinarily contain the nucleic acid molecule, but the nucleic acid molecule is present extrachromosomally or at a chromosomal location that differs from its natural chromosomal location.

As used herein, a “regulatory T cell” or “Treg cell” refers to a T cell having the phenotype of CD4+/CD25+/FOXP3+. Upon activation, Treg cells secrete immunosuppressive cytokines and chemokines.

As used herein, a disease or condition “associated with amyloid beta” is a disease, condition, or disorder expressing or associated with amyloid beta, e.g., involves cells expressing or specifically expressing amyloid beta, such as at levels higher than in a healthy subject, which contributes, at least partially, to the pathology of the disease or condition. In some embodiments, a disease or condition “associated with amyloid beta” is a disease, condition, or disorder in which there is a buildup of amyloid beta in a brain or a compartment, region, or portion thereof.

As used herein, the singular forms “a,” “an,” and “the” include plural referents unless the context clearly indicates otherwise. For example, “a” or “an” means “at least one” or “one or more.” It is understood that aspects and variations described herein include “consisting” and/or “consisting essentially of” aspects and variations.

### X. EXEMPLARY EMBODIMENTS

Among the provided embodiments are:

1. An engineered cell, comprising a heterologous T cell receptor (TCR) or antigen-binding fragment thereof,
wherein the heterologous TCR or antigen-binding fragment thereof comprises an alpha chain comprising a variable alpha (Vα) region and a beta chain comprising a variable beta (Vβ) region, and
wherein the Vα region comprises a complementarity determining region 1 (CDR-1), CDR-2, and CDR-3 comprising the amino acid sequences of SEQ ID NOs: 12, 13, and 14, respectively; and the Vβ region comprises a CDR-1, CDR-2, and CDR-3 comprising the amino acid sequences of SEQ ID NOs: 15, 16, and 17, respectively.
   2. An engineered cell, comprising a heterologous T cell receptor (TCR) or antigen-binding fragment thereof,
wherein the heterologous TCR or antigen-binding fragment thereof comprises an alpha chain comprising a variable alpha (Vα) region and a beta chain comprising a variable beta (Vβ) region, and
wherein the Vα region comprises a complementarity determining region 1 (CDR-1), CDR-2, and CDR-3 comprising the CDR-1, CDR-2, and CDR-3 amino acid sequences contained within the amino acid sequence of SEQ ID NO: 2 or 5; and the Vβ region comprises a CDR-1, CDR-2, and CDR-3 comprising the CDR-1, CDR-2, and CDR-3 amino acid sequences contained within the amino acid sequence of SEQ ID NO: 4 or 8.
   3. The engineered cell of embodiment 1 or embodiment 2, further comprising a genetic disruption in one or more genes endogenous to the cell.
   4. The engineered cell of embodiment 3, wherein the one or more genes endogenous to the cell comprises a T Cell Receptor Alpha Constant (TRAC) gene and/or a T Cell Receptor Beta Constant (TRBC) gene.
   5. The engineered cell of any one of embodiments 1-4, wherein the engineered cell is a T cell, a B cell, or a Natural Killer (NK) cell.
   6. The engineered cell of any one of embodiments 1-5, wherein the engineered cell is a T cell.
   7. The engineered cell of embodiment 6, wherein the T cell is a regulatory T cell or an effector T cell.
   8. The method of embodiment 6 or embodiment 7, wherein the T cell is a regulatory T (Treg) cell.
   9. The engineered cell of any one of embodiments 1-8, further comprising a nucleic acid molecule encoding the TCR or antigen-binding fragment thereof.
   10. The engineered cell of embodiment 9, wherein the nucleic acid molecule is under the control of one or more endogenous gene promoters.
   11. The engineered cell of embodiment 10, wherein the one or more endogenous gene promoters comprises the endogenous TRAC gene promoter and/or the endogenous TRBC gene promoter.
   12. The engineered cell of embodiment 9 or embodiment 11, wherein the nucleic acid molecule is under the control of an exogenous promoter.
   13. The engineered cell of any one of embodiments 9-12, wherein the nucleic acid molecule is inserted into the endogenous TRAC gene and/or the endogenous TRBC gene.
   14. An engineered cell, comprising:
a heterologous T cell receptor (TCR) or antigen-binding fragment thereof, wherein the heterologous TCR or antigen-binding fragment thereof comprises an alpha chain comprising a variable alpha (Vα) region and a beta chain comprising a variable beta (Vβ) region, and wherein the Vα region comprises a complementarity determining region 1 (CDR-1), CDR-2, and CDR-3 comprising the amino acid sequences of SEQ ID NOs: 12, 13, and 14, respectively; and the Vβ region comprises a CDR-1, CDR-2, and CDR-3 comprising the amino acid sequences of SEQ ID NOs: 15, 16, and 17, respectively; and
a genetic disruption in a T Cell Receptor Alpha Constant (TRAC) gene and/or a T Cell Receptor Beta Constant (TRBC) gene.
   15. An engineered cell, comprising:
a heterologous T cell receptor (TCR) or antigen-binding fragment thereof, wherein the heterologous TCR or antigen-binding fragment thereof comprises an alpha chain comprising a variable alpha (Vα) region and a beta chain comprising a variable beta (Vβ) region, and wherein the Vα region comprises a complementarity determining region 1 (CDR-1), CDR-2, and CDR-3 comprising the CDR-1, CDR-2, and CDR-3 amino acid sequences contained within the amino acid sequence of SEQ ID NO: 2 or 5; and the Vβ region comprises a CDR-1, CDR-2, and CDR-3 comprising the CDR-1, CDR-2, and CDR-3 amino acid sequences contained within the amino acid sequence of SEQ ID NO: 4 or 8; and a genetic disruption in a T Cell Receptor Alpha Constant (TRAC) gene and/or a T Cell Receptor Beta Constant (TRBC) gene. 16. An engineered regulatory T (Treg) cell, comprising:
a heterologous T cell receptor (TCR) or antigen-binding fragment thereof, wherein the heterologous TCR or antigen-binding fragment thereof comprises an alpha chain comprising a variable alpha (Vα) region and a beta chain comprising a variable beta (Vβ) region, and wherein the Vα region comprises a complementarity determining region 1 (CDR-1), CDR-2, and CDR-3 comprising the amino acid sequences of SEQ ID NOs: 12, 13, and 14, respectively; and the Vβ region comprises a CDR-1, CDR-2, and CDR-3 comprising the amino acid sequences of SEQ ID NOs: 15, 16, and 17, respectively; and
a genetic disruption in a T Cell Receptor Alpha Constant (TRAC) gene and/or a T Cell Receptor Beta Constant (TRBC) gene.
   17. An engineered regulatory T (Treg) cell, comprising:
a heterologous T cell receptor (TCR) or antigen-binding fragment thereof, wherein the heterologous TCR or antigen-binding fragment thereof comprises an alpha chain comprising a variable alpha (Vα) region and a beta chain comprising a variable beta (Vβ) region, and wherein the Vα region comprises a complementarity determining region 1 (CDR-1), CDR-2, and CDR-3 comprising the CDR-1, CDR-2, and CDR-3 amino acid sequences contained within the amino acid sequence of SEQ ID NO: 2 or 5; and the Vβ region comprises a CDR-1, CDR-2, and CDR-3 comprising the CDR-1, CDR-2, and CDR-3 amino acid sequences contained within the amino acid sequence of SEQ ID NO: 4 or 8; and
a genetic disruption in a T Cell Receptor Alpha Constant (TRAC) gene and/or a T Cell Receptor Beta Constant (TRBC) gene.
   18. An engineered cell, comprising:
a nucleic molecule encoding a heterologous T cell receptor (TCR) or antigen-binding fragment thereof, wherein the heterologous TCR or antigen-binding fragment thereof comprises an alpha chain comprising a variable alpha (Vα) region and a beta chain comprising a variable beta (Vβ) region, and wherein the Vα region comprises a complementarity determining region 1 (CDR-1), CDR-2, and CDR-3 comprising the amino acid sequences of SEQ ID NOs: 12, 13, and 14, respectively; and the Vβ region comprises a CDR-1, CDR-2, and CDR-3 comprising the amino acid sequences of SEQ ID NOs: 15, 16, and 17, respectively; and
a genetic disruption in a T Cell Receptor Alpha Constant (TRAC) gene and/or a T Cell Receptor Beta Constant (TRBC) gene.
   19. An engineered cell, comprising:
a nucleic acid molecule encoding a heterologous T cell receptor (TCR) or antigen-binding fragment thereof, wherein the heterologous TCR or antigen-binding fragment thereof comprises an alpha chain comprising a variable alpha (Vα) region and a beta chain comprising a variable beta (Vβ) region, and wherein the Vα region comprises a complementarity determining region 1 (CDR-1), CDR-2, and CDR-3 comprising the CDR-1, CDR-2, and CDR-3 amino acid sequences contained within the amino acid sequence of SEQ ID NO: 2 or 5; and the Vβ region comprises a CDR-1, CDR-2, and CDR-3 comprising the CDR-1, CDR-2, and CDR-3 amino acid sequences contained within the amino acid sequence of SEQ ID NO: 4 or 8; and a genetic disruption in a T Cell Receptor Alpha Constant (TRAC) gene and/or a T Cell Receptor Beta Constant (TRBC) gene.
   20. The engineered cell of any one of embodiments 15-17, further comprising a nucleic acid molecule encoding the TCR or antigen-binding fragment thereof.
   21. The engineered cell of any one of embodiments 18-20, wherein the nucleic acid molecule is under the control of one or more endogenous gene promoters.
   22. The engineered cell of embodiment 21, wherein the one or more endogenous gene promoters comprises the endogenous TRAC gene promoter and/or the endogenous TRBC gene promoter.
   23. The engineered cell of any one of embodiments 18-22, wherein the nucleic acid molecule is under the control of an exogenous promoter.
   24. The engineered cell of any one of embodiments 18-23, wherein the nucleic acid molecule is inserted into the endogenous TRAC gene and/or the endogenous TRBC gene.
   25. The engineered cell of any one of embodiments 9-11, 13, 18-22, and 24, wherein the nucleic acid molecule is under the control of the endogenous TRAC gene promoter and/or the endogenous TRBC gene promoter.
   26. The engineered cell of any one of embodiments 3-25, wherein the genetic disruption results in a reduction or elimination of expression of the gene product of the endogenous gene.
   27. The engineered cell of any one of embodiments 1-26, wherein:
the Vα region comprises a CDR-1, CDR-2, and CDR-3 comprising the amino acid sequences of SEQ ID NOs: 12, 13, and 14, respectively; and the Vβ region comprises a CDR-1, CDR-2, and CDR-3 comprising the amino acid sequences of SEQ ID NOs: 15, 16, and 17, respectively; or
the Vα region comprises a CDR-1, CDR-2, and CDR-3 comprising the CDR-1, CDR-2, and CDR-3 amino acid sequences contained within the amino acid sequence of SEQ ID NO: 2 or 5; and the Vβ region comprises a CDR-1, CDR-2, and CDR-3 comprising the CDR-1, CDR-2, and CDR-3 amino acid sequences contained within the amino acid sequence of SEQ ID NO: 4 or 8.
   28. The engineered cell of any one of embodiments 1-27, wherein the Vα region comprises the amino acid sequence of SEQ ID NO: 5 or an amino acid sequence having at least 80%, 85%, 90%, 91%, 92%, 93%, 94%, 95%, 96%, 97%, 98%, or 99% identity to SEQ ID NO: 5; and/or the Vβ comprises the amino acid sequence of SEQ ID NO: 8 or an amino acid sequence having at least 80%, 85%, 90%, 91%, 92%, 93%, 94%, 95%, 96%, 97%, 98%, or 99% identity to the amino acid sequence of SEQ ID NO: 8.
   29. The engineered cell of any one of embodiments 1-28, wherein the Vα region comprises an amino acid sequence of SEQ ID NO: 5 and/or the Vβ region comprises the amino acid sequence of SEQ ID NO: 8.
   30. The engineered cell of any one of embodiments 1-29, wherein the Vα region comprises an amino acid sequence of SEQ ID NO: 5 and the Vβ region comprises the amino acid sequence of SEQ ID NO: 8.
   31. The engineered cell of any one of embodiments 1-30, wherein the TCR or antigen-binding fragment thereof specifically binds to human amyloid beta.
   32. The engineered cell of any one of embodiments 1-31, wherein the alpha chain further comprises an alpha constant (Cα) region and/or the beta chain further comprises a beta constant (Cβ) region.
   33. The engineered cell of embodiment 32, wherein the Cα region comprises the amino acid sequence of SEQ ID NO: 7, or an amino acid sequence having at least 80%, 85%, 86%, 87%, 88%, 89%, 90%, 91%, 92%, 93%, 94%, 95%, 96%, 97%, 98%, or 99% sequence identity to the amino acid sequence of SEQ ID NO: 7; and/or the Cβ region comprises the amino acid sequence of SEQ ID NO: 11, or an amino acid sequence having at least 80%, 85%, 86%, 87%, 88%, 89%, 90%, 91%, 92%, 93%, 94%, 95%, 96%, 97%, 98%, or 99% sequence identity to the amino acid sequence of SEQ ID NO: 11.
   34. The engineered cell of embodiment 32, wherein the Cα region comprises the amino acid sequence of SEQ ID NO: 7, or an amino acid sequence having at least 80%, 85%, 86%, 87%, 88%, 89%, 90%, 91%, 92%, 93%, 94%, 95%, 96%, 97%, 98%, or 99% sequence identity to the amino acid sequence of SEQ ID NO: 7; and the Cβ region comprises the amino acid sequence of SEQ ID NO: 11, or an amino acid sequence having at least 80%, 85%, 86%, 87%, 88%, 89%, 90%, 91%, 92%, 93%, 94%, 95%, 96%, 97%, 98%, or 99% sequence identity to the amino acid sequence of SEQ ID NO: 11.
   35. The engineered cell of embodiment 32, wherein the Cα region comprises the amino acid sequence of SEQ ID NO: 7; and/or the Cβ region comprises the amino acid sequence of SEQ ID NO: 11.
   36. The engineered cell of any one of embodiments 1-35, wherein the alpha chain comprises the amino acid sequence of SEQ ID NO: 2 or an amino acid sequence having at least 80%, 85%, 90%, or 95% identity to SEQ ID NO: 2; and/or the beta chain comprises the amino acid sequence of SEQ ID NO: 4 or an amino acid sequence having at least 80%, 85%, 90%, or 95% identity to SEQ ID NO: 4.
   37. The engineered cell of any one of embodiments 1-36, that is chimeric.
   38. The engineered cell of any one of embodiments 1-28 and 31-36, that is humanized.
   39. The engineered cell of any one of embodiments 1-36, that is murine.
   40. The engineered cell of any one of embodiments 14, 15, and 18-39, wherein the engineered cell is a T cell, a B cell, or a Natural Killer (NK) cell.
   41. The engineered cell of any one of embodiments 14, 15, and 18-40, wherein the engineered cell is a T cell.
   42. The engineered cell of embodiment 41, wherein the T cell is a regulatory T cell or an effector T cell.
   43. The engineered cell of embodiment 41 or embodiment 42, wherein the T cell is a regulatory T (Treg) cell.
   44. A T cell receptor (TCR) or antigen-binding fragment thereof, comprising an alpha chain comprising a variable alpha (Vα) region and a beta chain comprising a variable beta (Vβ) region, wherein the Vα region comprises a complementarity determining region 1 (CDR-1), CDR-2, and CDR-3 comprising the amino acid sequences of SEQ ID NOs: 12, 13, and 14, respectively; and the Vβ region comprises a CDR-1, CDR-2, and CDR-3 comprising the amino acid sequences of SEQ ID NOs: 15, 16, and 17, respectively.
   45. A T cell receptor (TCR) or antigen-binding fragment thereof, comprising an alpha chain comprising a variable alpha (Vα) region and a beta chain comprising a variable beta (Vβ) region, wherein the Vα region comprises a complementarity determining region 1 (CDR-1), CDR-2, and CDR-3 comprising the CDR-1, CDR-2, and CDR-3 amino acid sequences contained within the amino acid sequence of SEQ ID NO: 2 or 5; and the Vβ region comprises a CDR-1, CDR-2, and CDR-3 comprising the CDR-1, CDR-2, and CDR-3 amino acid sequences contained within the amino acid sequence of SEQ ID NO: 4 or 8.
   46. The TCR or antigen-binding fragment thereof of embodiment 37 or embodiment 38, wherein the Vα region comprises a CDR-1, CDR-2, and CDR-3 comprising the CDR-1, CDR-2, and CDR-3 amino acid sequences contained within the amino acid sequence of SEQ ID NO: 2; and the Vβ region comprises a CDR-1, CDR-2, and CDR-3 comprising the CDR-1, CDR-2, and CDR-3 amino acid sequences contained within the amino acid sequence of SEQ ID NO: 4.
   47. The TCR or antigen-binding fragment thereof of embodiment 44 or embodiment 45, wherein the Vα region comprises a CDR-1, CDR-2, and CDR-3 comprising the CDR-1, CDR-2, and CDR-3 amino acid sequences contained within the amino acid sequence of SEQ ID NO: 5; and the Vβ region comprises a CDR-1, CDR-2, and CDR-3 comprising the CDR-1, CDR-2, and CDR-3 amino acid sequences contained within the amino acid sequence of SEQ ID NO: 8.
   48. The TCR or antigen-binding fragment thereof of any one of embodiments 44-47, wherein the Vα region comprises the amino acid sequence of SEQ ID NO: 5 or an amino acid sequence having at least 80%, 85%, 90%, 91%, 92%, 93%, 94%, 95%, 96%, 97%, 98%, or 99% identity to SEQ ID NO: 5; and/or wherein the Vβ comprises the amino acid sequence of SEQ ID NO: 8 or an amino acid sequence having at least 80%, 85%, 90%, 91%, 92%, 93%, 94%, 95%, 96%, 97%, 98%, or 99% identity to the amino acid sequence of SEQ ID NO: 8.
   49. The TCR or antigen-binding fragment thereof of any one of embodiments 44-48, wherein the Vα region comprises an amino acid sequence of SEQ ID NO: 5 and/or the Vβ region comprises the amino acid sequence of SEQ ID NO: 8.
   50. The TCR or antigen-binding fragment thereof of any one of embodiments 44-49, wherein the Vα region comprises an amino acid sequence of SEQ ID NO: 5 and the Vβ region comprises the amino acid sequence of SEQ ID NO: 8.
   51. The TCR or antigen-binding fragment thereof of any one of embodiments 44-50, wherein the TCR or antigen-binding fragment thereof specifically binds to amyloid beta.
   52. The TCR or antigen-binding fragment thereof of any one of embodiments 44-51, wherein the TCR or antigen-binding fragment thereof specifically binds to human amyloid beta.
   53. The TCR or antigen-binding fragment thereof of any one of embodiments 44-52, wherein the alpha chain further comprises an alpha constant (Cα) region and/or the beta chain further comprises a beta constant (Cβ) region.
   54. The TCR or antigen-binding fragment thereof of embodiment 53, wherein the Cα region comprises the amino acid sequence of SEQ ID NO: 7, or an amino acid sequence having at least 80%, 85%, 86%, 87%, 88%, 89%, 90%, 91%, 92%, 93%, 94%, 95%, 96%, 97%, 98%, or 99% sequence identity to the amino acid sequence of SEQ ID NO: 7; and/or the Cβ region comprises the amino acid sequence of SEQ ID NO: 11, or an amino acid sequence having at least 80%, 85%, 86%, 87%, 88%, 89%, 90%, 91%, 92%, 93%, 94%, 95%, 96%, 97%, 98%, or 99% sequence identity to the amino acid sequence of SEQ ID NO: 11.
   55. The TCR or antigen-binding fragment thereof of embodiment 53, wherein the Cα region comprises the amino acid sequence of SEQ ID NO: 7, or an amino acid sequence having at least 80%, 85%, 86%, 87%, 88%, 89%, 90%, 91%, 92%, 93%, 94%, 95%, 96%, 97%, 98%, or 99% sequence identity to the amino acid sequence of SEQ ID NO: 7; and the Cβ region comprises the amino acid sequence of SEQ ID NO: 11, or an amino acid sequence having at least 80%, 85%, 86%, 87%, 88%, 89%, 90%, 91%, 92%, 93%, 94%, 95%, 96%, 97%, 98%, or 99% sequence identity to the amino acid sequence of SEQ ID NO: 11.
   56. The TCR or antigen-binding fragment thereof of embodiment 53, wherein the Cα region comprises the amino acid sequence of SEQ ID NO: 7; and/or the Cβ region comprises the amino acid sequence of SEQ ID NO: 11.
   57. The TCR or antigen-binding fragment thereof of any one of embodiments 44-56, wherein the alpha chain comprises the amino acid sequence of SEQ ID NO: 2 or an amino acid sequence having at least 80%, 85%, 90%, or 95% identity to SEQ ID NO: 2; and/or the beta chain comprises the amino acid sequence of SEQ ID NO: 4 or an amino acid sequence having at least 80%, 85%, 90%, or 95% identity to SEQ ID NO: 4.
   58. The TCR or antigen-binding fragment thereof of any one of embodiments 44-57, that is chimeric.
   59. The TCR or antigen-binding fragment thereof of any one of embodiments 44-48, 51-55, and 57, that is humanized.
   60. A nucleic acid molecule encoding the TCR or antigen-binding fragment thereof of any one of embodiments 44-59, or an alpha or beta chain thereof, or a Vα region or Vβ region thereof.
   61. The nucleic acid molecule of embodiment 60, that is isolated.
   62. The nucleic acid molecule of embodiment 60 or embodiment 61, wherein the nucleic acid molecule is codon-optimized.
   63. The nucleic acid molecule of embodiment 60 or embodiment 61, wherein the nucleic acid molecule is not codon-optimized.
   64. The nucleic acid molecule of any one of embodiments 60-63, wherein the nucleic acid molecule is DNA.
   65. The nucleic acid molecule of embodiment 64, wherein the DNA is cDNA.
   66. The nucleic acid molecule of any one of embodiments 60-63, wherein the nucleic acid molecule is RNA.
   67. The nucleic acid molecule of any one of embodiments 60-66, wherein the TCR or antigen-binding fragment thereof that is encoded is humanized or chimeric.
   68. The nucleic acid molecule of any one of embodiments 60-66, wherein the TCR or antigen-binding fragment thereof that is encoded is murine.
   69. The nucleic acid molecule of any one of embodiments 60-68, comprising the nucleic acid sequence of SEQ ID NO: 1, or a nucleic acid sequence having at least 80%, 85%, 90%, 91%, 92%, 93%, 94%, 95%, 96%, 97%, 98%, or 99% sequence identity to the nucleic acid sequence of SEQ ID NO: 1; and/or the nucleic acid sequence of SEQ ID NO: 3, or a nucleic acid sequence having at least 80%, 85%, 90%, 91%, 92%, 93%, 94%, 95%, 96%, 97%, 98%, or 99% sequence identity to the nucleic acid sequence of SEQ ID NO: 3.
   70. A vector comprising the nucleic acid molecule of any one of embodiments 60-69.
   71. A vector comprising a nucleic acid molecule encoding the TCR or antigen-binding fragment thereof of any one of embodiments 44-59, or an alpha and/or beta chain thereof, or a Vα region and/or Vβ region thereof.
   72. The vector of embodiment 70 or embodiment 71, wherein the vector is an expression vector.
   73. The vector of any one of embodiments 70-72, wherein the vector is a viral vector.
   74. The vector of embodiment 73, wherein the viral vector is selected from the group consisting of a retroviral vector, a gammaretroviral vector, a lentiviral vector, and an adeno-associated viral (AAV) vector.
   75. The vector of embodiment 74, wherein the AAV vector is a self-complementary AAV (scAAV) vector.
   76. The vector of any one of embodiments 70-72, wherein the vector is a non-viral vector.
   77. The vector of any one of embodiments 70-72, wherein the vector is a donor vector for genome editing.
   78. The vector of any one of embodiments 70-72, wherein the vector is a transposon vector.
   79. The vector of embodiment 78, wherein the transposon vector is a Sleeping Beauty transposon vector or a PiggyBac transposon vector.
   80. The vector of any one of embodiments 70-79, wherein the vector is suitable for gene editing or genomic engineering.
   81. An engineered cell comprising the nucleic acid molecule of any one of embodiments 60-69 or the vector of any one of embodiments 70-80.
   82. An engineered cell comprising the TCR or antigen-binding fragment thereof of any one of embodiments 44-59.
   83. The engineered cell of embodiment 82, wherein the TCR or antigen-binding fragment thereof is heterologous to the engineered cell.
   84. A method of producing an engineered cell, comprising introducing a nucleic acid molecule of any one of embodiments 60-69 or a vector of any one of embodiments 70-80, into a cell.
   85. A method of producing the engineered cell of any one of embodiments 1-43, comprising introducing a nucleic acid molecule of any one of embodiments 60-69 or a vector of any one of embodiments 70-80, into a cell.
   86. The method of embodiment 84 or embodiment 85, that is performed in vitro or ex vivo.
   87. The method of any one of embodiments 84-86, wherein the nucleic acid molecule is comprised within a vector.
   88. A method of producing a population of engineered cells, comprising introducing a nucleic acid molecule of any one of embodiments 60-69 or a vector of any one of embodiments 70-80, into a cell; and culturing the cell under conditions to produce a population of engineered cells.
   89. A method of producing a population of engineered cells, comprising culturing the engineered cell of any one of embodiments 1-43 under conditions to produce a population of engineered cells.
   90. A method of engineering a cell, comprising introducing a nucleic acid molecule of any one of embodiments 60-69 or a vector of any one of embodiments 70-80, into a cell.
   91. A method of engineering a cell, comprising:
introducing a nucleic acid molecule of any one of embodiments 60-69 or a vector of any one of embodiments 70-80, into a cell; and
editing and/or disrupting one or more genes endogenous to the cell.
   92. The method of any one of embodiments 84-88, 90, and 91, wherein the introducing is carried out by transfection, electroporation, or transduction.
   93. The method of any one of embodiments 84-88, 90, and 91, wherein the introducing is carried out by transfection.
   94. The method of embodiment 93, wherein the introducing by transfection comprises introducing a vector of any one of embodiments 70-80 into the cell.
   95. The method of any one of embodiments 84-88, 90, and 91, wherein the introducing is carried out by electroporation.
   96. The method of any one of embodiments 83-88, 90, and 91, wherein the introducing is carried out by transduction.
   97. The method of embodiment 96, wherein the introducing by transduction comprises introducing a vector of any one of embodiments 70-80 into the cell.
   98. The method of any one of embodiments 84-88 and 90-97, wherein the vector is a viral vector.
   99. The method of embodiment 98, wherein the viral vector is selected from the group consisting of a retroviral vector, a gammaretroviral vector, a lentiviral vector, and an adeno-associated viral (AAV) vector.
   100. The method of embodiment 99, wherein the AAV vector is a self-complementary AAV (scAAV) vector.
   101. The method of any one of embodiments 84-88 and 90-100, wherein the introducing is carried out using a genome editing technique.
   102. The method of any one of embodiments 84-88 and 90-100, the introducing is carried out using a genome editing technique; and/or the method further comprises a genome editing technique.
   103. The method of embodiment 101 or embodiment 102, wherein the genome editing technique results in editing and/or disrupting one or more genes endogenous to the cell; and/or introducing the nucleic acid molecule encoding the TCR or antigen-binding fragment thereof into a target site.
   104. The method of embodiment 103, wherein the target site is comprised within one or more genes endogenous to the cell.
   105. The method of embodiment 104, wherein the one or more genes endogenous to the cell comprises a T Cell Receptor Alpha Constant (TRAC) gene and/or a T Cell Receptor Beta Constant (TRBC) gene.
   106. The method of any one of embodiments 101-105, wherein the genome editing technique comprises CRISPR-Cas9 and comprises introducing a crRNA sequence targeting a human TRAC gene that comprises a nucleic acid sequence set forth in SEQ ID NO: 22, and/or comprises introducing a crRNA sequence targeting a human TRBC gene that comprises a nucleic acid sequence set forth in SEQ ID NO: 23.
   107. The method of any one of embodiments 84-106, further comprising introducing into the cell one or more agents capable of editing and/or disrupting one or more genes endogenous to the cell.
   108. The method of any one of embodiments 107, wherein the editing and/or disrupting is carried out by one or more agents capable of editing and/or disrupting the one or more genes endogenous to the cell.
   109. The method of embodiment 107 or embodiment 108, wherein the editing and/or disrupting reduces or eliminates expression of the one or more genes endogenous to the cell.
   110. The method of any one of embodiments 107-109, wherein the editing and/or disrupting eliminates expression of the one or more genes endogenous to the cell.
   111. The method of any one of embodiments 107-110, wherein the one or more genes endogenous to the cell each comprise a target site, and one or more of the one or more agents specifically bind to or recognizes the target site.
   112. The method of any one of embodiments 107-111, wherein the one or more genes endogenous to the cell comprises a T Cell Receptor Alpha Constant (TRAC) gene and/or a T Cell Receptor Beta Constant (TRBC) gene.
   113. The method of any one of embodiments 107-112, wherein the one or more agents comprises a nuclease.
   114. The method of embodiment 113, wherein the nuclease specifically binds to or recognizes the target site.
   115. The method of embodiment 113 or embodiment 114, wherein the nuclease is selected from the group consisting of a meganuclease, a zinc-finger nuclease, a transcription activator-like effector nuclease (TALEN), a megaTAL nuclease, and a clustered regularly interspaced short palindromic repeats (CRISPR)-associated (Cas) nuclease.
   116. The method of any one of embodiments 113-115, wherein the nuclease is a Cas nuclease.
   117. The method of embodiment 116, wherein the Cas nuclease is a Cas9, Cas12a, or Cas13 nuclease.
   118. The method of any one of embodiments 113-117, wherein the Cas nuclease is a Cas9 nuclease.
   119. The method of any one of embodiments 84-118, further comprising introducing into the cell one or more agents capable of inserting the nucleic acid molecule into the genome of the cell.
   120. The method of embodiment 119, wherein the one or more agents capable of inserting the nucleic acid molecule into the genome of the cell comprises a transposon or a transposon-based system.
   121. The method of embodiment 120, wherein the transposon comprises a Sleeping Beauty transposon or a PiggyBac transposon; or the transposon-based system comprises a Sleeping Beauty transposon-based system or a PiggyBac transposon-based system.
   122. A method of engineering a cell, comprising:
introducing, into the cell, one or more agents capable of editing and/or disrupting one or more endogenous genes in the cell, wherein each of the one or more endogenous genes comprises a first flanking sequence and a second flanking sequence; and
introducing, into the cell, one or more nucleic acid molecules, wherein each of the one or more nucleic acid molecules comprises: (i) a first homology arm and a second homology arm that are homologous to the first flanking sequence and the second flanking sequence of one of the one or more endogenous genes, and (ii) a nucleic acid sequence of interest that is located between the first homology arm and the second homology arm.
   123. A method of engineering a cell, comprising:
introducing, into the cell, one or more agents capable of inducing a DNA break in one or more endogenous genes in the cell, wherein each of the one or more endogenous genes comprises a first flanking sequence and a second flanking sequence; and
introducing, into the cell, one or more nucleic acid molecules, wherein each of the one or more nucleic acid molecules comprises: (i) a first homology arm and a second homology arm that are homologous to the first flanking sequence and the second flanking sequence of one of the one or more endogenous genes, and (ii) a nucleic acid sequence of interest that is located between the first homology arm and the second homology arm.
   124. The method of embodiment 122 or embodiment 123, wherein the nucleic acid sequence of interest encodes a T cell receptor (TCR) or antigen-binding fragment thereof, wherein the TCR or antigen-binding fragment thereof comprises an alpha chain comprising a variable alpha (Vα) region and a beta chain comprising a variable beta (Vβ) region, wherein the Vα region comprises a complementarity determining region 1 (CDR-1), CDR-2, and CDR-3 comprising the amino acid sequences of SEQ ID NOs: 12, 13, and 14, respectively; and the Vβ region comprises a CDR-1, CDR-2, and CDR-3 comprising the amino acid sequences of SEQ ID NOs: 15, 16, and 17, respectively.
   125. The method of any one of embodiments 122-124, wherein the nucleic acid sequence of interest comprises the nucleic acid molecule of any one of embodiments 60-69.
   126. The method of any one of embodiments 122-125, wherein the one or more endogenous genes comprises a T Cell Receptor Alpha Constant (TRAC) gene and/or a T Cell Receptor Beta Constant (TRBC) gene.
   127. The method of any one of embodiments 122 and 124-126, wherein the one or more agents capable of editing and/or disrupting one or more endogenous genes in the cell comprises a nuclease.
   128. The method of any one of embodiments 123-126, wherein the one or more agents capable of inducing a DNA break comprises a nuclease.
   129. The method of embodiment 127 or embodiment 128, wherein the nuclease is selected from the group consisting of a meganuclease, a zinc-finger nuclease, a transcription activator-like effector nuclease (TALEN), a megaTAL nuclease, and a clustered regularly interspaced short palindromic repeats (CRISPR)-associated (Cas) nuclease.
   130. The method of any one of embodiments 127-129, wherein the nuclease is a Cas nuclease.
   131. The method of embodiment 130, wherein the Cas nuclease is a Cas9, Cas12a, or Cas13 nuclease.
   132. The method of embodiment 130 or embodiment 131, wherein the Cas nuclease is a Cas9 nuclease.
   133. The method of any one of embodiments 122, 124-127, and 129-132, wherein the one or more agents capable of editing and/or disrupting one or more endogenous genes in the cell comprises a Cas nuclease and one or more single guide RNA (sgRNA).
   134. The method of any one of embodiments 123-126 and 128-132, wherein the one or more agents capable of inducing a DNA break comprises a Cas nuclease and one or more single guide RNA (sgRNA).
   135. The method of embodiment 133 or embodiment 134, wherein each of the one or more sgRNA specifically binds to, hybridizes with, or recognizes a target sequence in one of the one or more endogenous genes.
   136. The method of any one of embodiments 133-135, wherein the one or more sgRNA comprises an sgRNA that specifically binds to, hybridizes with, or recognizes a target sequence in an endogenous TRAC gene, and/or comprises an sgRNA that specifically binds to, hybridizes with, or recognizes a target sequence in an endogenous TRBC gene.
   137. The method of any one of embodiments 122-136, wherein the method results in reduced or eliminated expression of the one or more endogenous genes; and/or introduces expression of the TCR or antigen-binding fragment thereof in the cell.
   138. The method of any one of embodiments 122-137, wherein the method results in reduced or eliminated expression of the one or more endogenous genes and incorporation of a nucleic acid molecule encoding the TCR or antigen-binding fragment thereof into a target site contained within the one or more endogenous genes.
   139. The method of any one of embodiments 122-138, wherein the one or more nucleic acid molecules is comprised within a vector.
   140. The method of embodiment 139, wherein the vector is a donor vector.
   141. The method of embodiment 139, wherein the vector is an expression vector.
   142. The method of any one of embodiments 139-141, wherein the vector is a viral vector.
   143. The method of embodiment 142, wherein the viral vector is selected from the group consisting of a retroviral vector, a gammaretroviral vector, a lentiviral vector, and an adeno-associated viral (AAV) vector.
   144. The method of embodiment 143, wherein the AAV vector is a self-complementary AAV (scAAV) vector.
   145. The method of any one of embodiments 139-141, wherein the vector is a non-viral vector.
   146. The method of any one of embodiments 139-141, wherein the vector is a donor vector for genome editing.
   147. The method of any one of embodiments 139-141, wherein the vector is a transposon vector.
   148. The method of embodiment 147, wherein the transposon vector is a Sleeping Beauty transposon vector or a PiggyBac transposon vector.
   149. The vector of any one of embodiments 139-148, wherein the vector is suitable for gene editing or genomic engineering.
   150. The method of any one of embodiments 122-149, wherein the introducing of one or more of: (i) the one or more agents capable of editing and/or disrupting one or more endogenous genes in the cell, (ii) one or more agents capable of inducing a DNA break in one or more endogenous genes in the cell, and/or (iii) one or more nucleic acid molecules, is carried out by transfection, electroporation, or transduction, or any combination thereof.
   151. The method of embodiment 150, wherein the introducing of one or more of: (i) the one or more agents capable of editing and/or disrupting one or more endogenous genes in the cell, (ii) one or more agents capable of inducing a DNA break in one or more endogenous genes in the cell, and/or (iii) one or more nucleic acid molecules, is carried out by transfection.
   152. The method of embodiment 151, wherein the introducing of the one or more nucleic acid molecules by transfection comprises introducing a vector of any one of embodiments 70-80 into the cell.
   153. The method of any one of embodiments 122-152, wherein the introducing one or more of: (i) the one or more agents capable of editing and/or disrupting one or more endogenous genes in the cell, (ii) one or more agents capable of inducing a DNA break in one or more endogenous genes in the cell, and/or (iii) one or more nucleic acid molecules, is carried out by electroporation.
   154. The method of any one of embodiments 122-153, wherein the introducing one or more of: (i) the one or more agents capable of editing and/or disrupting one or more endogenous genes in the cell, (ii) one or more agents capable of inducing a DNA break in one or more endogenous genes in the cell, and/or (iii) one or more nucleic acid molecules, is carried out by transduction.
   155. The method of embodiment 154, wherein the introducing the one or more nucleic acid molecules by transduction comprises introducing a vector of any one of embodiments 70-80 into the cell.
   156. The method of any one of embodiments 122-155, wherein the introducing the one or more nucleic acid molecules occurs by a transfection method.
   157. The method of embodiment 156, wherein the transfection method is electroporation.
   158. The method of any one of embodiments 84-157, wherein the cell is from a subject having a disease or condition associated with amyloid beta.
   159. The method of embodiment 158, wherein the disease or condition associated with amyloid beta is a disease or condition associated with human amyloid beta.
   160. The method of embodiment 158, wherein the disease or condition associated with amyloid beta is Alzheimer's Disease.
   161. The method of any one of embodiments 84-157, wherein the cell is from a subject having Alzheimer's Disease.
   162. The method of any one of embodiments 84-157, wherein the cell is from a donor subject.
   163. The method of embodiment 162, wherein the donor subject does not have a disease or condition associated with amyloid beta or has not been diagnosed as having a disease or condition associated with amyloid beta.
   164. The method of embodiment 162 or embodiment 163, wherein the donor subject does not have Alzheimer's Disease or has not been diagnosed as having Alzheimer's Disease.
   165. The method of any one of embodiments 84-164, wherein the cell is a T cell, a B cell, or a Natural Killer (NK) cell.
   166. The method of embodiment 165, wherein the engineered cell is a T cell.
   167. The method of embodiment 166, wherein the T cell is a regulatory T cell or an effector T cell.
   168. The method of embodiment 166 or embodiment 167, wherein the T cell is a regulatory T cell.
   169. The method of embodiment 166 or embodiment 167, wherein the T cell is a CD4+ and/or CD8+ T cell.
   170. The method of any one of embodiments 166-168, wherein the T cell is a CD4+/CD25+/FOXP3+ T cell.
   171. An engineered cell produced by the method of any one of embodiments 84-170.
   172. The engineered cell of any one of embodiments 1-43 and 171, wherein the engineered cell is a regulatory T cell.
   173. A conjugate, comprising the TCR or antigen-binding fragment thereof of any one of embodiments 44-59, and a heterologous moiety.
   174. The conjugate of embodiment 173, wherein the heterologous moiety is a detectable label.
   175. The conjugate of embodiment 174, wherein the detectable label is a fluorescent label.
   176. The conjugate of embodiment 174, wherein the detectable label is a radioisotope, a fluorescent label, or an enzyme-substrate.
   177. A composition, comprising the engineered cells of any one of embodiments 1-43 and 171, or the TCR or antigen-binding fragment thereof of any one of embodiments 44-59, or the conjugate of any one of embodiments 173-176.
   178. The composition of embodiment 177, further comprising a pharmaceutically acceptable excipient.
   179. A method of treatment of a disease or condition associated with amyloid beta, comprising administering the engineered cell of any one of embodiments 1-43 and 171, or the composition of embodiment 177 or embodiment 178, to a subject having a disease or condition associated with amyloid beta.
   180. The method of embodiment 179, wherein the disease or condition associated with amyloid beta is Alzheimer's Disease.
   181. A method of treatment of Alzheimer's Disease, comprising administering the engineered cell of any one of embodiments 1-43 and 171, or the composition of embodiment 177 or embodiment 178, to a subject having Alzheimer's Disease.
   182. A method of treatment of a disease or condition associated with amyloid beta, comprising administering the composition of embodiment 177 or embodiment 178 to a subject having a disease or condition associated with amyloid beta.
   183. The method of embodiment 182, wherein the disease or condition associated with amyloid beta is Alzheimer's Disease.
   184. A method of treatment of a disease or condition associated with amyloid beta, comprising administering the composition of embodiment 177 or embodiment 178 to a subject having Alzheimer's Disease.
   185. A method of treating a disease or condition associated with amyloid beta, comprising:
a) engineering a cell by the method of any one of embodiments 84-170;
b) culturing the engineered cell under conditions to produce a population of engineered cells; and
c) administering a therapeutically effective amount of the population of engineered cells to a subject having a disease or condition associated with amyloid beta.
   186. A method of treating Alzheimer's Disease, comprising:
a) engineering a cell by the method of any one of embodiments 84-170;
b) culturing the engineered cell under conditions to produce a population of engineered cells; and
c) administering a therapeutically effective amount of the population of engineered cells to a subject having Alzheimer's Disease.
   187. The method of any one of embodiments 179-186, wherein the subject is a human.
   188. The method of any one of embodiments 179-187, wherein the engineered cells are autologous to the subject.
   189. The method of any one of embodiments 179-187, wherein the engineered cells are allogenic to the subject.
   190. The method of any one of embodiments 179-189, wherein the engineered cells are T cells, B cells, or Natural Killer (NK) cells.
   191. The method of embodiment 190, wherein the engineered cells are T cells.
   192. The method of embodiment 191, wherein the T cells are regulatory T cells or effector T cells.
   193. The method of embodiment 191, wherein the T cells are regulatory T cells.
   194. The method of embodiment 192 or embodiment 193, wherein the T cells are CD4+ and/or CD8+ T cells.
   195. The method of embodiment 192 or embodiment 193, wherein the T cells are CD4+/CD25+/FOXP3+ T cells.
   196. A composition of embodiment 177 or embodiment 178 for use in treating a disease or condition associated with amyloid beta in a subject.
   197. Use of a composition of embodiment 177 or embodiment 178 for the manufacture of a medicament for treating a disease or condition associated with amyloid beta in a subject.
   198. The composition for use of embodiment 196 or the use of embodiment 197, wherein the subject is a human.
   199. The composition for use of embodiment 196 or embodiment 198, or the use of embodiment 197 or embodiment 198, wherein the disease or condition associated with amyloid beta is Alzheimer's Disease.
   200. A method of diagnosing a disease or condition associated with amyloid beta, comprising administering the TCR or antigen-binding fragment thereof of any one of embodiments 44-59, or the conjugate of any one of embodiments 173-176, or the composition of embodiment 177 or embodiment 178, to a subject having or suspected of having a disease or condition associated with amyloid beta.
   201. The method of embodiment 200, further comprising detecting the level or absence of binding between the TCR or antigen-binding fragment thereof or the conjugate with amyloid beta.
   202. The method of embodiment 201, further comprising comparing the level or absence of binding to the level or absence of binding between the TCR or antigen-binding fragment thereof or the conjugate with amyloid beta as detected as one or more preceding time points.
   203. A method of monitoring the progression of a disease or condition associated with amyloid beta, comprising administering the TCR or antigen-binding fragment thereof of any one of embodiments 44-59 or the conjugate of any one of embodiments 173-176, or the composition of embodiment 177 or embodiment 178, to a subject having a disease or condition associated with amyloid beta.
   204. The method of embodiment 203, further comprising detecting the level or absence of binding between the TCR or antigen-binding fragment thereof or the conjugate with amyloid beta.
   205. The method of embodiment 204, further comprising comparing the level or absence of binding to the level or absence of binding between the TCR or antigen-binding fragment thereof or the conjugate with amyloid beta as detected as one or more preceding time points.
   206. A method of diagnosing Alzheimer's Disease, comprising administering the TCR or antigen-binding fragment thereof of any one of embodiments 44-59, or the conjugate of any one of embodiments 173-176, or the composition of embodiment 177 or embodiment 178, to a subject having or suspected of having Alzheimer's Disease.
   207. A method of monitoring the progression of Alzheimer's Disease, comprising administering the TCR or antigen-binding fragment thereof of any one of embodiments 44-59, or the conjugate of any one of embodiments 173-176, or the composition of embodiment 177 or embodiment 178, to a subject having Alzheimer's Disease.
   208. A composition of embodiment 177 or embodiment 178 for use in treating a disease or condition associated with amyloid beta in a subject.
   209. Use of a composition of embodiment 177 or embodiment 178 for the manufacture of a medicament for treating a disease or condition associated with amyloid beta in a subject.
   210. A composition of embodiment 177 or embodiment 178 for use in treating Alzheimer's Disease in a subject.
   211. Use of a composition of embodiment 177 or embodiment 178 for the manufacture of a medicament for treating Alzheimer's Disease in a subject.
   212. The composition for use of embodiment 208 or embodiment 210, or the use of embodiment 209 or embodiment 211, wherein the subject is a human.

### XI. EXAMPLES

The following examples are included for illustrative purposes only and are not intended to limit the scope of the invention.

### Example 1 Generation and Characterization of Aβ-Th1 and Aβ-Th17 Cell Phenotypes

This Example describes a method for the generation of stable Aβ-Th1 and Aβ-Th17 cell, as well as their phenotypic characterization.

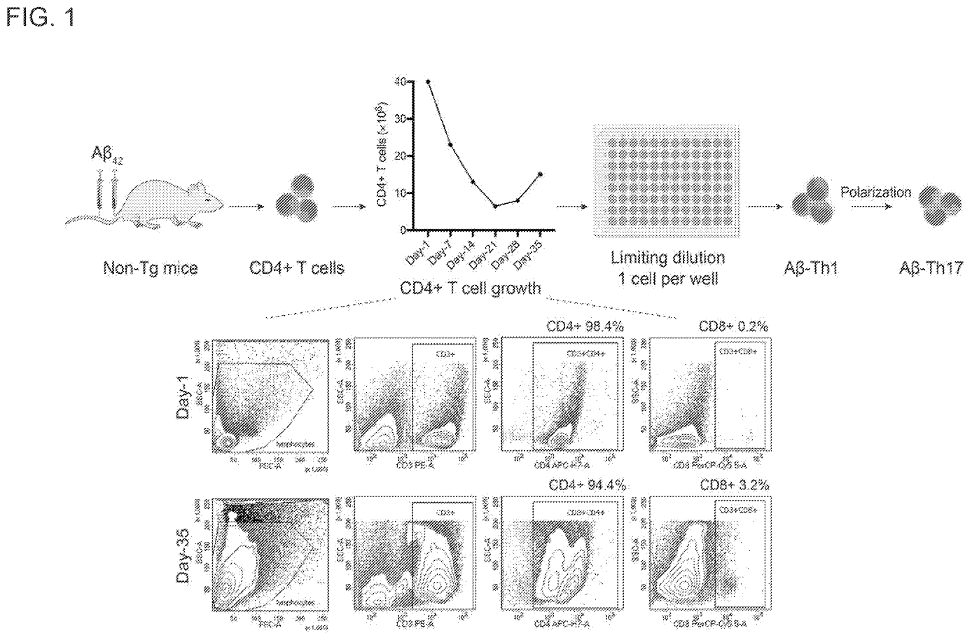

Previous works developed antigen-specific T cells from Aβ₁₋₄₂ immunization of non-Tg mice. However, the cells were polarized for a short period prior to adoptive transfers with uncertain phenotypes (Browne et al., 2013, J Immunol, 190, 2241-2251; Mittal et al., 2019, iScience, 16, 298-311). Herein, stable Aβ-Teff clones were developed and were cultured for more than six months retaining antigen specificity and phenotype. Briefly, six-months old, two female C57BL6 mice were immunized by subcutaneous injection with human Aβ₁₋₄₂ (50 μg) (Catalog no. 03-112, Life Technologies) emulsified in Freund's complete adjuvant (Sigma Aldrich) containing Mycobacterium tuberculosis (1 mg/ml). The mice were boosted after two weeks with Aβ₁₋₄₂ (50 μg) in Freund's incomplete adjuvant (Sigma Aldrich). One week after the second immunization the mice were sacrificed and spleen and lymph nodes (axial, brachial, cervical, and inguinal) harvested then mashed through a 70 μm cell strainer to prepare single cell suspensions. The red blood cells were lysed from the splenocytes using an Ammonium-Chloride-Potassium (ACK) lysis buffer (Catalog no. A10492, Thermo Fisher Scientific). CD4+ T cells were isolated from single cell suspensions using an EasySep™ mouse CD4+ T cell isolation kit (Catalog No. 19852, Stemcell Technologies). The recovered 98% pure CD4+ T cells were cultured in vitro in the presence of 2.5×10⁷ feeder cells (splenocytes isolated from female C57BL6 mice) that were irradiated with 3200 Gy using a RS 2000 X-Ray irradiator and stimulated with monomeric Aβ₁₋₄₂ (25 μg/ml) prepared as described before (Kiyota et al., 2013, Neurobiol Aging, 34, 1060-1068) (Catalog no. RP10017, GenScript). Cells were propagated using RPMI-1640 media supplemented with 10% fetal bovine serum (FBS), 2 mM L-glutamine, 25 mM HEPES, 1 mM sodium pyruvate, 1× nonessential amino acids, 55 nM 2-mercaptoethanol, 100 U/ml penicillin, and 100 μg streptomycin. Fresh culture media was provided every week and the emergence of viable CD4+ T cells counted before each passage to assess cell growth. All CD4+ T cells non-reactive to Aβ were eliminated as evidence by declined viable cell numbers at day 21. Following this increase in CD4+ T cell counts were observed beginning at day 28. At this point 25 U/ml interleukin-2 (IL-2) was added to the culture and leading to increases in numbers of CD4+ T cells to day 35 (FIG. 1). To recover monoclonal CD4+ T cell clones, cells were cultured in a limiting dilution assay at a density of 1 cell/well in flat-bottom 96 well plate in presence of fresh feeder cells and Aβ₁₋₄₂. The most rapid growing Aβ reactive clone was identified and cultured with IL-2 and hereafter assigned as Aβ-Th1 cells. The Aβ-Th1 cells includes a TCR having the amino acid (aa) and nucleic acid (nucleotide; nt) sequences as shown in Table E1.

TABLE E1
Aβ-Th1 Sequences (amino acid = aa; nucleotide = nt)
Alpha chain

| | Variable | | | | | | |
|---|---|---|---|---|---|---|---|
| Alpha | (Vα) | | | | Junction | Constant | Alpha |
| chain | region | CDR-1 | CDR-2 | CDR-3 | region | region | chain |
| (aa) | (aa) | (aa) | (aa) | (aa) | (aa) | (aa) | (nt) |
| SEQ ID | SEQ ID | SEQ ID | SEQ ID | SEQ ID | SEQ ID | SEQ ID | SEQ ID |
| NO: 2 | NO: 5 | NO: 12 | NO: 13 | NO: 14 | NO: 6 | NO: 7 | NO: 1 |

Beta chain

| | Variable | | | | Diversity | Junction | Constant | Beta |
|---|---|---|---|---|---|---|---|---|
| Beta | (Vβ) | | | | region | region | region | chain |
| chain | region | CDR-1 | CDR-2 | CDR-3 | (aa) | (aa) | (aa) | (nt) |
| SEQ ID | SEQ ID | SEQ ID | SEQ ID | SEQ ID | SEQ ID | SEQ ID | SEQ ID | SEQ ID |
| NO: 4 | NO: 8 | NO: 15 | NO: 16 | NO: 17 | NO: 9 | NO: 10 | NO: 11 | NO: 3 |

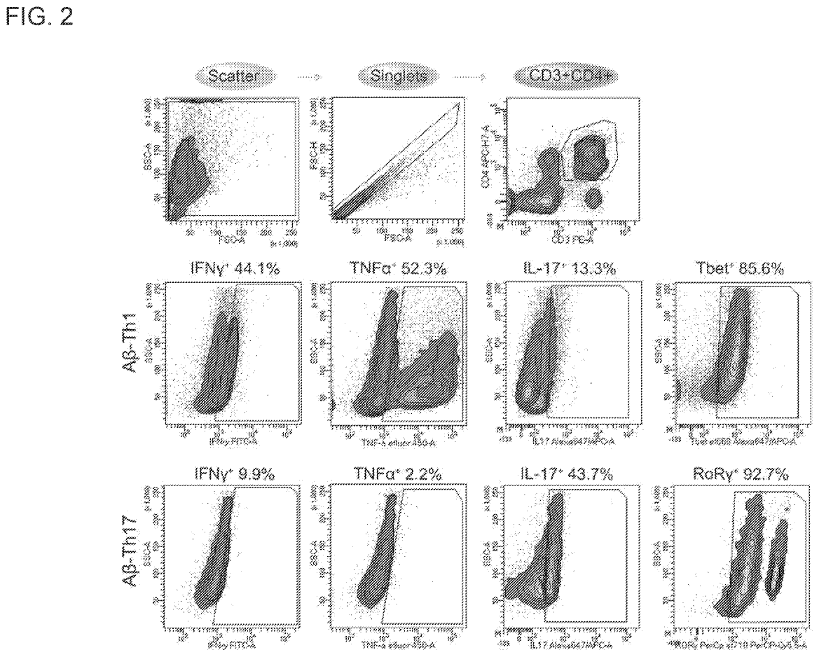

The Aβ-Th1 cells were polarized into the Th17 cells hereafter assigned as Aβ-Th17 using 3 ng/ml TGFβ, 25 ng/ml IL-6, 5 ng/ml IL-1B, 20 ng/ml IL-23 and 3 μg/ml of antibodies to IL4, IFNγ, and IL-2 and cultured in the presence of fresh feeder cells with Aβ₁₋₄₂. Intracellular cytokines of the cells were assessed by flow cytometry. Briefly, 10⁶ Aβ-Th1 or Th17 cells were stimulated with complete RPMI-1640 media containing 20 ng/ml phorbol-12-myristate-13-acetate (PMA), 1 μM ionomycin (Sigma Aldrich) and Brefeldin A (Catalog no. 4506521, eBioscience) for 12 hr then stained for intracellular cytokines. After incubation time, cells were first stained extracellularly with anti-mouse CD3e-PE (Catalog no. 12003181, eBioscience), anti-mouse CD4-APC-H7 (Catalog no. 560181, BD Pharmingen), and anti-mouse CD8a-Pe-Cyanine 5.5 (Catalog no. 35008180, eBioscience) antibodies for 30 min at room temperature. For intracellular staining, cells were fixed and permeabilized using transcription factor staining buffer kit (Catalog no. 552300, eBioscience) for 45 min at 4° C., followed by incubation with anti-mouse IFNγ-FITC (Catalog no. 11731182, eBioscience), TNF-α-eFluor 450 (Catalog no. 48732182, eBioscience), IL17a-Alexa Fluor 647 (Catalog no. 506912, BioLegend), Tbet-Efluor 660 (Catalog no. 50582582, eBioscience) and RoRγ-PerCp Efluor 710 antibodies (Catalog no. 46698182, eBioscience) for 30 min at 4° C. An isotype control and fluorescence-minus-one (FMO) control for each antibody were used during flow cytometry staining for the accurate gating of different cell subsets. All the samples were analyzed using BD LSR II flow cytometer and FACSDiva software (BD Biosciences) at the University of Nebraska Medical Center flow cytometry research facility. The flow cytometry of intracellular cytokines revealed that activated Aβ-Th1 cells expressed pro-inflammatory cytokines IFNγ (44.1%) and tumor necrosis factor alpha (TNF-α) (52.3%) and expressed nuclear transcription factor T-box expressed in T cells (T-bet) (85.6%), while Aβ-Th17 cells selectively secreted interleukin 17 (IL-17) (43.7%) and expressed transcription factor RAR-related orphan receptor gamma (RORγ) (92.7%) (FIG. 2).

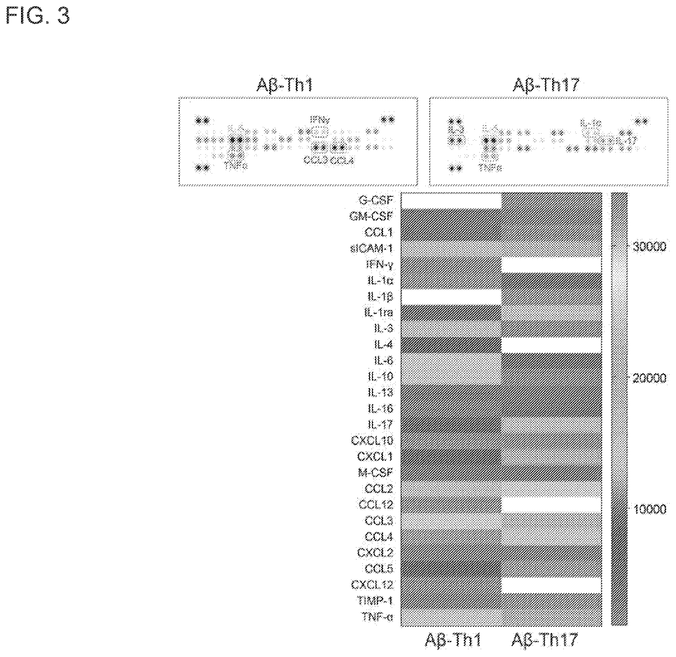

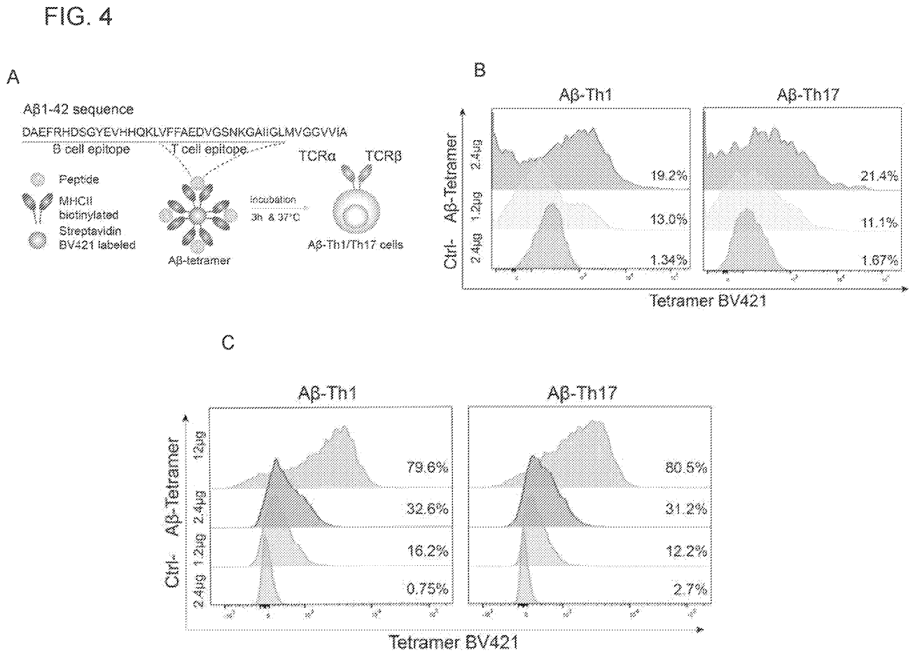

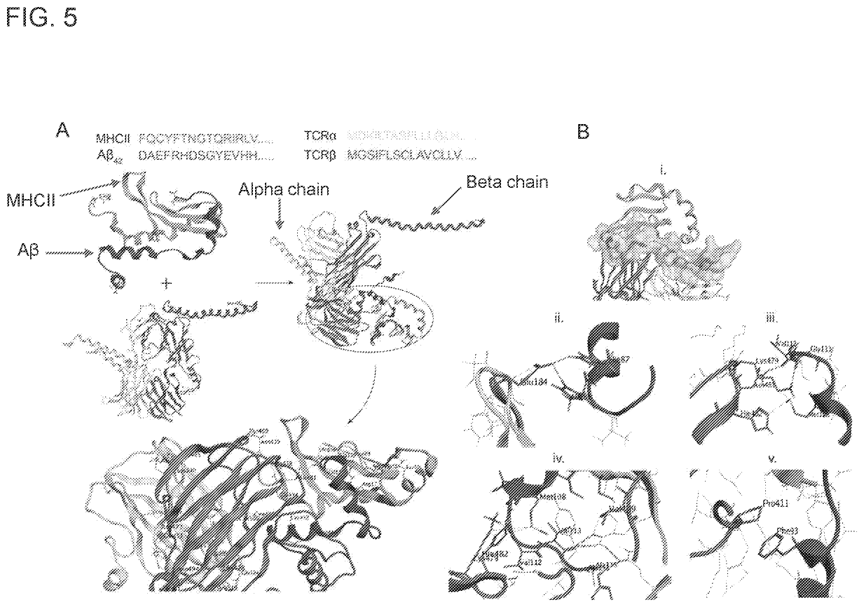

To confirm the cellular phenotype of Aβ-Teffs, extracellular cytokine release was assessed by immunostaining. For analysis of extracellular cytokine release, Aβ-Th1 and Aβ-Th17 cells were stimulated with 20 ng/ml PMA and 1 μM ionomycin for 12 hr and after incubation, cell supernatants were analyzed using proteome profiler mouse cytokine array kit (Catalog no. ARY006, R&D Systems) according to the manufacturer's instructions. The immunoblot staining for 42 target proteins showed that Aβ-Th1 cells selectively secreted IFNγ while Aβ-Th17 cells secreted higher IL17 (FIG. 3). Surprisingly both Aβ-Th1 and Aβ-Th17 cells secreted TNF-α after activation, higher expression was observed in Aβ-Th1 cells. To note, pure CD3+CD4+ T cells were gated to quantify the intracellular cytokine and transcription factor expression while in contrast few endogenous feeder cells were present during extracellular immunoblot staining that might have affected the cellular cytokine signature during short activation period. Aβ-Th1 and Th17 cells also upregulated chemokines CCL3 (MIP-1a) and CCL4 (MIP-1B), higher expression was observed with Aβ-Th1 cells. CCL3 and CCL4 have been implicated in different inflammatory conditions and are produced in substantial quantities by the Th1 type lymphocytes (Schrum et al., 1996, J Immunol, 157, 3598-3604), further supporting cellular phenotype of Aβ-Teffs. T cells' unique receptor (T cell receptor, TCR) recognize cognate antigen when presented by the APCs via MCH molecules (J. et al., 2020, Mol Neurodegener, In press; Alberts et al., 2002, Molecular Biology of the Cell. 4th edition). Therefore, to determine the antigen specificity of the stably maintained monoclonal Teff clones, Aβ-Th1 and Th17 cells were incubated with fluorescently labelled MHCII-IA^{b}-KLVFFAEDVGSNKGA (T cell epitope) tetramer (FIG. 4A). Briefly, from the Aβ₄₂ sequence, the 1 to 15 amino acid region (DAEFRHDSGYEVHHQ) comprises B cell epitope while the 15 to 30 amino acid region (KLVFFAEDVGSNKGA) comprises T cell epitope in the mice, though in human it may be up to 15 to 42 amino acids region (Kiyota et al., 2013, Neurobiol Aging, 34, 1060-1068). To elucidate the ability of Aβ-Th1 and Aβ-Th17 cells to recognize the T cell epitope site, KLVFFAEDVGSNKGA peptide bound to MHCII H2-b haplotype (feeder cell matched) alloantigen IA^{b} tetramers (MHCII-IA^{b}-KLVFFAEDVGSNKGA) conjugated to fluorophore BV420 were prepared by the NIH tetramer core facility at Emory University. 3×10⁵ Aβ-Th1 or Aβ-Th17 cells were incubated with different concentrations of MHCII-IA^{b}-KLVFFAEDVGSNKGA tetramer (1.2 μg, 2.4 μg and 12 μg) for 3 hr at 37° C. After incubation, the T cell-MHCII-Aβ tetramer complex was stained with anti-mouse CD3e-PE and CD4-APC-H7 antibodies for 30 min at room temperature, followed by live-dead staining with propidium iodide (0.5 μg/ml) for 5 min at room temperature. The T cell-tetramer complex was analyzed using BD LSR II flow cytometer and FACSDiva Software (BD Bioscience) at the University of Nebraska Medical Center flow cytometry research facility. The three-month Aβ-Th1 and Th17 clones showed binding with MHC-peptide tetramer in a dose-dependent manner, confirming their Aβ specificity (FIG. 4B). However, prolonged maintenance of stable Teff clones potentiated their cognate antigen recognition seen through improved dose-dependent Aβ-Teff-MHC-peptide binding in FIG. 4C compared to the 3 months old clones, suggesting improved antigen specificity of Aβ-Teffs. From Aβ-Th1 cells, antigen recognizing variable regions of T cell receptor (TCR) alpha (α) and beta (β) chains were identified using molecular cloning (FIG. 5A). Molecular modeling of full length TCRα/β complex with Aβ₄₂-MHCII (H-2b) (pMHCII) complex was performed to determine exact amino acid interactions between two complexes. Regarding the pMHC complex, the MHCII molecule chain and the peptide chain are indicated accordingly. Regarding the two chains of TCR, the TCR-α chain and the TCR-β are indicated accordingly. The complete model for the TCR-pMHC complex and enlarged focused region is showing the interaction between the pMHC with the TCR and the interacting residues. Further, peptide surface at the interface of MHC and TCR is shown (FIG. 5B). The TCR-pMHC interactions are as follows: (ii) pMHC residues Gly111 (O) and Val112(O) interacts with Lys479 and Asn481 sidechains of TCR-β respectively to form hydrogen bond interactions; (iii) The ring nitrogen (NE2) of pMHC His86 and His87 interacts with the TCR-α Glu184(OE1); (iv) Lys479 (NZ) forms weaker Van der Waals interactions with Met108(O, SD); (v) A π-π interaction is observed between peptides Phe93 and the Pro411 of TCR-β Chain due to stacking of aromatic rings (FIG. 5B).

### Example 2 Assessment of Aβ-Teffs in Inflammation and Immunosuppression

### A. Aβ-Teffs Lead to Memory Impairments

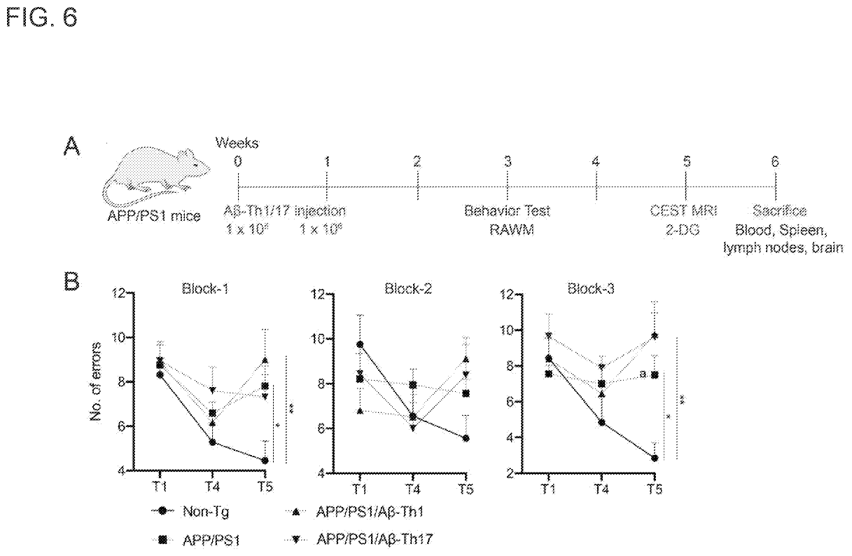

After confirmation of the stable cellular phenotype and antigen specificity, 2×10⁶ Aβ-Th1 or Aβ-Th17 cells were adoptively transferred into the APP/PS1 mice. Transgenic mice overexpressing human APP695 with the Swedish mutation (Tg2576) were obtained from Drs. G. Carlson and K. Hsiao-Ashe through the Mayo Medical Venture (Hsiao et al., 1996, Science, 274, 99-102). PS1 mice overexpressing human PS1 with M146L mutation were provided by Dr. K. Duff through the University of South Florida (Duff et al., 1996, Nature, 383, 710-713). Both mice were maintained in the B6; 129 hybrid background. Male Tg2576 mice were crossbred with female PS1 mice to generate APP/PS1 double-transgenic mice and non-transgenic (non-Tg) B6; 129 mice were developed in parallel as described previously (Kiyota et al., 2018, J Neuroimmunol, 319, 80-92; Kiyota et al., 2018, J Neuroinflammation, 15, 137). 4-5 months old female APP/PS1 mice and age matched non-Tg were used for the in vivo experiments. Mice were randomly divided into the different experimental groups. Either 1×10⁶ Aβ-Th1 or Aβ-Th17 cells in 100 μl PBS were intravenously injected using a 28-gauge needle affixed to a sterile tuberculin syringe into the APP/PS1 mice twice at one-week interval. Due to high Aβ-specificity of developed monoclonal CD4+ Teffs, such low cell count was adoptively transferred to study in vivo effects. Untreated and age matched non-Tg mice served as controls. In each experimental group, six mice were included. All the animal experiments were approved by the institutional Animal Care and Use Committee of University of Nebraska Medical Center. These experiments were performed in order to study the in vivo effects of the propagated clones (FIG. 6A). Two weeks after second adoptive cell transfer, mice were employed to the radial arm water maze (RAWM) test in a blinded fashion to assess memory impairment as previously described (Kiyota et al., 2018, J Neuroimmunol, 319, 80-92; Kiyota et al., 2013, Neurobiol Aging, 34, 1060-1068). Briefly, mice from masked cages were introduced into the circular water filled tank (diameter-110 cm and height-91 cm, San Diego Instruments) with triangular inserts that produce six swim paths radiating from the center. Special cues are fixed on the tank wall to guide mouse orientation. At the end of any one arm, a circular Plexiglas platform (diameter-10 cm) is placed submerged 1 cm beneath the water level to hide from the mice. The platform was placed in the same arm for four consecutive acquisition trials (T1-T4), and retention trial (T5), but in a different arm on different experimental day. For T1-T4, the mouse started the task from a randomly chosen arm without a platform. After four trials, the mouse was returned to its cage for 30 min and reintroduced into the T4 arm for the delayed retention trial (T5). Each trial lasted 1 min, and an error was scored when mouse entered the wrong arm; entered the arm with the platform, but did not climb on it; or did not make a choice for 20 sec. The trial ended when the mouse climbed and stay on the platform for at least 10 sec. The mouse allowed to rest on the platform for 20 sec between trials. If the mouse did not climb the platform, after 60 sec it was gently guided to the submerged platform. The T1, T4 and T5 trial errors over 9-days test were divided into three blocks, and the errors in each block were averaged for statistical analysis. As shown in FIG. 6B, APP/PS1 mice showed signs of memory impairment evidenced by significant increase in the number of errors in late retention trial T5 (p<0.05, block 1 and 3) compared to the non-Tg mice. For statistical analysis, all data were normally distributed and presented as mean values±standard errors of the mean (SEM). Comparisons of means between groups were analyzed by one-way ANOVA or two-way repeated measures ANOVA followed by Newman-Keuls post-hoc test using GraphPad Prizm software version 4.0. A value of p≤0.05 was regarded as a significant difference. Results were consistent with prior studies where cognitive impairment in the five months old APP/PS1 mice was demonstrated (Kiyota et al., 2018, J Neuroimmunol, 319, 80-92). Previously, the short-term cultured Aβ-Th1 cells but not Th17 cells showed cognitive impairments (Browne et al., 2013, J Immunol, 190, 2241-2251). In contrast, here higher number of cognitive errors were observed in APP/PS1 mice that received either Aβ-Th1 or Aβ-Th17 cells compared to non-Tg mice (FIG. 6B, Aβ-Th1 p<0.001 in block 1 and 3 while and Aβ-Th17 p<0.01 in block 3). Additionally, APP/PS1/Aβ-Th1/17 mice showed 30% higher number of experimental errors compared to the untreated APP/PS1 mice although the error increment was not significantly different. Overall, these results support the notion that Aβ-Th1 and Aβ-Th17 cells accelerate memory impairment in APP/PS1 mice that is linked to a pro-inflammatory phenotype under an amyloid enriched environment.

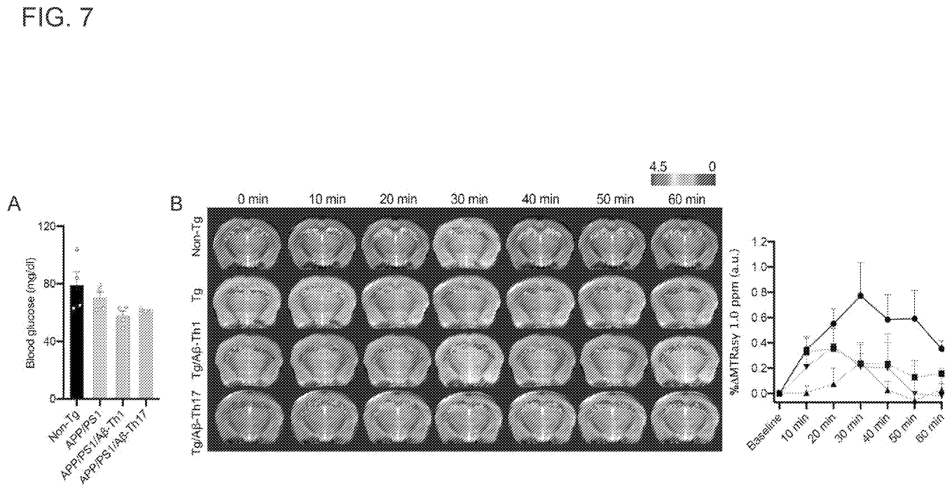

Brain glucose uptake and its subsequent metabolism is a biomarker for the memory impairment which was further used to confirm the effects of Aβ-Teffs on the cognitive behavior of APP/PS1 mice (Mosconi et al., 2013, J Alzheimers Dis, 35, 509-524; Niccoli et al., 2016, Curr Biol, 26, 2291-2300). ¹⁸F radiolabeled fluorodeoxyglucose (¹⁸F-FDG) positron emission tomography (PET) imaging is commonly used for the diagnosis of dementia type in the AD patients and in laboratory animal models (Chetelat et al., 2020, Lancet Neurol, 19, 951-962; Gordon et al., 2018, Lancet Neurol, 17, 241-250; Nordberg et al., 2010, Nat Rev Neurol, 6, 78-87; Bouter et al., 2019, Front Med (Lausanne), 6, 71). However, in this study, a non-radiolabeled 2-deoxy glucose (2DG) chemical exchange saturation transfer (CEST)-MRI was adopted. 2DG quickly enters the brain via the same transporters for the D-glucose, where it is metabolized into the 2DG-6-phosphate (2DG6P) but minimally metabolized. Due to low blood brain barrier (BBB) permeability, 2DG6P trapped inside the brain which can be detected by the CEST-MRI approach as described earlier (Nasrallah et al., 2013, J Cereb Blood Flow Metab, 33, 1270-1278; Meibach et al., 1980, Brain Res, 195, 167-176). Briefly, mice were fasted for 24 hr and fasting blood glucose concentrations were measured prior to the experiment. The mice were anesthetized with isoflurane in the mixture of oxygen. The peritoneal cavity was cannulated for the injection of 2DG) and mice were fixed on a 1H magnetic resonance imaging (MRI)-compatible cradle using a bite bar. MRI was performed on a 7 Tesla scanner (Bruker PharmaScan, Billerica, MA) with a Bruker-built quadrature moue brain RF coil. Respiration and body temperature were monitored during scanning. A baseline glucose CEST (glucoCEST) data were acquired followed by 2DG (1 gm/kg in PBS) injection via intraperitoneal catheter into the mice to monitor glucose signal in the brain over the time. GlucoCEST data were acquired using a RARE sequence (TR/TE=1600/16 ms, RARE factor=8) with a continuous RF for saturation with the power=3 μT, duration=1 s, saturation frequencies=−1600 to 1600 Hz in steps of 80 Hz. A second CEST data with saturation RF power=0.5 μT, and frequencies=−300 to +300 Hz were acquired for B0 inhomogeneity correction using WASSR (PMID: 19358232). The glucoCEST scan time is ˜10 mins, and repeated at 10, 20, 30, 40, 50, and 60 mins after 2DG injection. Asymmetric magnetization transfer ratio (MTRasym) was calculated from the Z-spectrum that was built based on CEST data. The glucoCEST signal was calculated as the integral of the MTRasym within 1.00±0.25 p.p.m. APP/PS1 mice demonstrated low GlucoCEST signal in the hippocampus, a region that primarily drive the cognitive functions, over different timepoints compared to the non-Tg mice as shown in the representative histographs (FIG. 7A-B). The area under curve (AUC) for APP/PS1 mice was quantified 28% low compared to the non-Tg mice group. However, AUC significantly reduced in the APP/PS1/Aβ-Th1 mice (p<0.01, 69%) compared to the non-Tg mice while APP/PS1/Aβ-Th17 mice showed 46% less AUC compared to the non-Tg mice (FIG. 7A-B) suggesting faster cognitive deficits in the APP/PS1 mice after Aβ-Th1 and Th17 cell adoptive transfer, further confirming the behavioral experiment results. Therefore, Aβ-Teffs have the ability to speed disease progression in the AD experimental mice by advancing memory impairments.

### B. Aβ-Teffs Promote Systemic Inflammation

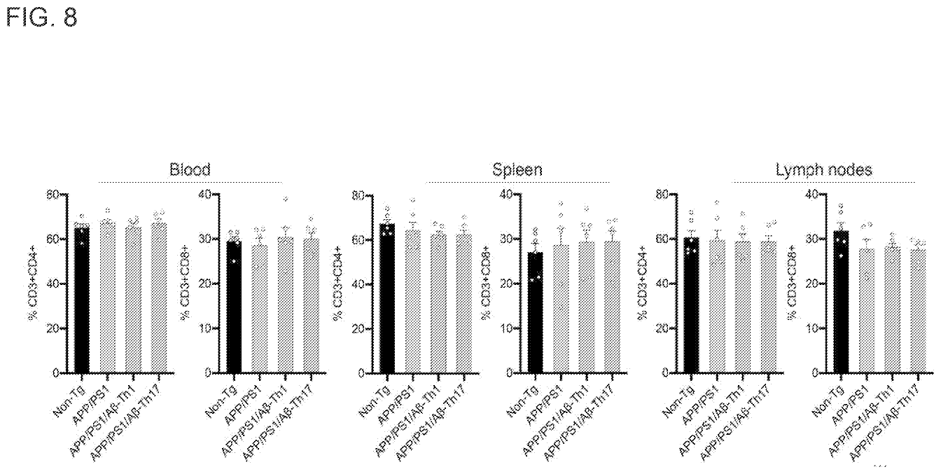

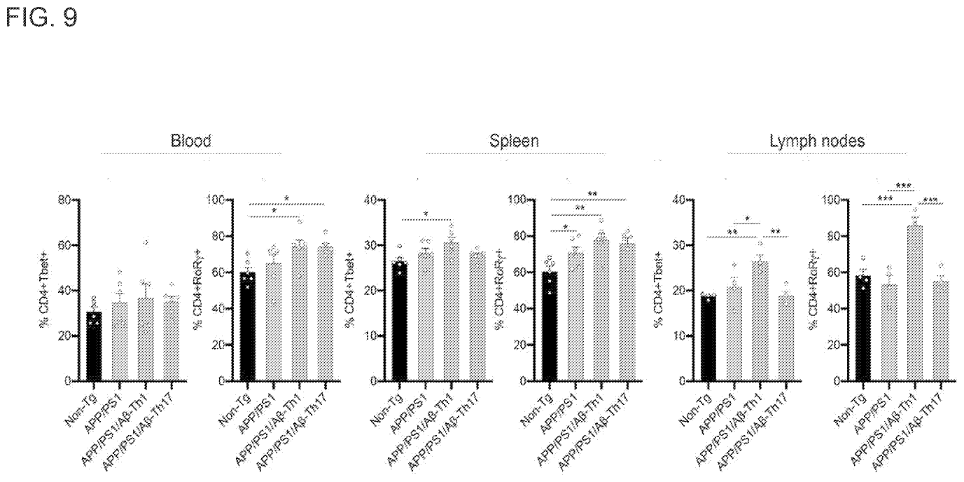

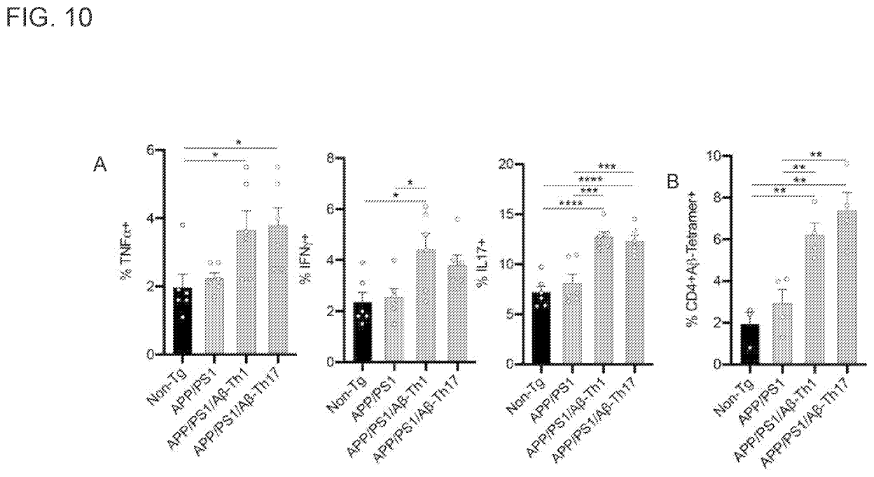

It has been demonstrated that systemic inflammatory responses onset decades before the cognitive impairment and subsequent neurodegeneration (Cunningham et al., 2015, Alzheimers Res Ther, 7, 33). Higher levels of inflammatory cytokines were observed in the blood of individuals who after 20 years demonstrated cognitive decline and decreased brain volume (Walker et al., 2017, Neurology, 89, 2262-2270). Such observations suggest that systemic inflammatory responses play an important early role in the AD pathology before amyloid deposition and microglia activation in the brain (Machhi et al., 2020, Mol Neurodegener, 15, 32; Saresella et al., 2011, Brain Behav Immun, 25, 539-547; Oberstein et al., 2018, Front Immunol, 9). To determine the effects of Aβ-Teffs on the systemic inflammation, frequency of different T cell subsets and inflammatory cytokines was measured in the blood, spleen and lymph nodes of experimental mice. Forty-two (42) days after the adoptive transfer described above, experimental mice were terminally anesthetized with pentobarbital followed by spleen isolation in complete RPMI media and blood collection via cardiac puncture in the K3EDTA tubes (Catalog no. 450475, Greiner BioOne North America). Lastly, mice were pericardially perfused with PBS and lymph nodes (axial, brachial, cervical, and inguinal) and brain were harvested. Whole blood was stained while single cell suspension was prepared from spleen and lymph nodes by mashing tissues through the 70 μm cell strainer using syringe plunger. Red blood cells were lysed from the splenocytes using the ACK lysis buffer. For all the samples, extracellular/intracellular staining was performed for flowcytometry analysis as described before (Mosely et al., 2019, Front Cell Neurosci, 13, 421; Kiyota et al., 2018, J Neuroimmunol, 319, 80-92). Briefly, either 50 μl of blood or 1×10⁶ spleen or lymph node cells were extracellularly stained with anti-mouse CD3e-PE, CD4-APC-H7, and CD8a-Pe-Cyanine 5.5antibodies for 30 min at room temperature. For intracellular staining, cells were fixed and permeabilized using transcription factor staining buffer kit for 45 min at 4° C. followed by incubation with anti-mouse Tbet-Efluor 660, RoRγ-PerCp Efluor 710 antibodies for 30 min at 4° C. The overall frequency of CD4+ and CD8+ T cells did not change across different experimental groups (FIG. 8), however the subset analysis revealed increase in pro-inflammatory markers. From the different analyzed biological compartments, CD4+Tbet+ cell frequency did not change while CD4+RORγ+ cell frequency (p<0.05) significantly increased in the spleen of the untreated APP/PS1 mice compared to the non-Tg mice (FIG. 9). In contrast, CD4+Tbet+ cells were significantly elevated in the spleen (p<0.05) and lymph node (p<0.01). This was seen in APP/PS1/Aβ-Th1 mice compared to the non-Tg counterparts (FIG. 9). Similarly, CD4+RORγ+ cell frequency significantly increased in all the tested biological compartments in the APP/PS1/Aβ-Th1 mice (p<0.01) while in case of APP/PS1/Aβ-Th17 mice, CD4+RORγ+ cell frequency significantly increased in the blood (p<0.05) and spleen (p<0.01) compared to the non-Tg mice (FIG. 9). In the lymph node cells, APP/PS1/Aβ-Th1 mice significantly increased the CD4+Tbet+ (p<0.05) and CD4+RORγ+ cells (p<0.001) compared to the APP/PS1 mice (FIG. 9). Later, it was identified that splenocytes isolated from APP/PS1/Aβ-Th1 and APP/PS1/Aβ-Th17 mice upon stimulation with PMA and ionomycin secreted significantly higher levels of pro-inflammatory cytokines TNFα (p<0.05), IFNγ (p<0.05) and IL17 (p<0.001) compared to the non-Tg and untreated APP/PS1 mice (FIG. 10A). For the intracellular cytokine analysis, 1×10⁶ spleen cells were stimulated with 20 ng/ml PMA and 1 μM ionomycin in the presence of Brefeldin A for 12 hr. After incubation, cells were surface stained with anti-mouse CD3e-PE and CD4-APC-H7 antibodies followed by intracellular staining using anti-mouse IFNγ-FITC, TNFα-eFluor 450, and IL17a-Alexa Fluor 647 antibodies. It was hypothesized that systemic inflammatory responses predominantly arise from the antigen-specific T cells present in the periphery of the experimental mice. Therefore, as a source of inflammation, frequency of Aβ reactive CD4+ T cells was determined by stimulating lymph node cells with cognate antigen (Aβ) in presence of APCs. To determine the frequency of Aβ reactive CD4+ T cells, 1×10⁶ lymph node cells were stimulated with Aβ₁₋₄₂ (25 μg/ml) in presence of feeder cells and IL2 (25 U/ml) for 5 days at 37° C. On day 5, live cells were collected by centrifugation and incubated with MHCII-IA^{b}-KLVFFAEDVGSNKGA tetramer (6 μg) for 3 hr at 37° C. After incubation, T cell-MHC-II-Aβ tetramer complex was stained with anti-mouse CD3e-PE and CD4-APC-H7 antibodies for 30 min at room temperature, followed by live-dead staining with propidium iodide (0.5 μg/ml) for 5 min at room temperature. All the samples were analyzed using BD LSR II flow cytometer and FACSDiva software (BD Biosciences) at the University of Nebraska Medical Center flow cytometry research facility. Stimulated cells from non-Tg mice showed very low binding with MHC-II-peptide tetramer suggesting presence of few Aβ-CD4+ T cells during homeostasis (FIG. 10B). In untreated APP/PS1 mice, higher frequency of Aβ-CD4+ T cells was observed compared to the non-Tg mice, although the increment was not significantly different (FIG. 10B). However, APP/PS1/Aβ-Th1 and APP/PS1/Aβ-Th17 mice exhibited significantly higher levels of Aβ-CD4+ T cells compared to the non-Tg and APP/PS1 mice (FIG. 10B, p<0.01). The results suggest that small number of adoptively transferred Aβ-Teffs have sustained antigen-recognizing CD4+ T cell population for a prolonged period of time which might contributed to systemic inflammation. Together, these results highlight the ability of disease reactive Teffs to elicit systemic pro-inflammatory environment as a contributor of AD progression.

### C. Aβ-Teffs and Treg Immunosuppression

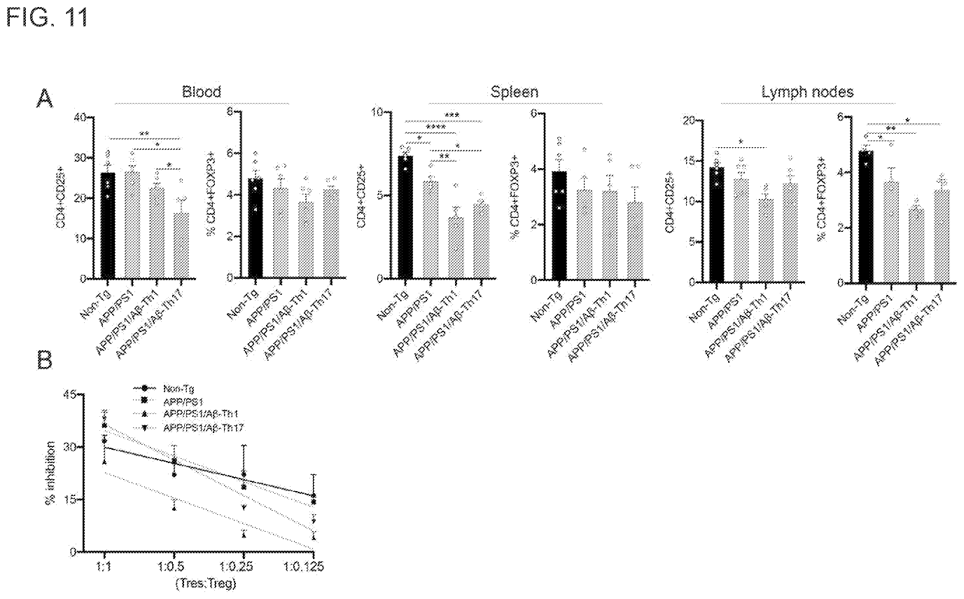

Tregs play important roles in the maintenance of immune tolerance against self and non-self antigens (J. et al., 2020, Mol Neurodegener, In press; Sakaguchi et al., 2004, Annu Rev Immunol 22, 531-562). The optimal Treg frequency is essential for the suppression of disease reactive pro-inflammatory T cell responses and such effects are attributed to their unique transcription factor FOXP3 (Hori et al., 2003, Science 299, 1057-1061; Kiyota et al., 2018, J Neuroimmunol 319, 80-92). The effects of Aβ-Teffs on the immunosuppressive and anti-inflammatory Treg frequency and function in different biological compartments was next assessed. The Treg frequency was identified using surface and intracellular markers CD4+CD25+ and CD4+FOXP3+. On day 42 post-adoptive transfer described above, mice were terminally anesthetized with pentobarbital followed by spleen isolation in complete RPMI media and blood collection via cardiac puncture in the K3EDTA tubes (Catalog no. 450475, Greiner BioOne North America). Lastly, mice were pericardially perfused with PBS and lymph nodes (axial, brachial, cervical, and inguinal) and brain were harvested. Whole blood was stained while single cell suspension was prepared from spleen and lymph nodes by mashing tissues through the 70 μm cell strainer using syringe plunger. Red blood cells were lysed from the splenocytes using the ACK lysis buffer. For all the samples, extracellular/intracellular staining was performed for flow cytometry analysis as described before (Kiyota et al., 2018, J Neuroimmunol, 319, 80-92; Mosely et al., 2019, Front Cell Neurosci, 13, 421). Briefly, either 50 μl of blood or 1×10⁶ spleen or lymph node cells were extracellularly stained with anti-mouse CD4-APC-H7, and CD25-PE-Cy7 (Catalog no. 25025182, eBioscience) antibodies for 30 min at room temperature. For intracellular staining, cells were fixed and permeabilized using transcription factor staining buffer kit for 45 min at 4° C. followed by incubation with the anti-mouse FOXP3-AlexaFluor 488 (Catalog no. 320012, BioLegend) antibody for 30 min at 4° C. APP/PS1 mice showed decreased frequencies of CD4+CD25+ cells (p<0.05) in the spleen and CD4+FOXP3+ cells (p<0.05) in the lymph node when compared to non-Tg mice (FIG. 11A). More rapid Treg decline was observed in the APP/PS1/Aβ-Th1 and APP/PS1/Aβ-Th17 mice. In the APP/PS1/Aβ-Th1 mice, CD4+CD25+ cell frequency significant decreased in the spleen (p<0.001) and lymph nodes (p<0.05) while CD4+FOXP3+ cell frequency decreased in the lymph nodes compared to the non-Tg mice (FIG. 11A, p<0.01). In the spleen, APP/PS1/Aβ-Th1 mice also showed decreased CD4+CD25+ cells compared to the APP/PS1 mice (p<0.001). The frequency of CD4+CD25+ cells significantly decreased in the blood of APP/PS1/Aβ-Th17 mice compared to the non-Tg mice, APP/PS1 and APP/PS1/Aβ-Th1 mice (FIG. 11A, p<0.05). Treg frequency does not always correlated with their immunosuppressive functions (Viglietta et al., 2004, J Exp Med, 199, 971-979; Saunders et al., 2012, J Neuroimmune Pharmacol, 7, 927-938). Therefore, to better understand the effects of Aβ-Teffs on the Treg, immunosuppressive function of Treg on the proliferation of T responder cells (Tres) was assessed in different experimental mice using CFSE assay (FIG. 11B). CD4+CD25+ Treg and CD4+CD25-Tres were isolated from the mice spleen as described earlier²⁸ using EasySep™ mouse Treg enrichment kit (Catalog no. 18783, Stemcell Technologies) as per the manufacturer's instructions. Briefly, single cell suspension was prepared by gently mashing the spleen tissue through 70 μm cell strainer using the syringe plunger, followed by red blood cell lysis using the ACK lysis buffer. CD4+ T cells were first enriched by negative selection using the EasySep™ mouse CD4+ T cell isolation cocktail from which CD25+ cells then enriched by positive selection using EasySep™ mouse CD25+ Treg selection cocktail. The isolated CD4+CD25+ cells were more than 95% pure as determined by the flow cytometry analysis. The CD4+CD25-Tres collected from the non-Tg mice spleen were used in the proliferation assay after labelling with carboxyfluorescein succinimidyl ester (CFSE) (Catalog no. C34554, Thermo Fisher Scientific). Tregs from different experimental groups were serially diluted in the U-bottom 96-well plate to obtain 50, 25, 12.5, and 6.25×10³ Tregs in 100 μl of media followed by addition of 50×10³ CFSE-labelled Tres into each well to obtain Tres:Treg rations of 1:1, 1:0.5, 1:0.25 and 1:0.125, while wells with only Tres served as controls. The mouse T cell activating CD3/CD28 Dynabeads (Catalog no. 11456D, Thermo Fisher Scientific) were added to each well at the ratio of bead:Tres (1:1) to induce Tres proliferation. The immunosuppressive functions of Treg to inhibit proliferation of CFSE stained Tres was determine after 72 h incubation at 37° C. using flow cytometry analysis. The early-stage APP/PS1 mice showed slight increase (14%) in the Treg function compared to the non-Tg mice at higher concentration (1:1) which might be due to improved Treg function during the early disease stage (Beers et al., 2011, Brain, 134, 1293-1314). However, the APP/PS1/Aβ-Th1 mice showed significantly higher Treg dysfunction compared to the non-Tg mice at all the tested concentrations. Similar Treg dysfunction was observed in the APP/PS1/Aβ-Th17 mice at lower concentrations compared to the non-Tg and untreated APP/PS1 mice. Overall, results indicated that Aβ-Th1 cells and to the lesser extend Aβ-Th17 cells increased systemic pro-inflammatory environment by downregulating Treg frequency and function leading to the breakdown of immune tolerance (Baruch et al., 2015, Nat Commun, 6, 7967).

### D. Th1 CNS Responses

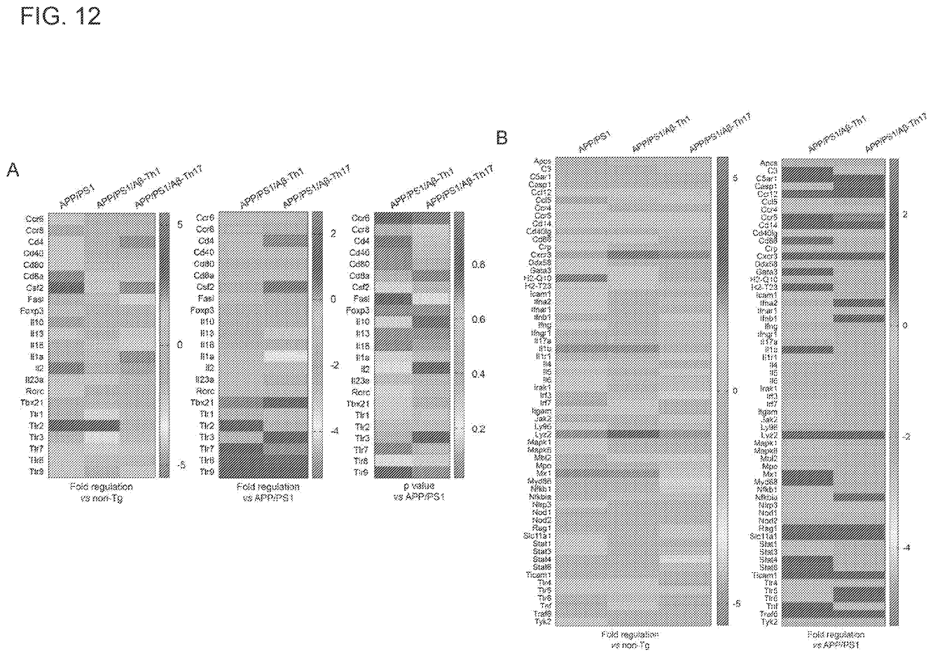

The frequency of CNS patrolling Treg is too low to isolate for the analysis (Korn et al., 2017, Nat Rev Immunol, 17, 179-194; Korin et al., 2017, Nat Neurosci, 20, 1300-1309). Therefore, the expression of different innate and adaptive immune genes was assessed using transcriptomic analysis of the RNA isolated from the hippocampus brain tissue of different experimental mice. Hippocampal tissue was isolated from mouse brain and total RNA extracted with the RNAeasy mini kit (Catalog no. 74104, Qiagen). Recovered RNA was reverse transcribed into complementary DNA (cDNA) using a RevertAid First Strand cDNA Synthesis kit (Catalog no. K1622, Thermo Fisher Scientific). One μg cDNA was amplified using primer mix from the RT²-PCR array for Mouse Innate and Adaptive Immune Responses (Catalog no. 330231, Qiagen). Quantitative RT-PCR was performed using Mastercycler Realplex EP (Eppendorf) and data were analyzed using RT² Profiler PCR array web-based data analysis software (Qiagen). Gene network analysis was performed using Ingenuity Pathway Analysis (IPA, Qiagen). Surprisingly, although not significant, the gene expression of CD4 and CD8α decreased in the brain of APP/PS1/Aβ-Th1 and APP/PS1/Aβ-Th17 mice compared to the non-Tg and untreated APP/PS1 mice (FIG. 12A). However, the gene expression of Treg transcription factor Foxp3, its anti-inflammatory cytokines Il10 and Il13 and chemokine Ccr8 significantly decreased in the APP/PS1/Aβ-Th1 and APP/PS1/Aβ-Th17 mice brain compared to the non-Tg and untreated APP/PS1 mice, suggesting that peripherally administered Aβ-Teffs have ability to affect Treg not only in the periphery but also in the CNS. Other inflammatory cytokine genes Il5, Il6, Ifng decreased while Tnf increased non-significantly in the APP/PS1/Aβ-Th1 mice compared to the APP/PS1 mice (FIG. 12B). Importantly, Tbet encoding gene Tbx21 expression increased in the APP/PS1/Aβ-Th1 (>2 fold) and APP/PS1/Aβ-Th17 mice compared to the APP/PS1 mice, while RoRγ encoding gene Rorc expression decreased compared to the non-Tg mice, highlighting that Th1 phenotype predominant immune responses were operative in the Aβ-Teffs treated AD mice brain principally in the APP/PS1/Aβ-Th1 mice.

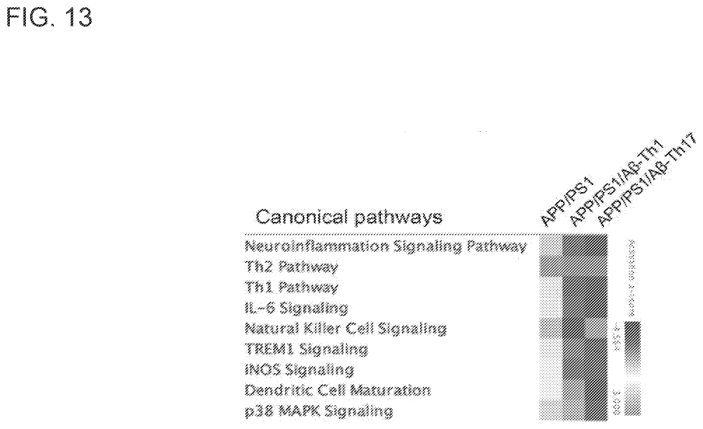

The IPA analysis of the dataset further revealed the top canonical pathways affected by the Aβ-Teffs (FIG. 13). Compared to the non-Tg mice, untreated APP/PS1 mice showed upregulated neuroinflammation, Th2, and TREM1 signaling pathways while in contrast these pathways were reduced in the APP/PS1/Aβ-Th1 and APP/PS1/Aβ-Th17 mice (FIG. 13). As per the IPA, optimum neuroinflammation plays a key role in the maintenance of CNS homeostasis by clearing the neuronal damage. Therefore, slight upregulation of the neuroinflammation signaling pathway (Z score=1.508) in the untreated APP/PS1 mice compared to the non-Tg mice suggest neuronal homeostasis maintenance in the brain while in contrast the APP/PS1/Aβ-Th1 (Z score=−2.897) and APP/PS1/Aβ-Th17 (Z score=−3.618) mice showed impaired CNS homeostasis. However, “molecular activity predictor” in the IPA showed amyloid deposition and neuronal damage in all the APP/PS1 mice groups compared to the non-Tg mice. Th2 subsets exhibit anti-inflammatory phenotype and are known to reduce inflammatory functions in the microglia cells and thereby offer neuroprotective responses (Appel et al., 2009, J Clin Invest, 119, 13-15). Upregulated Th2 signaling in the untreated APP/PS1 mice revealed Th2 driven neuroprotective responses operative during the early disease stage (FIG. 13). However, such neuroprotective Th2 signaling was significantly reduced in the APP/PS1/Aβ-Th1 and APP/PS1/Aβ-Th17 mice (FIG. 13). Recently, Jiang et al., identified that TREM1 signaling is essential for the microglial phagocytosis of Aβ which is an essential mechanism of amyloid clearance from the brain (Jiang et al., 2016, Acta Neuropathol, 132, 667-683). Treatment of Aβ-Teffs significantly reduced TREM1 signaling in the APP/PS1 mice brain compared to the non-Tg mice suggesting impaired microglial phagocytosis in these groups which may lead to the amyloid deposition in the brain.

### Example 3 Assessment of the Role of Aβ-Th1 Cells in Features Associated with AD Progression

### A. Aβ-Th1 Cells Facilitate Amyloid Deposition

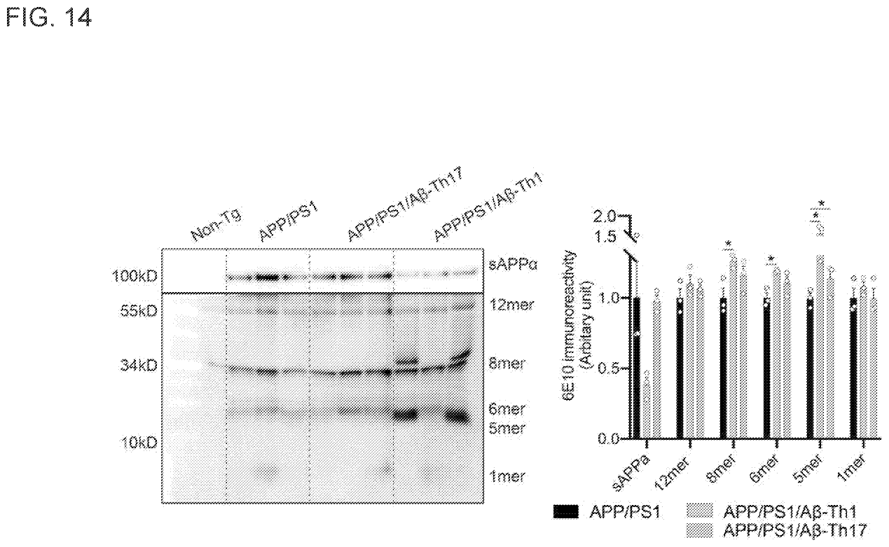

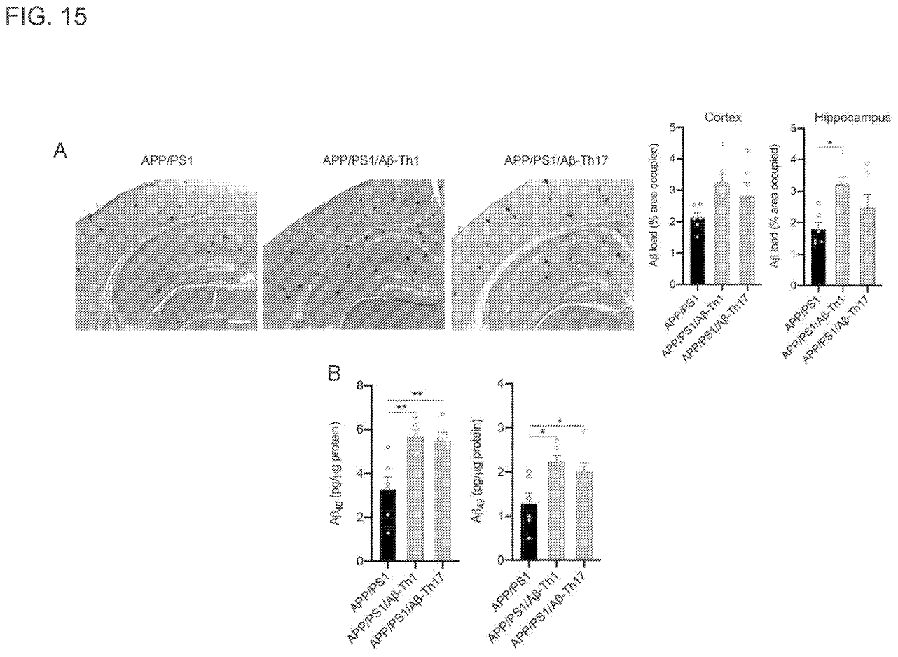

Amyloid plaque forms upon aggregation of different Aβ oligomers which are toxic and contribute to the memory impairment (Kiyota et al., 2018, J Neuroimmunol, 319, 80-92; Monsonego et al., 2003, J Clin Invest, 112, 415-22). Therefore, the expression of Aβ oligomers was studied using immunoprecipitation analysis of cortical tissue proteins. Brain cortical tissues were homogenized using lysis buffer containing 50 mM Tris-HCl (pH 8.0), 150 mM NaCl, 50 mM EDTA, 1% Triton X-100 and protease/phosphatase inhibitor mixture and centrifuged at 20,000×g for 20 min at 4° C. The supernatant was collected to quantify total protein concentration using micro BCA kit (Catalog no. 23235, Thermo Fisher Scientific). Immunoprecipitation was performed according to prior protocols^{6, 24}. Briefly, 100 μg of protein was incubated with unconjugated pan-Aβ antibody (2 μg/ml) (6E10, rabbit monoclonal, Catalog no. 803001, Biolegend,) in radioimmunoprecipitation assay (RIPA) buffer containing 25 mM Tris-HCl (pH 7.6), 150 mM NaCl, 1% NP-40, 1% sodium deoxycholate, 0.1% sodium dodecyl sulfate (SDS) and protease inhibitor (Catalog no. 89901, Thermo Fisher Scientific,) at 4° C. for 1 hr, followed by incubation with 40 μl Protein A/G Plus agarose (Catalog no. sc2003, Santa Cruz Biotechnology,) at 4° C. overnight. Precipitates were collected by centrifugation at 3000 rpm for 5 min, reconstituted with Laemmli buffer, incubated at 95° C. for 5 min, and then analyzed by electrophoresis on 16% SDS-polyacrylamide Tris-Tricine gel. Proteins were transferred onto the 0.2 μm pore size polyvinylidine fluoride membrane (Immobilon-P, Catalog no. IPVH00010, Millipore). Membranes were blocked in 3% BSA/Tris-buffered saline Tween 20 (TBST) and incubated with biotinylated 6E10 monoclonal antibody (1:1000; Catalog no. 803001, Biolegend), followed by incubation with HRP-conjugated streptavidin (Catalog no. N100, ThermoFisher Scientific). The expression of non-amyloidogenic and neuroprotective APP fragment, soluble APPα (sAPPα) decreased while in contrast, the levels of amyloidogenic and neurotoxic Aβ oligomers 8-mer, 6-mer and 5-mer significantly increased in the brain of APP/PS1/Aβ-Th1 mice compared to the untreated APP/PS1 mice (FIG. 14, p<0.05). APP/PS1/Aβ-Th17 mice also showed increased levels of 8 mer (16%), 6 mer (10%) and 5 mer (14%) compared to the APP/PS1 mice, with non-significant difference. APP/PS1 mice develop amyloid plaque in the brain as early as three months of age (Faridar et al., 2020, Brain Commun, 2 fcaa112; Iliff et al., 2012 Sci Transl Med, 4, 47ra111). Therefore, immunohistochemistry was next used to determine the effects of Aβ-Teffs on amyloid plaque deposition in the brain of different experimental mice. After transcardial perfusion, mouse brains were immediately harvested and divided into two hemispheres. The left was immediately frozen on dry ice for biochemical analysis and right was immersed in freshly depolymerized 4% paraformaldehyde in 1×PBS for 48 hr at 4° C. and cryoprotected by immersion in 15%, then 30% sucrose for 24 hr at 4° C. Fixed brains were sectioned coronally with a Cryostat (ThermoFisher) with sections serially collected and stored at −80° C. Immunohistochemistry was performed using antibodies against pan-Aβ (1:500, rabbit polyclonal, Catalog no. 715800, Thermo Fisher Scientific). For immunodetection, biotin-conjugated anti-rabbit was used followed by a tertiary incubation with Vectastain ABC Elite kit (Catalog no. PK6100, Vector Laboratories). Slides were masked and coded, and the Aβ occupied area was calculated using Cavalieri estimator probe (grid spacing 15 μm). Similar to the Aβ oligomers, amyloid plaque load increased in both cortex (p=0.063) and hippocampus (p<0.05) regions of the APP/PS1/Aβ-Th1 mice brain compared to the untreated APP/PS1 mice (FIG. 15A). Lastly, the expression of soluble human Aβ₄₀ and Aβ₄₂ levels in the brain lysates of different experimental mice were determined using ELISA. Mouse cortical tissues were homogenized using 50 mM Tri-HCl (pH 7.6) containing 150 mM NaCl and protease/phosphatase inhibitor mixture and centrifugation at 200,000×g for 20 min at 4° C. The supernatant was subjected to human Aβ₄₀ and Aβ₄₂ ELISA kits (Catalog no. KHB3482 and KHB3442, Thermo Fisher Scientific) according to the manufacturer's instructions. As shown in FIG. 15B, the soluble Aβ₄₀ levels significantly increased in the APP/PS1/Aβ-Th1 and APP/PS1/Aβ-Th17 mice compared to the untreated APP/PS1 mice (p<0.01). Similarly, Aβ₄₂ levels significantly increased the APP/PS1/Aβ-Th1 (p<0.05) and APP/PS1/Aβ-Th1 (p<0.05) mice brain compared to the APP/PS1 mice.

### B. Aβ-Th1 Cells Activate Microglia

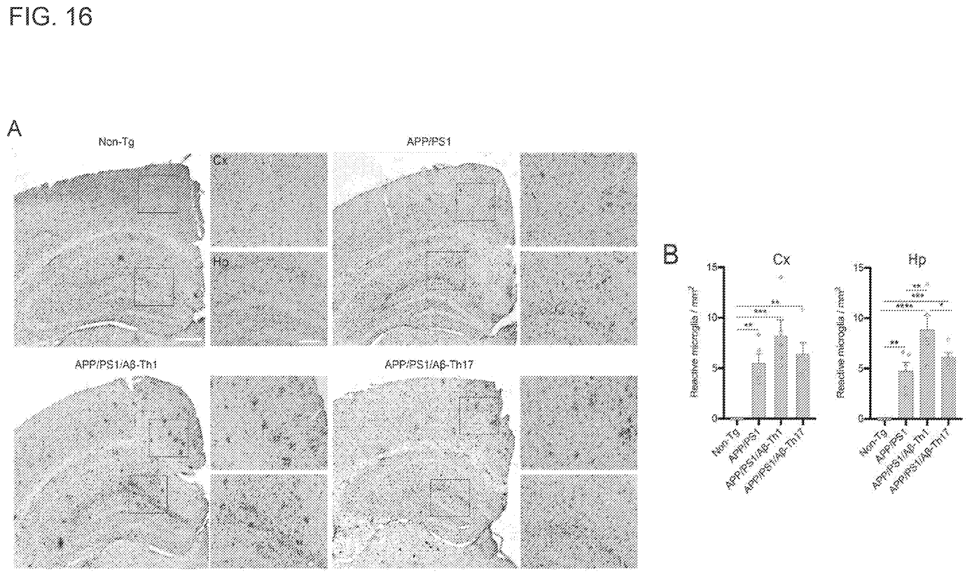

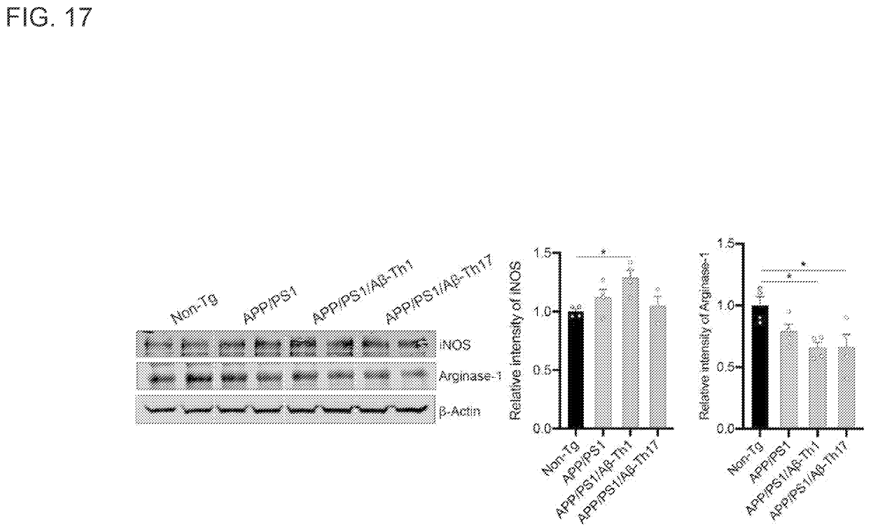

Microglia activation is a unique signature of neuroinflammation observed in AD patients and animal models (Kinney et al., 2018, Alzheimers Dement (N Y), 4, 575-590, Zenaro et al., 2015, Nat Med, 21, 880-886). To determine the effects of Aβ-Teffs on microglial responses, Iba1 reactive cells were counted in the brain regions of experimental mice. After transcardial perfusion, mouse brains were immediately harvested and divided into two hemispheres. The left was immediately frozen on dry ice for biochemical analysis and right was immersed in freshly depolymerized 4% paraformaldehyde in 1×PBS for 48 hr at 4° C. and cryoprotected by immersion in 15%, then 30% sucrose for 24 hr at 4° C. Fixed brains were sectioned coronally with a Cryostat (ThermoFisher) with sections serially collected and stored at −80° C. Immunohistochemistry was performed using antibodies against Iba1 (1:1000, rabbit polyclonal, Catalog no. 01919741, Wako). For immunodetection, biotin-conjugated anti-rabbit was used followed by a tertiary incubation with Vectastain ABC Elite kit (Catalog no. PK6100, Vector Laboratories). Slides were masked and coded and number of Iba1 reactive cells were counted using Optical Fractionator module of Stereo Investigator system (MBF Bioscience) as described earlier (Kiyota et al., 2018, J Neuroinflammation, 15, 137). Briefly, a high-sensitivity digital camera (OrcaFlash2.8, Hamamatsu C11440-10C, Hamamatsu, Japan) interfaced with a Nikon Eclipse 90i microscope (Nikon, Melville, NY, USA) was used. Within the Stereo Investigator program, the contour in each section was delineated using a tracing function. While sections showed tissue shrinkage along the anteroposterior axis, the extent of shrinkage between different animals was similar. The dimensions for the counting frame (120×100 μm) and the grid size (245× 240 μm) were set. The z-plane focus was adjusted at each section for clarity. Cells were quantified by the fractionator with marked positive cells in each counting frame. Based on the set parameters and marked cell counts, the Stereo Investigator program computed the estimated cell populations which were compared between groups. As shown in FIG. 16A, untreated APP/PS1 mice developed higher Iba1 positive (Iba1+) cell clumps compared to the non-Tg mice suggesting microglial activation in the early-stage AD mice. However, APP/PS1/Aβ-Th1 mice showed worsened microgliosis as evidenced by increased microglial aggregates in both cortex and hippocampus regions of the brain compared to the non-Tg mice (FIG. 16A). Stereological quantification revealed significantly increased number of Iba1+ reactive microglia cells in the cortex (p<0.01) and hippocampus (p<0.01) of untreated APP/PS1 mice brain compared to the non-Tg mice (FIG. 16B). However, APP/PS1/Aβ-Th1 mice showed higher microglia activation in the cortex and hippocampus (p<0.01) even compared to the APP/PS1 mice (FIG. 16B). Although non-significant, APP/PS1/Aβ-Th17 mice also showed upregulated reactive microglia count in the cortex (28%) and hippocampus (47%) brain regions compared to the APP/PS1 mice (FIG. 16B). To get more insights into the microglia activation state, the expression of iNOS and arginase-1 assessed using western blot analysis which represents pro- and anti-inflammatory microglia phenotypes respectively (Kiyota et al., 2018, J Neuroinflammation, 15, 137; Kiyota et al., 2018, J Neuroimmunol, 319, 80-92). For Western blot analysis, 80 μg or 20 μg of tissue protein was incubated with β-mercaptoethanol containing Laemmli buffer at 100° C. for 5 min, followed by electrophoresis on SDS-polyacrylamide gel and transferred to polyvinylidene fluoride membrane. The membranes were blocked in 5% skim milk/TBST and incubated with primary antibodies to arginase 1 (1:300, Catalog no. 93668S, Cell Signaling Technology), inducible nitric oxide synthase (iNOS) (1:300, Catalog no. 13120S, Cell Signaling Technology), and β-actin (1:2000, Catalog no. A3854, Sigma) at 4° C. overnight, followed by 60 min incubation in 5% skim milk/TBST with horseradish peroxidase-conjugated anti-rabbit, or mouse secondary antibodies (1:2000, Santa Cruz Biotechnology). Immunoreactive bands were detected using SuperSignal West Pico or Femto Chemiluminescent substrate, and images were captured using an iBlot750 Imager (Thermo Fisher Scientific). Immunoblots were quantified using ImageJ software (NIH) relative to β-actin expression. The immunoblot analysis revealed upregulation of iNOS and decreased Arginase-1 in the early-stage APP/PS1 mice brain compared to the non-Tg mice. However, APP/PS1/Aβ-Th1 mice significantly increased the expression of iNOS in the brain compared to the non-Tg mice (FIG. 17, p<0.05). In contrast, arginse-1 expression significantly reduced in the brain of APP/PS1/Aβ-Th1 and APP/PS1/Aβ-Th17 mice compared to the non-Tg mice (FIG. 17, p<0.05). These results revealed the ability of Aβ-Teffs to activate microglia and alter their cellular phenotype in the AD mice brain with more pronounced effects observed with Aβ-Th1 cells.

### C. Aβ-Th1 Cells Affect Neuronal Progenitor Cell Numbers and Function

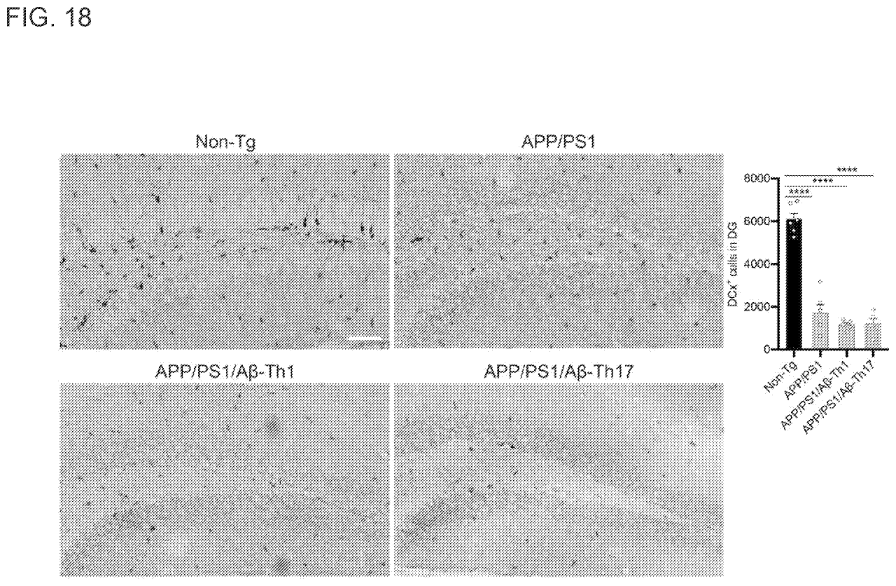

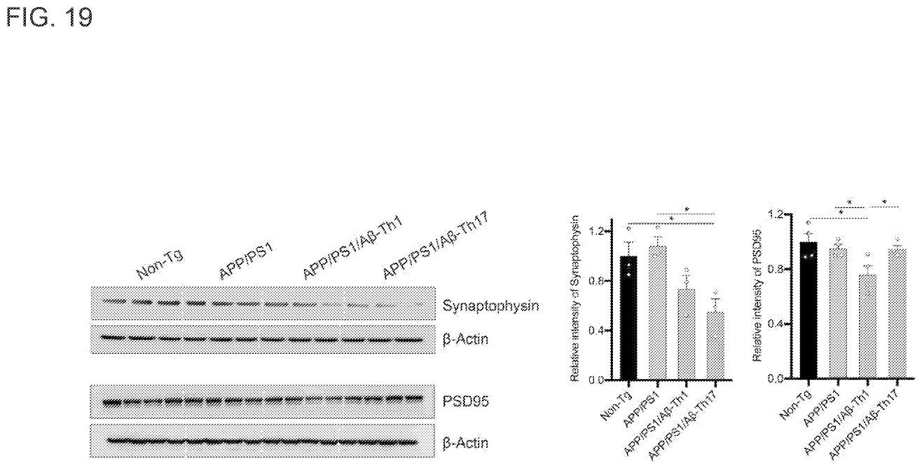

Hippocampal neurogenesis can be correlated with memory function and neural plasticity, all affected by AD progression (Kiyota et al., 2015, Brain Behav Immun, 49, 311-321; Deng et al., 2010, Nat Rev Neurosci, 11, 339-350). Doublecortin (Dcx) is a marker of neuronal progenitors used to examine neurogenesis in the dentate gyrus region of the hippocampus (Deng et al., 2010, Nat Rev Neurosci, 11, 339-350, Kiyota et al., 2018, J Neuroimmunol 319, 80-92). After transcardial perfusion, mouse brains were immediately harvested and divided into two hemispheres. The left was immediately frozen on dry ice for biochemical analysis and right was immersed in freshly depolymerized 4% paraformaldehyde in 1×PBS for 48 hr at 4° C. and cryoprotected by immersion in 15%, then 30% sucrose for 24 hr at 4° C. Fixed brains were sectioned coronally with a Cryostat (ThermoFisher) with sections serially collected and stored at −80° C. Immunohistochemistry was performed using antibodies to doublecortin (Dcx) (1:500, goat polyclonal, Catalog no. Sc8066, Santa Cruz Biotechnology). For immunodetection, anti-goat IgG secondary antibody was used followed by a tertiary incubation with Vectastain ABC Elite kit (Catalog no. PK6100, Vector Laboratories). Slides were masked and coded and number of Dcx positive (Dcx+) neuroprogenitor cells were counted using the Optical Fractionator module of Stereo Investigator system (MBF Bioscience) as described earlier (Kiyota et al., 2018, J Neuroinflammation, 15, 137). Briefly, a high-sensitivity digital camera (OrcaFlash2.8, Hamamatsu C11440-10C, Hamamatsu, Japan) interfaced with a Nikon Eclipse 90i microscope (Nikon, Melville, NY, USA) was used. Within the Stereo Investigator program, the contour in each section was delineated using a tracing function. While sections showed tissue shrinkage along the anteroposterior axis, the extent of shrinkage between different animals was similar. The dimensions for the counting frame (120×100 μm) and the grid size (245× 240 μm) were set. The z-plane focus was adjusted at each section for clarity. Cells were quantified by the fractionator with marked positive cells in each counting frame. Based on the set parameters and marked cell counts, the Stereo Investigator program computed the estimated cell populations which were compared between groups. It was found that Dcx+ cell frequency significantly decreased in the dentate gyrus region of the untreated APP/PS1 mice brain compared to the non-Tg mice (p<0.001, FIG. 18). However, the Aβ-Teffs were unable to further impair the neurogenesis in the APP/PS1 mice (FIG. 18). However, in the hippocampus areas outside the dentate gyrus, Dcx+ neuronal staining decreased in the APP/PS1/Aβ-Th1 and APP/PS1/Aβ-Th17 mice compared to both non-Tg and APP/PS1 mice (not quantified). Next, pre- and post-synaptic protein (synaptophysin and PSD95, respectively) expression in cortical brain lysates were assessed using western blot analysis. For functional plasticity, co-expression of both pre- and postsynaptic proteins is essential and alteration in either key protein levels affect neuronal plasticity and memory functions (Shi et al., 2017, Sci Transl Med, 9; Kiyota et al., 2018, J Neuroimmunol 319, 80-92). For western blot analysis, 80 μg or 20 μg of tissue protein was incubated with β-mercaptoethanol containing Laemmli buffer at 100° C. for 5 min, followed by electrophoresis on SDS-polyacrylamide gel and transferred to polyvinylidene fluoride membrane. The membranes were blocked in 5% skim milk/TBST and incubated with primary antibodies to synaptophysin (1:1000, Catalog no. MAβ5258, Millipore), PSD95 (1:1000, Catalog no. ab18258, Abcam) and β-actin (1:2000, Catalog no. A3854, Sigma) at 4° C. overnight, followed by 60 min incubation in 5% skim milk/TBST with horseradish peroxidase-conjugated anti-rabbit, or mouse secondary antibodies (1:2000, Santa Cruz Biotechnology). Immunoreactive bands were detected using SuperSignal West Pico or Femto Chemiluminescent substrate, and images were captured using an iBlot750 Imager (Thermo Fisher Scientific). Immunoblots were quantified using ImageJ software (NIH) relative to β-actin expression. The immunoblot quantification revealed unaltered expression of synaptophysin and PSD95 in the untreated APP/PS1 mice compared to the non-Tg mice (FIG. 19). However, APP/PS1/Aβ-Th1 (p=0.320) and APP/PS1/Aβ-Th17 (p<0.05) mice showed reduced expression of synaptophysin compared to non-Tg mice. Additionally, APP/PS1/Aβ-Th1 (p<0.05) but not APP/PS1/Aβ-Th17 mice showed reduced expression of PSD95 compared to the non-Tg mice. Thus, only presynaptic protein abnormalities were observed in the APP/PS1/Aβ-Th17 mice, while both pre- and postsynaptic protein densities were affected in the APP/PS1/Aβ-Th1 mice. Overall, more pronounced detrimental effects were observed after the Aβ-Th1 cells treatment in the APP/PS1 mice confirming their superior pathological phenotype in the amyloid rich environment compared to the Aβ-Th17 subtypes.

FIG. 20 depicts an exemplary lentiviral vector for delivery of beta-amyloid specific TCR sequences, such as for use in engineering cells, e.g., T cells, such as Tregs, for use in the treatment, diagnosis, or monitoring of Alzheimer's Disease.

### D. Discussion

Previous studies demonstrated a spectrum of effects by Aβ-reactive Teffs in animal models and AD patients (McQuillan et al., 2010, Brain Behav Immun, 24, 598-607; Mittal et al., 2019, iScience, 16, 298-311; Nicoll et al., 2003, Nat Med, 9, 448-452; Monsonego et al., 2006, Proc Natl Acad Sci USA, 103, 5048-5053). However, such short-term maintained polyclonal Teffs are not the best choice to study AD underlying mechanisms of action. Therefore for these studies, stable monoclonal Aβ-specific Th1 and Th17 cells (Aβ-Th1 and Aβ-Th17, respectively) were generated without compromising cytokine and antigen specificity even after prolonged culture (Machhi et al., 2021, J Neuroinflammation, 18, 272). Adoptive transfer of Aβ-Th1 and Aβ-Th17 cells enhanced memory impairment and amyloid deposition in APP/PS1 mice. The results are inconsistent with the previous report where only IFNγ secreting Aβ-Th1 cells produced detrimental cognitive effects in the APP/PS1 mice (Browne et al., 2013, J Immunol, 190, 2241-225). One possible explanation, without being limited to this explanation, is that unstable, short-term effector T cell lines not monoclonnally developed as in other reports might have acquired combined phenotypes of Th1 and Th17 cells via IFNγ and IL12 signaling (Lexberg et al., 2010, Eur J Immunol, 40, 3017-3027; Muranski et al., 2013, Blood, 121, 2402-2414). Inconsistent with these results, clinical studies have shown increased frequency of IFNγ secreting Th1- (Monsonego et al., 2003, J Clin Invest, 112, 415-422) and IL17-secreting Th17 cells (Saresella et al., 2011, Brain Behav Immun, 25, 539-547; Oberstein et al., 2018, Front Immunol, 9, 1213) in early-stage AD patients compared to healthy controls. Neurotoxic effects of antigen-specific Th17 cells in Parkinson's disease (PD) animal models (Reynolds et al., 2010, J Immunol, 184, 2261-2271) and patients (Saunders et al., 2012, J Neuroimmune Pharmacol, 7, 927-938; Sommer et al., 2018, Cell Stem Cell, 23, 123-131 e126; Sommer et al., 2019, Cell Stem Cell, 24, 1006) have been previously demonstrated. Indeed, peripheral adaptive immune impairments are comparable between two most common neurodegenerative diseases, AD and PD patients (Saunders et al., 2012, J Neuroimmune Pharmacol, 7, 927-938; Faridar et al., 2020, Brain Commun, 2, fcaa112; Brosseron et al., 2020, Alzheimers Demen,t 16, 292-304; Gendelman et al., 2017, NPJ Parkinsons, Dis 3, 10), it is speculated that disease operative Th17 cells can be lethal although to a lesser extend compared to the Th1 cells in AD pathogenesis.

Systemic inflammation initiates in AD patients, early before the onset of cognitive impairment suggesting indispensable role of peripheral immune activation in AD pathogenesis (Oberstein et al., 2018, Front Immunol, 9, 1213; Cunningham et al., 2015, Alzheimers Res Ther, 7, 33; Walker et al., 2017, Neurology, 89, 2262-2270). During early disease stage, Aβ drains from the CNS to peripheral cervical lymph nodes (Iliff et al., 2012, Sci Transl Med, 4, 147ra111) where APCs process and present Aβ to peripheral T cells via MHCI/II, processes vital to initiate peripheral adaptive immune activation (Weller et al., 2009, Acta Neuropathol, 117, 1-14; Machhi et al., 2020, Mol Neurodegener, 15, 32; Anderson et al., 2014, Transl Neurodegener, 3, 25). However, with disease progression, compromised Aβ lymphatic drainage leads to formation of cerebral amyloid angiopathy and Aβ accumulation in the brain parenchyma (Da Mesquita et al., 2018, Nature, 560, 185-191; Rasmussen et al., 2018, Lancet Neurol, 17, 1016-1024). Meanwhile, peripheral clonally expanded and activated T cells expressing chemokines (CCL3, CCL4) cross the compromised bold-brain-barrier (BBB) and affect resident microglia and neuronal cells contributing neuroinflammation (Man et al., 2007, Neurobiol Aging, 28, 485-496; Schrum et al., 1996, J Immunol 157, 3598-3604). Recently, Gate et al showed elevated frequency of CD8+ effector memory T cells in the blood of AD patients were negatively correlated with the cognitive functions. Peripheral CD8+ Teffs invaded the CNS and clonally expand into the cerebrospinal fluid to contribute neuroinflammation (Gate et al., 2020, Nature, 577, 399-404). Here, adoptive transfer of Aβ-Th1 and Aβ-Th17 cells into the APP/PS1 mice increased the frequency of antigen-specific T cells in the periphery evidenced by higher expression of TNFα, IFNγ and IL-17 secreting and MHC-II-peptide tetramer binding CD4+ T cells. Elevated Th1 and Th17 cytokines can weaken the tight junctions of the BBB to allow peripheral inflammatory mediators including TNFα, IL-1β, and IL-6 to extravasate into the AD brain (Heneka et al., 2010, J Neural Transm (Vienna), 117, 919-947; Huppert et al., 2010, FASEB J, 24, 1023-1034).

Previous studies reported decreased frequency and function of anti-inflammatory and immunosuppressive CD4+CD25+FOXP3+ Treg in blood of AD patients (Ciccocioppo et al., 2019, Sci Rep, 9, 8788; Beers et al., 2011, Brain, 134, 1293-1314) and experimental mouse models of human disease (Kiyota et al., 2018, J Neuroimmunol, 319, 80-92). Additionally, transient breaking of immune tolerance by Treg depletion is associated with amyloid clearance and neuroinflammation restoration via subsequent CNS recruitment of immunoregulatory Treg and monocyte derived macrophages (Baruch et al., 2015, Nat Commun, 6, 7967). Other reports demonstrated neuroprotective potential of polyclonal Treg adoptive transfer or expansion in AD animal models (Dansokho et al., 2016, Brain, 139, 1237-1251; Baek et al., 2016, Oncotarge,t 7, 69347-69357) and PD (Reynolds et al., 2007, J Leukoc Biol, 82, 1083-1094; Olson et al., 2015, Neurosci, 35, 16463-16478) and ALS patients (Thonhoff et al., 2018, Neurol Neuroimmunol Neuroinflamm, 5, e465). In aggregate, these reports suggest an indispensable role of Treg deficits in AD progression, but the underlying cause is unknown. For the first time, the role of disease-operative CD4+Aβ-Teff subsets in Treg dysfunction in APP/PS1 mice was identified by the studies conducted in these Examples. It was demonstrated that adoptive transfer of Aβ-Th1 and Aβ-Th17 cells significantly reduced the frequency of CD4+CD25+ and CD4+FOXP3+ Treg in the blood, spleen and LN of APP/PS1 mice. Additionally, Treg immunosuppressive function was greatly compromised in the APP/PS1 mice receiving Aβ-Th1 cells compared to the Aβ-Th17 treated or untreated APP/PS1 mice. Interestingly, the effects of Aβ-Teffs on Treg function was not limited to the periphery, but also extended to the CNS where FOXP3 is an essential transcription factor for Treg immunosuppressive function (Hayden et al., 2004, Genes Dev, 18, 2195-224), and Treg anti-inflammatory cytokines IL-10 and IL-13 expression significantly decreased in both Aβ-Th1 and Aβ-Th17 cells treated APP/PS1 mice compared to untreated APP/PS1 mice. It is speculated that antigen-specific T cells generated in vivo by the adoptively transferred Aβ-Teffs elicited Treg impairments through their secreted cytokines (Gendelman et al., 2015, J Neuroimmune Pharmacol, 10, 645-650; Machhi et al., 2020, Mol Neurodegener, 15, 32).

Microglia serve key roles in processing and presenting self-antigens including Aβ to T cells and such coordinated communication maintain immune tolerance (Machhi et al., 2020, Mol Neurodegener, 15, 32; Mittal et al., 2019, iScience, 16, 298-311). Homeostatic microglia exhibit ramified morphology and are able to clear Aβ deposits through phagocytosis. However, with disease advancement, microglia are more activated and acquire amoeboid and less ramified morphology with compromised phagocytic capabilities (Kiyota et al., 2018, J Neuroimmunol, 319, 80-92). Upon antigenic stimulation, microglia can acquire either classically activated (M1) or alternatively activated (M2) phenotype, representing pro-inflammatory and anti-inflammatory phenotypes, respectively (Kiyota et al., 2018, J Neuroimmunol, 319, 80-92; Kiyota et al., 2018, J Neuroinflammation, 15, 137). Here the expression of iNOS, an M1 marker, was significantly elevated while arginase, an M2 marker, was reduced in the brain of APP/PS1/Aβ-Th1 mice, suggesting that disease-linked CD4+ Teffs alter microglial polarization leading to worse disease outcomes. Recently, using genome-wide analysis and single-cell mass cytometry (Bottcher et al., 2019, Nat Neurosci, 22, 78-90; Jordao et al., 2019, Science, 363), identified different microglial clusters in various brain regions of human and mice which show distinct differences correlated with physiological and pathological conditions. However, the unique function associated with each microglia subset has not yet been identified. These reports suggest that microglia phenotypes are more complicated and beyond simplified M1 and M2 phenotypes (Stratoulias et al., 2019, EMBO J, 38, e101997; Ransohoff et al., 2016, Nat Neurosci, 19, 987-991). In the present study, TREM1 signaling was significantly reduced in the APP/PS1/Aβ-Th1 and APP/PS1/Aβ-Th17 mice brain compared to the non-Tg mice. A previous study has shown that TREM1 signaling is affected in the AD patients and correlated with amyloid neuropathology in the brain. Furthermore, TREM1 knockdown significantly reduced Aβ-mediated phagocytic activity of mouse primary microglia (Jiang et al., 2016, Acta Neuropathol, 132, 667-683). Therefore, it is evident that Aβ-Th1 cells worsen microglia activation and ultimately microglial phagocytic activity under the vicinity of misfolded protein.

In addition to affecting microglia responses, Aβ-Th1 cells significantly increased amyloid deposition in both cortex and hippocampus evidenced by upregulation of soluble Aβ oligomers, Aβ₄₂ and insoluble Aβ plaques compared to untreated APP/PS1 mice. Aβ-Th17 cell treatment did not significantly affect amyloid load and microglial responses in APP/PS1 mice brain possibly due to their less profound systemic inflammatory responses compared to the Aβ-Th1 cells. The results were further supported by the previous study showing Th1 biased immune responses accountable for the CD4+ Teffs detrimental effects in APP/PS1 mice (Browne et al., 2013, J Immunol, 190, 2241-225). Neuroinflammation arises either as a result of T cells driven microglia responses or through their direct cytokine release (Anderson et al., 2014, Transl Neurodegener, 3, 25; Machhi et al., 2020, Mol Neurodegener, 15, 32), ultimately affecting synaptic integrity and neurogenesis, with their demise serving as signature hallmarks of terminal neurodegeneration (Kiyota et al., 2018, J Neuroimmunol, 319, 80-92; Kiyota et al., 2018, J Neuroinflammation, 15, 137). Functional co-localization of pre- and post-synaptic proteins are essential for memory development and defects in either protein can worsen cognitive functions (Kiyota et al., 2018, J Neuroinflammation, 15, 137; Shi et al., 2017, Sci Transl Med, 9). APP/PS1/Aβ-Th1 mice showed defective pre- and post-synaptic protein expression while APP/PS1/Aβ-Th17 mice demonstrated presynaptic abnormalities. Neuronal progenitor development was affected in the dentate gyrus region of APP/PS1 mice, but was not worsened by Aβ-Teffs. However, outside dentate gyrus region, Dcx+ cells were reduced by Aβ-Teffs suggesting their contribution to detrimental effects on neurogenesis.

A previous study has demonstrated that complete loss of peripheral adaptive immune system exacerbated amyloid deposition and neuroinflammation in 5×fAD-Rag mice and reconstitution of the adaptive immune cell population restores AD pathology (Marsh et al., 2016, Proc Natl Acad Sci USA, 113:1316-1325). This is in agreement with the finding that from people living with HIV, 50% of patients develop AD linked cognitive deficits later in age despite antiretroviral therapy due, in part, to selective functional decline of peripheral CD4+ T cells (Brew et al., 2005, Neurology, 65, 1490-1492). Although, adaptive immune T cells are essential to control AD pathology, Aβ-reactive Teffs can develop meningoencephalitis as evidenced in a phase2a clinical trial (NCT00021723) that tested immunization with full length Aβ in QS21 adjuvant (Nicoll et al., 2003, Nat Med, 9, 448-452; Monsonego et al., 2006, Proc Natl Acad Sci USA, 103, 5048-5053). Indeed, CNS effects of Aβ-Teffs were studied previously in AD patients and animal models (Monsonego et al., 2006, Proc Natl Acad Sci USA, 103, 5048-5053; Mittal et al., 2019, iScience, 16, 298-311), but this is the first study characterizing the effects of stable monoclonal Aβ-Th1 and Aβ-Th17 Teffs on neuroinflammation in an AD mouse model. In these studies, Aβ-Th1 cells induced consistent pro-inflammatory responses in both periphery and CNS leading to memory impairment, increased amyloid load, microgliosis, and neurodegeneration. Tregs are identified as a key regulator of Aβ-Teff driven neuroinflammation in amyloid rich environment leading to the breakdown of immune tolerance (Machhi et al., 2020, Mol Neurodegener, 15, 32; Baruch et al., 2015, Nat Commun, 6, 7967). Therefore, it is postulated that instead of polyclonal Tregs which are associated with the risk of global immunosuppression (Zhang et al., 2015, Immunotherapy 7, 1201-1211; Hu et al., 2018, Nat Rev Neurol 14, 559-568), development of Aβ-specific Tregs by engineering Treg cells using the TCR or antigen-binding fragments thereof derived from Aβ-Th1, e.g., by including the CDR sequences and/or other sequences shown in Table E1. These engineered Treg cells would serve as a safe and efficacious therapeutic alternate for the management of elderly AD patients.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| # | SEQUENCE | ANNOTATION |
|---|---|---|
| 1 | ATGGACAAGATCCTGACAGCATCGTTTTTACTTCTAGGCCTTCACCT | TCR alpha chain, |
| | AGCTGGGGTGAGTGGCCAGCAGGAGAAACGTGACCAGCAGCAGGT | DNA |
| | GAGACAAAGTCCCCAATCTCTGACAGTCTGGGAAGGAGAGACCGCA | |
| | ATTCTGAACTGCAGTTATGAGAACAGTGCTTTTGACTACTTCCCATG | |
| | GTACCAGCAGTTCCCTGGGGAAGGTCCCGCTCTCCTGATATCCATAC | |
| | TTTCAGTGTCCAATAAAAAGGAAGATGGACGATTCACAATCTTCTTC | |
| | AATAAAAGGGAGAAAAAGCTCTCCTTGCACATTGCAGACTCTCAGC | |
| | CTGGAGACTCAGCCACCTACTTCTGTGCAGGGGGAGACTATGCAAA | |
| | CAAGATGATCTTTGGCTTGGGAACCATTTTGAGAGTCAGACCTCACA | |
| | TCCAGAACCCAGAACCTGCTGTGTACCAGCTGAAAGATCCGCGCAG | |
| | CCAGGATAGCACCCTGTGCCTGTTTACCGATTTTGATAGCCAGATTA | |
| | ACGTGCCGAAAACCATGGAAAGCGGCACCTTTATTACCGATAAAAC | |
| | CGTGCTGGATATGAAAGCGATGGATAGCAAAAGCAACGGCGCGATT | |
| | GCGTGGAGCAACCAGACCAGCTTTACCTGCCAGGATATTTTTAAAGA | |
| | AACCAACGCGACCTATCCGAGCAGCGATGTGCCGTGCGATGCGACC | |
| | CTGACCGAAAAAAGCTTTGAAACCGATATGAACCTGAACTTTCAGA | |
| | ACCTGAGCGTGATGGGCCTGCGCATTCTGCTGCTGAAAGTGGCGGG | |
| | CTTTAACCTGCTGATGACCCTGCGCCTGTGGAGCAGC | |
| 2 | MDKILTASFLLLGLHLAGVSGQQEKRDQQQVRQSPQSLTVWEGETAIL | TCR alpha chain, |
| | NCSYENSAFDYFPWYQQFPGEGPALLISILSVSNKKEDGRFTIFFNKREK | amino acid |
| | KLSLHIADSQPGDSATYFCAGGDYANKMIFGLGTILRVRPHIQNPEPAV | |
| | YQLKDPRSQDSTLCLFTDFDSQINVPKTMESGTFITDKTVLDMKAMDSK | |
| | SNGAIAWSNQTSFTCQDIFKETNATYPSSDVPCDATLTEKSFETDMNLN | |
| | FQNLSVMGLRILLLKVAGFNLLMTLRLWSS | |
| 3 | ATGGGCTCCATTTTCCTCAGTTGCCTGGCCGTTTGTCTCCTGGTGGCA | TCR beta chain, |
| | GGTCCAGTCGACCCGAAAATTATCCAGAAACCAAAATATCTGGTGG | DNA |
| | CAGTCACAGGGAGCGAAAAAATCCTGATATGCGAACAGTATCTAGG | |
| | CCACAATGCTATGTATTGGTATAGACAAAGTGCTAAGAAGCCTCTAG | |
| | AGTTCATGTTTTCCTACAGCTATCAAAAACTTATGGACAATCAGACT | |
| | GCCTCAAGTCGCTTCCAACCTCAAAGTTCAAAGAAAAACCATTTAGA | |
| | CCTTCAGATCACAGCTCTAAAGCCTGATGACTCGGCCACATACTTCT | |
| | GTGCCAGCAGCCAGGACAGGTCCTATAATTCGCCCCTCTACTTTGCG | |
| | GCAGGCACCCGGCTCACTGTGACAGAGGATCTGAGAAATGTGACTC | |
| | CACCCAAGGTGAGCCTGTTTGAACCGAGCAAAGCGGAAATTGCGAA | |
| | CAAACAGAAAGCGACCCTGGTGTGCCTGGCGCGCGGCTTTTTTCCGG | |
| | ATCATGTGGAACTGAGCTGGTGGGTGAACGGCAAAGAAGTGCATAG | |
| | CGGCGTGAGCACCGATCCGCAGGCGTATAAAGAAAGCAACTATAGC | |
| | TATTGCCTGAGCAGCCGCCTGCGCGTGAGCGCGACCTTTTGGCATAA | |
| | CCCGCGCAACCATTTTCGCTGCCAGGTGCAGTTTCATGGCCTGAGCG | |
| | AAGAAGATAAATGGCCGGAAGGCAGCCCGAAACCGGTGACCCAGA | |
| | ACATTAGCGCGGAAGCGTGGGGCCGCGCGGATTGCGGCATTACCAG | |
| | CGCGAGCTATCAGCAGGGCGTGCTGAGCGCGACCATTCTGTATGAA | |
| | ATTCTGCTGGGCAAAGCGACCCTGTATGCGGTGCTGGTGAGCACCCT | |
| | GGTGGTGATGGCGATGGTGAAACGCAAAAACAGC | |
| 4 | MGSIFLSCLAVCLLVAGPVDPKIIQKPKYLVAVTGSEKILICEQYLGHNA | TCR beta chain, |
| | MYWYRQSAKKPLEFMFSYSYQKLMDNQTASSRFQPQSSKKNHLDLQIT | amino acid |
| | ALKPDDSATYFCASSQDRSYNSPLYFAAGTRLTVTEDLRNVTPPKVSLF | |
| | EPSKAEIANKQKATLVCLARGFFPDHVELSWWVNGKEVHSGVSTDPQA | |
| | YKESNYSYCLSSRLRVSATFWHNPRNHFRCQVQFHGLSEEDKWPEGSP | |
| | KPVTQNISAEAWGRADCGITSASYQQGVLSATILYEILLGKATLYAVLV | |
| | STLVVMAMVKRKNS | |
| 5 | MDKILTASFLLLGLHLAGVSGQQEKRDQQQVRQSPQSLTVWEGETAIL | TCR alpha chain |
| | NCSYENSAFDYFPWYQQFPGEGPALLISILSVSNKKEDGRFTIFFNKREK | variable region, |
| | KLSLHIADSQPGDSATYFCAGG | amino acid |
| 6 | DYANKMIFGLGTILRVRPH | TCR alpha chain |
| | | junction region, |
| | | amino acid |
| 7 | IQNPEPAVYQLKDPRSQDSTLCLFTDFDSQINVPKTMESGTFITDKTVLD | TCR alpha chain |
| | MKAMDSKSNGAIAWSNQTSFTCQDIFKETNATYPSSDVPCDATLTEKSF | constant region, |
| | ETDMNLNFQNLSVMGLRILLLKVAGFNLLMTLRLWSS | amino acid |
| 8 | MGSIFLSCLAVCLLVAGPVDPKIIQKPKYLVAVTGSEKILICEQYLGHNA | TCR beta chain |
| | MYWYRQSAKKPLEFMFSYSYQKLMDNQTASSRFQPQSSKKNHLDLQIT | variable region, |
| | ALKPDDSATYFCASSQ | amino acid |
| 9 | DRS | TCR beta chain |
| | | diversity region, |
| | | amino acid |
| 10 | YNSPLYFAAGTRLTVT | TCR beta chain |
| | | junction region, |
| | | amino acid |
| 11 | EDLRNVTPPKVSLFEPSKAEIANKQKATLVCLARGFFPDHVELSWWVN | TCR beta |
| | GKEVHSGVSTDPQAYKESNYSYCLSSRLRVSATFWHNPRNHFRCQVQF | constant region, |
| | HGLSEEDKWPEGSPKPVTQNISAEAWGRADCGITSASYQQGVLSATILY | amino acid |
| | EILLGKATLYAVLVSTLVVMAMVKRKNS | |
| 12 | NSAFDY | TCR alpha chain |
| | | CDR-1 |
| 13 | ILSVSNK | TCR alpha chain |
| | | CDR-2 |
| 14 | AGG | TCR alpha chain |
| | | CDR-3 |
| 15 | LGHNA | TCR beta chain |
| | | CDR-1 |
| 16 | YSYQKL | TCR beta chain |
| | | CDR-2 |
| 17 | ASSQ | TCR beta chain |
| | | CDR-3 |
| 18 | GTGCCGAAAACCATGGAATC TGG | TCR alpha |
| | | (TRAC) targeting |
| | | guide RNA |
| | | (mouse) |
| 19 | AATCAATGTGCCGAAAACCA TGG | TCR alpha |
| | | (TRAC) targeting |
| | | guide RNA |
| | | (mouse) |
| 20 | GCAGTTCCATGGGCTTTCAGAGG | TCR beta |
| | | (TRBC) targeting |
| | | guide RNA |
| | | (mouse) |
| 21 | TGGGGTCAGCACGGACCCTCAGG | TCR beta |
| | | (TRBC) targeting |
| | | guide RNA |
| | | (mouse) |
| 22 | TGTGCTAGACATGAGGTCTATGG | TCR alpha |
| | | (TRAC) targeting |
| | | guide RNA |
| | | (human) |
| 23 | GGAGAATGACGAGTGGACCCAGG | TCR beta |
| | | (TRBC) targeting |
| | | guide RNA |
| | | (human) |

What is claimed is:

1. An engineered cell, comprising a heterologous T cell receptor (TCR) or antigen-binding fragment thereof,
wherein the heterologous TCR or antigen-binding fragment thereof comprises an alpha chain comprising a variable alpha (Vα) region and a beta chain comprising a variable beta (Vβ) region, and
wherein the Vα region comprises a complementarity determining region 1 (CDR-1), CDR-2, and CDR-3 comprising the amino acid sequences of SEQ ID NOs: 12, 13, and 14, respectively; and the Vβ region comprises a CDR-1, CDR-2, and CDR-3 comprising the amino acid sequences of SEQ ID NOs: 15, 16, and 17, respectively.

2. An engineered cell, comprising a heterologous T cell receptor (TCR) or antigen-binding fragment thereof,
wherein the heterologous TCR or antigen-binding fragment thereof comprises an alpha chain comprising a variable alpha (Vα) region and a beta chain comprising a variable beta (Vβ) region, and
wherein the Vα region comprises a complementarity determining region 1 (CDR-1), CDR-2, and CDR-3 comprising the CDR-1, CDR-2, and CDR-3 amino acid sequences contained within the amino acid sequence of SEQ ID NO: 2 or 5; and the Vβ region comprises a CDR-1, CDR-2, and CDR-3 comprising the CDR-1, CDR-2, and CDR-3 amino acid sequences contained within the amino acid sequence of SEQ ID NO: 4 or 8.

3. The engineered cell of claim 1 or claim 2, further comprising a genetic disruption in one or more genes endogenous to the cell.

4. The engineered cell of claim 3, wherein the one or more genes endogenous to the cell comprises a T Cell Receptor Alpha Constant (TRAC) gene and/or a T Cell Receptor Beta Constant (TRBC) gene.

5. The engineered cell of any one of claims 1-4, wherein the engineered cell is a T cell, a B cell, or a Natural Killer (NK) cell.

6. The engineered cell of any one of claims 1-5, wherein the engineered cell is a T cell.

7. The engineered cell of claim 6, wherein the T cell is a regulatory T cell or an effector T cell.

8. The method of claim 6 or claim 7, wherein the T cell is a regulatory T (Treg) cell.

9. The engineered cell of any one of claims 1-8, further comprising a nucleic acid molecule encoding the TCR or antigen-binding fragment thereof.

10. The engineered cell of claim 9, wherein the nucleic acid molecule is under the control of one or more endogenous gene promoters.

11. The engineered cell of claim 10, wherein the one or more endogenous gene promoters comprises the endogenous TRAC gene promoter and/or the endogenous TRBC gene promoter.

12. The engineered cell of claim 9 or claim 11, wherein the nucleic acid molecule is under the control of an exogenous promoter.

13. The engineered cell of any one of claims 9-12, wherein the nucleic acid molecule is inserted into the endogenous TRAC gene and/or the endogenous TRBC gene.

14. An engineered cell, comprising:
a heterologous T cell receptor (TCR) or antigen-binding fragment thereof, wherein the heterologous TCR or antigen-binding fragment thereof comprises an alpha chain comprising a variable alpha (Vα) region and a beta chain comprising a variable beta (Vβ) region, and wherein the Vα region comprises a complementarity determining region 1 (CDR-1), CDR-2, and CDR-3 comprising the amino acid sequences of SEQ ID NOs: 12, 13, and 14, respectively; and the Vβ region comprises a CDR-1, CDR-2, and CDR-3 comprising the amino acid sequences of SEQ ID NOs: 15, 16, and 17, respectively; and
a genetic disruption in a T Cell Receptor Alpha Constant (TRAC) gene and/or a T Cell Receptor Beta Constant (TRBC) gene.

15. An engineered cell, comprising:
a heterologous T cell receptor (TCR) or antigen-binding fragment thereof, wherein the heterologous TCR or antigen-binding fragment thereof comprises an alpha chain comprising a variable alpha (Vα) region and a beta chain comprising a variable beta (Vβ) region, and wherein the Vα region comprises a complementarity determining region 1 (CDR-1), CDR-2, and CDR-3 comprising the CDR-1, CDR-2, and CDR-3 amino acid sequences contained within the amino acid sequence of SEQ ID NO: 2 or 5; and the Vβ region comprises a CDR-1, CDR-2, and CDR-3 comprising the CDR-1, CDR-2, and CDR-3 amino acid sequences contained within the amino acid sequence of SEQ ID NO: 4 or 8; and
a genetic disruption in a T Cell Receptor Alpha Constant (TRAC) gene and/or a T Cell Receptor Beta Constant (TRBC) gene.

16. An engineered regulatory T (Treg) cell, comprising:
a heterologous T cell receptor (TCR) or antigen-binding fragment thereof, wherein the heterologous TCR or antigen-binding fragment thereof comprises an alpha chain comprising a variable alpha (Vα) region and a beta chain comprising a variable beta (Vβ) region, and wherein the Vα region comprises a complementarity determining region 1 (CDR-1), CDR-2, and CDR-3 comprising the amino acid sequences of SEQ ID NOs: 12, 13, and 14, respectively; and the Vβ region comprises a CDR-1, CDR-2, and CDR-3 comprising the amino acid sequences of SEQ ID NOs: 15, 16, and 17, respectively; and
a genetic disruption in a T Cell Receptor Alpha Constant (TRAC) gene and/or a T Cell Receptor Beta Constant (TRBC) gene.

17. An engineered regulatory T (Treg) cell, comprising:
a heterologous T cell receptor (TCR) or antigen-binding fragment thereof, wherein the heterologous TCR or antigen-binding fragment thereof comprises an alpha chain comprising a variable alpha (Vα) region and a beta chain comprising a variable beta (Vβ) region, and wherein the Vα region comprises a complementarity determining region 1 (CDR-1), CDR-2, and CDR-3 comprising the CDR-1, CDR-2, and CDR-3 amino acid sequences contained within the amino acid sequence of SEQ ID NO: 2 or 5; and the Vβ region comprises a CDR-1, CDR-2, and CDR-3 comprising the CDR-1, CDR-2, and CDR-3 amino acid sequences contained within the amino acid sequence of SEQ ID NO: 4 or 8; and
a genetic disruption in a T Cell Receptor Alpha Constant (TRAC) gene and/or a T Cell Receptor Beta Constant (TRBC) gene.

18. An engineered cell, comprising:
a nucleic molecule encoding a heterologous T cell receptor (TCR) or antigen-binding fragment thereof, wherein the heterologous TCR or antigen-binding fragment thereof comprises an alpha chain comprising a variable alpha (Vα) region and a beta chain comprising a variable beta (Vβ) region, and wherein the Vα region comprises a complementarity determining region 1 (CDR-1), CDR-2, and CDR-3 comprising the amino acid sequences of SEQ ID NOs: 12, 13, and 14, respectively; and the Vβ region comprises a CDR-1, CDR-2, and CDR-3 comprising the amino acid sequences of SEQ ID NOs: 15, 16, and 17, respectively; and
a genetic disruption in a T Cell Receptor Alpha Constant (TRAC) gene and/or a T Cell Receptor Beta Constant (TRBC) gene.

19. An engineered cell, comprising:
a nucleic acid molecule encoding a heterologous T cell receptor (TCR) or antigen-binding fragment thereof, wherein the heterologous TCR or antigen-binding fragment thereof comprises an alpha chain comprising a variable alpha (Vα) region and a beta chain comprising a variable beta (Vβ) region, and wherein the Vα region comprises a complementarity determining region 1 (CDR-1), CDR-2, and CDR-3 comprising the CDR-1, CDR-2, and CDR-3 amino acid sequences contained within the amino acid sequence of SEQ ID NO: 2 or 5; and the Vβ region comprises a CDR-1, CDR-2, and CDR-3 comprising the CDR-1, CDR-2, and CDR-3 amino acid sequences contained within the amino acid sequence of SEQ ID NO: 4 or 8; and
a genetic disruption in a T Cell Receptor Alpha Constant (TRAC) gene and/or a T Cell Receptor Beta Constant (TRBC) gene.

20. The engineered cell of any one of claims 15-17, further comprising a nucleic acid molecule encoding the TCR or antigen-binding fragment thereof.

21. The engineered cell of any one of claims 18-20, wherein the nucleic acid molecule is under the control of one or more endogenous gene promoters.

22. The engineered cell of claim 21, wherein the one or more endogenous gene promoters comprises the endogenous TRAC gene promoter and/or the endogenous TRBC gene promoter.

23. The engineered cell of any one of claims 18-22, wherein the nucleic acid molecule is under the control of an exogenous promoter.

24. The engineered cell of any one of claims 18-23, wherein the nucleic acid molecule is inserted into the endogenous TRAC gene and/or the endogenous TRBC gene.

25. The engineered cell of any one of claims 9-11, 13, 18-22, and 24, wherein the nucleic acid molecule is under the control of the endogenous TRAC gene promoter and/or the endogenous TRBC gene promoter.

26. The engineered cell of any one of claims 3-25, wherein the genetic disruption results in a reduction or elimination of expression of the gene product of the endogenous gene.

27. The engineered cell of any one of claims 1-26, wherein:
the Vα region comprises a CDR-1, CDR-2, and CDR-3 comprising the amino acid sequences of SEQ ID NOs: 12, 13, and 14, respectively; and the Vβ region comprises a CDR-1, CDR-2, and CDR-3 comprising the amino acid sequences of SEQ ID NOs: 15, 16, and 17, respectively; or
the Vα region comprises a CDR-1, CDR-2, and CDR-3 comprising the CDR-1, CDR-2, and CDR-3 amino acid sequences contained within the amino acid sequence of SEQ ID NO: 2 or 5; and the Vβ region comprises a CDR-1, CDR-2, and CDR-3 comprising the CDR-1, CDR-2, and CDR-3 amino acid sequences contained within the amino acid sequence of SEQ ID NO: 4 or 8.

28. The engineered cell of any one of claims 1-27, wherein the Vα region comprises the amino acid sequence of SEQ ID NO: 5 or an amino acid sequence having at least 80%, 85%, 90%, 91%, 92%, 93%, 94%, 95%, 96%, 97%, 98%, or 99% identity to SEQ ID NO: 5; and/or the Vβ comprises the amino acid sequence of SEQ ID NO: 8 or an amino acid sequence having at least 80%, 85%, 90%, 91%, 92%, 93%, 94%, 95%, 96%, 97%, 98%, or 99% identity to the amino acid sequence of SEQ ID NO: 8.

29. The engineered cell of any one of claims 1-28, wherein the Vα region comprises an amino acid sequence of SEQ ID NO: 5 and/or the Vβ region comprises the amino acid sequence of SEQ ID NO: 8.

30. The engineered cell of any one of claims 1-29, wherein the Vα region comprises an amino acid sequence of SEQ ID NO: 5 and the Vβ region comprises the amino acid sequence of SEQ ID NO: 8.

31. The engineered cell of any one of claims 1-30, wherein the TCR or antigen-binding fragment thereof specifically binds to human amyloid beta.

32. The engineered cell of any one of claims 1-31, wherein the alpha chain further comprises an alpha constant (Cα) region and/or the beta chain further comprises a beta constant (Cβ) region.

33. The engineered cell of claim 32, wherein the Cα region comprises the amino acid sequence of SEQ ID NO: 7, or an amino acid sequence having at least 80%, 85%, 86%, 87%, 88%, 89%, 90%, 91%, 92%, 93%, 94%, 95%, 96%, 97%, 98%, or 99% sequence identity to the amino acid sequence of SEQ ID NO: 7; and/or the Cβ region comprises the amino acid sequence of SEQ ID NO: 11, or an amino acid sequence having at least 80%, 85%, 86%, 87%, 88%, 89%, 90%, 91%, 92%, 93%, 94%, 95%, 96%, 97%, 98%, or 99% sequence identity to the amino acid sequence of SEQ ID NO: 11.

34. The engineered cell of claim 32, wherein the Cα region comprises the amino acid sequence of SEQ ID NO: 7, or an amino acid sequence having at least 80%, 85%, 86%, 87%, 88%, 89%, 90%, 91%, 92%, 93%, 94%, 95%, 96%, 97%, 98%, or 99% sequence identity to the amino acid sequence of SEQ ID NO: 7; and the Cβ region comprises the amino acid sequence of SEQ ID NO: 11, or an amino acid sequence having at least 80%, 85%, 86%, 87%, 88%, 89%, 90%, 91%, 92%, 93%, 94%, 95%, 96%, 97%, 98%, or 99% sequence identity to the amino acid sequence of SEQ ID NO: 11.

35. The engineered cell of claim 32, wherein the Cα region comprises the amino acid sequence of SEQ ID NO: 7; and/or the Cβ region comprises the amino acid sequence of SEQ ID NO: 11.

36. The engineered cell of any one of claims 1-35, wherein the alpha chain comprises the amino acid sequence of SEQ ID NO: 2 or an amino acid sequence having at least 80%, 85%, 90%, or 95% identity to SEQ ID NO: 2; and/or the beta chain comprises the amino acid sequence of SEQ ID NO: 4 or an amino acid sequence having at least 80%, 85%, 90%, or 95% identity to SEQ ID NO: 4.

37. The engineered cell of any one of claims 1-36, that is chimeric.

38. The engineered cell of any one of claims 1-28 and 31-36, that is humanized.

39. The engineered cell of any one of claims 1-36, that is murine.

40. The engineered cell of any one of claims 14, 15, and 18-39, wherein the engineered cell is a T cell, a B cell, or a Natural Killer (NK) cell.

41. The engineered cell of any one of claims 14, 15, and 18-40, wherein the engineered cell is a T cell.

42. The engineered cell of claim 41, wherein the T cell is a regulatory T cell or an effector T cell.

43. The engineered cell of claim 41 or claim 42, wherein the T cell is a regulatory T (Treg) cell.

44. A T cell receptor (TCR) or antigen-binding fragment thereof, comprising an alpha chain comprising a variable alpha (Vα) region and a beta chain comprising a variable beta (Vβ) region, wherein the Vα region comprises a complementarity determining region 1 (CDR-1), CDR-2, and CDR-3 comprising the amino acid sequences of SEQ ID NOs: 12, 13, and 14, respectively; and the Vβ region comprises a CDR-1, CDR-2, and CDR-3 comprising the amino acid sequences of SEQ ID NOs: 15, 16, and 17, respectively.

45. A T cell receptor (TCR) or antigen-binding fragment thereof, comprising an alpha chain comprising a variable alpha (Vα) region and a beta chain comprising a variable beta (Vβ) region, wherein the Vα region comprises a complementarity determining region 1 (CDR-1), CDR-2, and CDR-3 comprising the CDR-1, CDR-2, and CDR-3 amino acid sequences contained within the amino acid sequence of SEQ ID NO: 2 or 5; and the Vβ region comprises a CDR-1, CDR-2, and CDR-3 comprising the CDR-1, CDR-2, and CDR-3 amino acid sequences contained within the amino acid sequence of SEQ ID NO: 4 or 8.

46. The TCR or antigen-binding fragment thereof of claim 37 or claim 38, wherein the Vα region comprises a CDR-1, CDR-2, and CDR-3 comprising the CDR-1, CDR-2, and CDR-3 amino acid sequences contained within the amino acid sequence of SEQ ID NO: 2; and the Vβ region comprises a CDR-1, CDR-2, and CDR-3 comprising the CDR-1, CDR-2, and CDR-3 amino acid sequences contained within the amino acid sequence of SEQ ID NO: 4.

47. The TCR or antigen-binding fragment thereof of claim 44 or claim 45, wherein the Vα region comprises a CDR-1, CDR-2, and CDR-3 comprising the CDR-1, CDR-2, and CDR-3 amino acid sequences contained within the amino acid sequence of SEQ ID NO: 5; and the Vβ region comprises a CDR-1, CDR-2, and CDR-3 comprising the CDR-1, CDR-2, and CDR-3 amino acid sequences contained within the amino acid sequence of SEQ ID NO: 8.

48. The TCR or antigen-binding fragment thereof of any one of claims 44-47, wherein the Vα region comprises the amino acid sequence of SEQ ID NO: 5 or an amino acid sequence having at least 80%, 85%, 90%, 91%, 92%, 93%, 94%, 95%, 96%, 97%, 98%, or 99% identity to SEQ ID NO: 5; and/or wherein the Vβ comprises the amino acid sequence of SEQ ID NO: 8 or an amino acid sequence having at least 80%, 85%, 90%, 91%, 92%, 93%, 94%, 95%, 96%, 97%, 98%, or 99% identity to the amino acid sequence of SEQ ID NO: 8.

49. The TCR or antigen-binding fragment thereof of any one of claims 44-48, wherein the Vα region comprises an amino acid sequence of SEQ ID NO: 5 and/or the Vβ region comprises the amino acid sequence of SEQ ID NO: 8.

50. The TCR or antigen-binding fragment thereof of any one of claims 44-49, wherein the Vα region comprises an amino acid sequence of SEQ ID NO: 5 and the Vβ region comprises the amino acid sequence of SEQ ID NO: 8.

51. The TCR or antigen-binding fragment thereof of any one of claims 44-50, wherein the TCR or antigen-binding fragment thereof specifically binds to amyloid beta.

52. The TCR or antigen-binding fragment thereof of any one of claims 44-51, wherein the TCR or antigen-binding fragment thereof specifically binds to human amyloid beta.

53. The TCR or antigen-binding fragment thereof of any one of claims 44-52, wherein the alpha chain further comprises an alpha constant (Cα) region and/or the beta chain further comprises a beta constant (Cβ) region.

54. The TCR or antigen-binding fragment thereof of claim 53, wherein the Cα region comprises the amino acid sequence of SEQ ID NO: 7, or an amino acid sequence having at least 80%, 85%, 86%, 87%, 88%, 89%, 90%, 91%, 92%, 93%, 94%, 95%, 96%, 97%, 98%, or 99% sequence identity to the amino acid sequence of SEQ ID NO: 7; and/or the Cβ region comprises the amino acid sequence of SEQ ID NO: 11, or an amino acid sequence having at least 80%, 85%, 86%, 87%, 88%, 89%, 90%, 91%, 92%, 93%, 94%, 95%, 96%, 97%, 98%, or 99% sequence identity to the amino acid sequence of SEQ ID NO: 11.

55. The TCR or antigen-binding fragment thereof of claim 53, wherein the Cα region comprises the amino acid sequence of SEQ ID NO: 7, or an amino acid sequence having at least 80%, 85%, 86%, 87%, 88%, 89%, 90%, 91%, 92%, 93%, 94%, 95%, 96%, 97%, 98%, or 99% sequence identity to the amino acid sequence of SEQ ID NO: 7; and the Cβ region comprises the amino acid sequence of SEQ ID NO: 11, or an amino acid sequence having at least 80%, 85%, 86%, 87%, 88%, 89%, 90%, 91%, 92%, 93%, 94%, 95%, 96%, 97%, 98%, or 99% sequence identity to the amino acid sequence of SEQ ID NO: 11.

56. The TCR or antigen-binding fragment thereof of claim 53, wherein the Cα region comprises the amino acid sequence of SEQ ID NO: 7; and/or the Cβ region comprises the amino acid sequence of SEQ ID NO: 11.

57. The TCR or antigen-binding fragment thereof of any one of claims 44-56, wherein the alpha chain comprises the amino acid sequence of SEQ ID NO: 2 or an amino acid sequence having at least 80%, 85%, 90%, or 95% identity to SEQ ID NO: 2; and/or the beta chain comprises the amino acid sequence of SEQ ID NO: 4 or an amino acid sequence having at least 80%, 85%, 90%, or 95% identity to SEQ ID NO: 4.

58. The TCR or antigen-binding fragment thereof of any one of claims 44-57, that is chimeric.

59. The TCR or antigen-binding fragment thereof of any one of claims 44-48, 51-55, and 57, that is humanized.

60. A nucleic acid molecule encoding the TCR or antigen-binding fragment thereof of any one of claims 44-59, or an alpha or beta chain thereof, or a Vα region or Vβ region thereof.

61. The nucleic acid molecule of claim 60, that is isolated.

62. The nucleic acid molecule of claim 60 or claim 61, wherein the nucleic acid molecule is codon-optimized.

63. The nucleic acid molecule of claim 60 or claim 61, wherein the nucleic acid molecule is not codon-optimized.

64. The nucleic acid molecule of any one of claims 60-63, wherein the nucleic acid molecule is DNA.

65. The nucleic acid molecule of claim 64, wherein the DNA is cDNA.

66. The nucleic acid molecule of any one of claims 60-63, wherein the nucleic acid molecule is RNA.

67. The nucleic acid molecule of any one of claims 60-66, wherein the TCR or antigen-binding fragment thereof that is encoded is humanized or chimeric.

68. The nucleic acid molecule of any one of claims 60-66, wherein the TCR or antigen-binding fragment thereof that is encoded is murine.

69. The nucleic acid molecule of any one of claims 60-68, comprising the nucleic acid sequence of SEQ ID NO: 1, or a nucleic acid sequence having at least 80%, 85%, 90%, 91%, 92%, 93%, 94%, 95%, 96%, 97%, 98%, or 99% sequence identity to the nucleic acid sequence of SEQ ID NO: 1; and/or the nucleic acid sequence of SEQ ID NO: 3, or a nucleic acid sequence having at least 80%, 85%, 90%, 91%, 92%, 93%, 94%, 95%, 96%, 97%, 98%, or 99% sequence identity to the nucleic acid sequence of SEQ ID NO: 3.

70. A vector comprising the nucleic acid molecule of any one of claims 60-69.

71. A vector comprising a nucleic acid molecule encoding the TCR or antigen-binding fragment thereof of any one of claims 44-59, or an alpha and/or beta chain thereof, or a Vα region and/or Vβ region thereof.

72. The vector of claim 70 or claim 71, wherein the vector is an expression vector.

73. The vector of any one of claims 70-72, wherein the vector is a viral vector.

74. The vector of claim 73, wherein the viral vector is selected from the group consisting of a retroviral vector, a gammaretroviral vector, a lentiviral vector, and an adeno-associated viral (AAV) vector.

75. The vector of claim 74, wherein the AAV vector is a self-complementary AAV (scAAV) vector.

76. The vector of any one of claims 70-72, wherein the vector is a non-viral vector.

77. The vector of any one of claims 70-72, wherein the vector is a donor vector for genome editing.

78. The vector of any one of claims 70-72, wherein the vector is a transposon vector.

79. The vector of claim 78, wherein the transposon vector is a Sleeping Beauty transposon vector or a PiggyBac transposon vector.

80. The vector of any one of claims 70-79, wherein the vector is suitable for gene editing or genomic engineering.

81. An engineered cell comprising the nucleic acid molecule of any one of claims 60-69 or the vector of any one of claims 70-80.

82. An engineered cell comprising the TCR or antigen-binding fragment thereof of any one of claims 44-59.

83. The engineered cell of claim 82, wherein the TCR or antigen-binding fragment thereof is heterologous to the engineered cell.

84. A method of producing an engineered cell, comprising introducing a nucleic acid molecule of any one of claims 60-69 or a vector of any one of claims 70-80, into a cell.

85. A method of producing the engineered cell of any one of claims 1-43, comprising introducing a nucleic acid molecule of any one of claims 60-69 or a vector of any one of claims 70-80, into a cell.

86. The method of claim 84 or claim 85, that is performed in vitro or ex vivo.

87. The method of any one of claims 84-86, wherein the nucleic acid molecule is comprised within a vector.

88. A method of producing a population of engineered cells, comprising introducing a nucleic acid molecule of any one of claims 60-69 or a vector of any one of claims 70-80, into a cell; and culturing the cell under conditions to produce a population of engineered cells.

89. A method of producing a population of engineered cells, comprising culturing the engineered cell of any one of claims 1-43 under conditions to produce a population of engineered cells.

90. A method of engineering a cell, comprising introducing a nucleic acid molecule of any one of claims 60-69 or a vector of any one of claims 70-80, into a cell.

91. A method of engineering a cell, comprising:
introducing a nucleic acid molecule of any one of claims 60-69 or a vector of any one of claims 70-80, into a cell; and
editing and/or disrupting one or more genes endogenous to the cell.

92. The method of any one of claims 84-88, 90, and 91, wherein the introducing is carried out by transfection, electroporation, or transduction.

93. The method of any one of claims 84-88, 90, and 91, wherein the introducing is carried out by transfection.

94. The method of claim 93, wherein the introducing by transfection comprises introducing a vector of any one of claims 70-80 into the cell.

95. The method of any one of claims 84-88, 90, and 91, wherein the introducing is carried out by electroporation.

96. The method of any one of claims 83-88, 90, and 91, wherein the introducing is carried out by transduction.

97. The method of claim 96, wherein the introducing by transduction comprises introducing a vector of any one of claims 70-80 into the cell.

98. The method of any one of claims 84-88 and 90-97, wherein the vector is a viral vector.

99. The method of claim 98, wherein the viral vector is selected from the group consisting of a retroviral vector, a gammaretroviral vector, a lentiviral vector, and an adeno-associated viral (AAV) vector.

100. The method of claim 99, wherein the AAV vector is a self-complementary AAV (scAAV) vector.

101. The method of any one of claims 84-88 and 90-100, wherein the introducing is carried out using a genome editing technique.

102. The method of any one of claims 84-88 and 90-100, the introducing is carried out using a genome editing technique; and/or the method further comprises a genome editing technique.

103. The method of claim 101 or claim 102, wherein the genome editing technique results in editing and/or disrupting one or more genes endogenous to the cell; and/or introducing the nucleic acid molecule encoding the TCR or antigen-binding fragment thereof into a target site.

104. The method of claim 103, wherein the target site is comprised within one or more genes endogenous to the cell.

105. The method of claim 104, wherein the one or more genes endogenous to the cell comprises a T Cell Receptor Alpha Constant (TRAC) gene and/or a T Cell Receptor Beta Constant (TRBC) gene.

106. The method of any one of claims 101-105, wherein the genome editing technique comprises CRISPR-Cas9 and comprises introducing a crRNA sequence targeting a human TRAC gene that comprises a nucleic acid sequence set forth in SEQ ID NO: 22, and/or comprises introducing a crRNA sequence targeting a human TRBC gene that comprises a nucleic acid sequence set forth in SEQ ID NO: 23.

107. The method of any one of claims 84-106, further comprising introducing into the cell one or more agents capable of editing and/or disrupting one or more genes endogenous to the cell.

108. The method of any one of claim 107, wherein the editing and/or disrupting is carried out by one or more agents capable of editing and/or disrupting the one or more genes endogenous to the cell.

109. The method of claim 107 or claim 108, wherein the editing and/or disrupting reduces or eliminates expression of the one or more genes endogenous to the cell.

110. The method of any one of claims 107-109, wherein the editing and/or disrupting eliminates expression of the one or more genes endogenous to the cell.

111. The method of any one of claims 107-110, wherein the one or more genes endogenous to the cell each comprise a target site, and one or more of the one or more agents specifically bind to or recognizes the target site.

112. The method of any one of claims 107-111, wherein the one or more genes endogenous to the cell comprises a T Cell Receptor Alpha Constant (TRAC) gene and/or a T Cell Receptor Beta Constant (TRBC) gene.

113. The method of any one of claims 107-112, wherein the one or more agents comprises a nuclease.

114. The method of claim 113, wherein the nuclease specifically binds to or recognizes the target site.

115. The method of claim 113 or claim 114, wherein the nuclease is selected from the group consisting of a meganuclease, a zinc-finger nuclease, a transcription activator-like effector nuclease (TALEN), a megaTAL nuclease, and a clustered regularly interspaced short palindromic repeats (CRISPR)-associated (Cas) nuclease.

116. The method of any one of claims 113-115, wherein the nuclease is a Cas nuclease.

117. The method of claim 116, wherein the Cas nuclease is a Cas9, Cas12a, or Cas13 nuclease.

118. The method of any one of claims 113-117, wherein the Cas nuclease is a Cas9 nuclease.

119. The method of any one of claims 84-118, further comprising introducing into the cell one or more agents capable of inserting the nucleic acid molecule into the genome of the cell.

120. The method of claim 119, wherein the one or more agents capable of inserting the nucleic acid molecule into the genome of the cell comprises a transposon or a transposon-based system.

121. The method of claim 120, wherein the transposon comprises a Sleeping Beauty transposon or a PiggyBac transposon; or the transposon-based system comprises a Sleeping Beauty transposon-based system or a PiggyBac transposon-based system.

122. A method of engineering a cell, comprising:
introducing, into the cell, one or more agents capable of editing and/or disrupting one or more endogenous genes in the cell, wherein each of the one or more endogenous genes comprises a first flanking sequence and a second flanking sequence; and
introducing, into the cell, one or more nucleic acid molecules, wherein each of the one or more nucleic acid molecules comprises: (i) a first homology arm and a second homology arm that are homologous to the first flanking sequence and the second flanking sequence of one of the one or more endogenous genes, and (ii) a nucleic acid sequence of interest that is located between the first homology arm and the second homology arm.

123. A method of engineering a cell, comprising:
introducing, into the cell, one or more agents capable of inducing a DNA break in one or more endogenous genes in the cell, wherein each of the one or more endogenous genes comprises a first flanking sequence and a second flanking sequence; and
introducing, into the cell, one or more nucleic acid molecules, wherein each of the one or more nucleic acid molecules comprises: (i) a first homology arm and a second homology arm that are homologous to the first flanking sequence and the second flanking sequence of one of the one or more endogenous genes, and (ii) a nucleic acid sequence of interest that is located between the first homology arm and the second homology arm.

124. The method of claim 122 or claim 123, wherein the nucleic acid sequence of interest encodes a T cell receptor (TCR) or antigen-binding fragment thereof, wherein the TCR or antigen-binding fragment thereof comprises an alpha chain comprising a variable alpha (Vα) region and a beta chain comprising a variable beta (Vβ) region, wherein the Vα region comprises a complementarity determining region 1 (CDR-1), CDR-2, and CDR-3 comprising the amino acid sequences of SEQ ID NOs: 12, 13, and 14, respectively; and the Vβ region comprises a CDR-1, CDR-2, and CDR-3 comprising the amino acid sequences of SEQ ID NOs: 15, 16, and 17, respectively.

125. The method of any one of claims 122-124, wherein the nucleic acid sequence of interest comprises the nucleic acid molecule of any one of claims 60-69.

126. The method of any one of claims 122-125, wherein the one or more endogenous genes comprises a T Cell Receptor Alpha Constant (TRAC) gene and/or a T Cell Receptor Beta Constant (TRBC) gene.

127. The method of any one of claims 122 and 124-126, wherein the one or more agents capable of editing and/or disrupting one or more endogenous genes in the cell comprises a nuclease.

128. The method of any one of claims 123-126, wherein the one or more agents capable of inducing a DNA break comprises a nuclease.

129. The method of claim 127 or claim 128, wherein the nuclease is selected from the group consisting of a meganuclease, a zinc-finger nuclease, a transcription activator-like effector nuclease (TALEN), a megaTAL nuclease, and a clustered regularly interspaced short palindromic repeats (CRISPR)-associated (Cas) nuclease.

130. The method of any one of claims 127-129, wherein the nuclease is a Cas nuclease.

131. The method of claim 130, wherein the Cas nuclease is a Cas9, Cas12a, or Cas13 nuclease.

132. The method of claim 130 or claim 131, wherein the Cas nuclease is a Cas9 nuclease.

133. The method of any one of claims 122, 124-127, and 129-132, wherein the one or more agents capable of editing and/or disrupting one or more endogenous genes in the cell comprises a Cas nuclease and one or more single guide RNA (sgRNA).

134. The method of any one of claims 123-126 and 128-132, wherein the one or more agents capable of inducing a DNA break comprises a Cas nuclease and one or more single guide RNA (sgRNA).

135. The method of claim 133 or claim 134, wherein each of the one or more sgRNA specifically binds to, hybridizes with, or recognizes a target sequence in one of the one or more endogenous genes.

136. The method of any one of claims 133-135, wherein the one or more sgRNA comprises an sgRNA that specifically binds to, hybridizes with, or recognizes a target sequence in an endogenous TRAC gene, and/or comprises an sgRNA that specifically binds to, hybridizes with, or recognizes a target sequence in an endogenous TRBC gene.

137. The method of any one of claims 122-136, wherein the method results in reduced or eliminated expression of the one or more endogenous genes; and/or introduces expression of the TCR or antigen-binding fragment thereof in the cell

138. The method of any one of claims 122-137, wherein the method results in reduced or eliminated expression of the one or more endogenous genes and incorporation of a nucleic acid molecule encoding the TCR or antigen-binding fragment thereof into a target site contained within the one or more endogenous genes.

139. The method of any one of claims 122-138, wherein the one or more nucleic acid molecules is comprised within a vector.

140. The method of claim 139, wherein the vector is a donor vector.

141. The method of claim 139, wherein the vector is an expression vector.

142. The method of any one of claims 139-141, wherein the vector is a viral vector.

143. The method of claim 142, wherein the viral vector is selected from the group consisting of a retroviral vector, a gammaretroviral vector, a lentiviral vector, and an adeno-associated viral (AAV) vector.

144. The method of claim 143, wherein the AAV vector is a self-complementary AAV (scAAV) vector.

145. The method of any one of claims 139-141, wherein the vector is a non-viral vector.

146. The method of any one of claims 139-141, wherein the vector is a donor vector for genome editing.

147. The method of any one of claims 139-141, wherein the vector is a transposon vector.

148. The method of claim 147, wherein the transposon vector is a Sleeping Beauty transposon vector or a PiggyBac transposon vector.

149. The vector of any one of claims 139-148, wherein the vector is suitable for gene editing or genomic engineering.

150. The method of any one of claims 122-149, wherein the introducing of one or more of: (i) the one or more agents capable of editing and/or disrupting one or more endogenous genes in the cell, (ii) one or more agents capable of inducing a DNA break in one or more endogenous genes in the cell, and/or (iii) one or more nucleic acid molecules, is carried out by transfection, electroporation, or transduction, or any combination thereof.

151. The method of claim 150, wherein the introducing of one or more of: (i) the one or more agents capable of editing and/or disrupting one or more endogenous genes in the cell, (ii) one or more agents capable of inducing a DNA break in one or more endogenous genes in the cell, and/or (iii) one or more nucleic acid molecules, is carried out by transfection.

152. The method of claim 151, wherein the introducing of the one or more nucleic acid molecules by transfection comprises introducing a vector of any one of claims 70-80 into the cell.

153. The method of any one of claims 122-152, wherein the introducing one or more of: (i) the one or more agents capable of editing and/or disrupting one or more endogenous genes in the cell, (ii) one or more agents capable of inducing a DNA break in one or more endogenous genes in the cell, and/or (iii) one or more nucleic acid molecules, is carried out by electroporation.

154. The method of any one of claims 122-153, wherein the introducing one or more of: (i) the one or more agents capable of editing and/or disrupting one or more endogenous genes in the cell, (ii) one or more agents capable of inducing a DNA break in one or more endogenous genes in the cell, and/or (iii) one or more nucleic acid molecules, is carried out by transduction.

155. The method of claim 154, wherein the introducing the one or more nucleic acid molecules by transduction comprises introducing a vector of any one of claims 70-80 into the cell.

156. The method of any one of claims 122-155, wherein the introducing the one or more nucleic acid molecules occurs by a transfection method.

157. The method of claim 156, wherein the transfection method is electroporation.

158. The method of any one of claims 84-157, wherein the cell is from a subject having a disease or condition associated with amyloid beta.

159. The method of claim 158, wherein the disease or condition associated with amyloid beta is a disease or condition associated with human amyloid beta.

160. The method of claim 158, wherein the disease or condition associated with amyloid beta is Alzheimer's Disease.

161. The method of any one of claims 84-157, wherein the cell is from a subject having Alzheimer's Disease.

162. The method of any one of claims 84-157, wherein the cell is from a donor subject.

163. The method of claim 162, wherein the donor subject does not have a disease or condition associated with amyloid beta or has not been diagnosed as having a disease or condition associated with amyloid beta.

164. The method of claim 162 or claim 163, wherein the donor subject does not have Alzheimer's Disease or has not been diagnosed as having Alzheimer's Disease.

165. The method of any one of claims 84-164, wherein the cell is a T cell, a B cell, or a Natural Killer (NK) cell.

166. The method of claim 165, wherein the engineered cell is a T cell.

167. The method of claim 166, wherein the T cell is a regulatory T cell or an effector T cell.

168. The method of claim 166 or claim 167, wherein the T cell is a regulatory T cell.

169. The method of claim 166 or claim 167, wherein the T cell is a CD4+ and/or CD8+ T cell.

170. The method of any one of claims 166-168, wherein the T cell is a CD4+/CD25+/FOXP3+ T cell.

171. An engineered cell produced by the method of any one of claims 84-170.

172. The engineered cell of any one of claims 1-43 and 171, wherein the engineered cell is a regulatory T cell.

173. A conjugate, comprising the TCR or antigen-binding fragment thereof of any one of claims 44-59, and a heterologous moiety.

174. The conjugate of claim 173, wherein the heterologous moiety is a detectable label.

175. The conjugate of claim 174, wherein the detectable label is a fluorescent label.

176. The conjugate of claim 174, wherein the detectable label is a radioisotope, a fluorescent label, or an enzyme-substrate.

177. A composition, comprising the engineered cells of any one of claims 1-43 and 171, or the TCR or antigen-binding fragment thereof of any one of claims 44-59, or the conjugate of any one of claims 173-176.

178. The composition of claim 177, further comprising a pharmaceutically acceptable excipient.

179. A method of treatment of a disease or condition associated with amyloid beta, comprising administering the engineered cell of any one of claims 1-43 and 171, or the composition of claim 177 or claim 178, to a subject having a disease or condition associated with amyloid beta.

180. The method of claim 179, wherein the disease or condition associated with amyloid beta is Alzheimer's Disease.

181. A method of treatment of Alzheimer's Disease, comprising administering the engineered cell of any one of claims 1-43 and 171, or the composition of claim 177 or claim 178, to a subject having Alzheimer's Disease.

182. A method of treatment of a disease or condition associated with amyloid beta, comprising administering the composition of claim 177 or claim 178 to a subject having a disease or condition associated with amyloid beta.

183. The method of claim 182, wherein the disease or condition associated with amyloid beta is Alzheimer's Disease.

184. A method of treatment of a disease or condition associated with amyloid beta, comprising administering the composition of claim 177 or claim 178 to a subject having Alzheimer's Disease.

185. A method of treating a disease or condition associated with amyloid beta, comprising:
a) engineering a cell by the method of any one of claims 84-170;
b) culturing the engineered cell under conditions to produce a population of engineered cells; and
c) administering a therapeutically effective amount of the population of engineered cells to a subject having a disease or condition associated with amyloid beta.

186. A method of treating Alzheimer's Disease, comprising:
a) engineering a cell by the method of any one of claims 84-170;
b) culturing the engineered cell under conditions to produce a population of engineered cells; and
c) administering a therapeutically effective amount of the population of engineered cells to a subject having Alzheimer's Disease.

187. The method of any one of claims 179-186, wherein the subject is a human.

188. The method of any one of claims 179-187, wherein the engineered cells are autologous to the subject.

189. The method of any one of claims 179-187, wherein the engineered cells are allogenic to the subject.

190. The method of any one of claims 179-189, wherein the engineered cells are T cells, B cells, or Natural Killer (NK) cells.

191. The method of claim 190, wherein the engineered cells are T cells.

192. The method of claim 191, wherein the T cells are regulatory T cells or effector T cells.

193. The method of claim 191, wherein the T cells are regulatory T cells.

194. The method of claim 192 or claim 193, wherein the T cells are CD4+ and/or CD8+ T cells.

195. The method of claim 192 or claim 193, wherein the T cells are CD4+/CD25+/FOXP3+ T cells.

196. A composition of claim 177 or claim 178 for use in treating a disease or condition associated with amyloid beta in a subject.

197. Use of a composition of claim 177 or claim 178 for the manufacture of a medicament for treating a disease or condition associated with amyloid beta in a subject.

198. The composition for use of claim 196 or the use of claim 197, wherein the subject is a human.

199. The composition for use of claim 196 or claim 198, or the use of claim 197 or claim 198, wherein the disease or condition associated with amyloid beta is Alzheimer's Disease.

200. A method of diagnosing a disease or condition associated with amyloid beta, comprising administering the TCR or antigen-binding fragment thereof of any one of claims 44-59, or the conjugate of any one of claims 173-176, or the composition of claim 177 or claim 178, to a subject having or suspected of having a disease or condition associated with amyloid beta.

201. The method of claim 200, further comprising detecting the level or absence of binding between the TCR or antigen-binding fragment thereof or the conjugate with amyloid beta.

202. The method of claim 201, further comprising comparing the level or absence of binding to the level or absence of binding between the TCR or antigen-binding fragment thereof or the conjugate with amyloid beta as detected as one or more preceding time points.

203. A method of monitoring the progression of a disease or condition associated with amyloid beta, comprising administering the TCR or antigen-binding fragment thereof of any one of claims 44-59 or the conjugate of any one of claims 173-176, or the composition of claim 177 or claim 178, to a subject having a disease or condition associated with amyloid beta.

204. The method of claim 203, further comprising detecting the level or absence of binding between the TCR or antigen-binding fragment thereof or the conjugate with amyloid beta.

205. The method of claim 204, further comprising comparing the level or absence of binding to the level or absence of binding between the TCR or antigen-binding fragment thereof or the conjugate with amyloid beta as detected as one or more preceding time points.

206. A method of diagnosing Alzheimer's Disease, comprising administering the TCR or antigen-binding fragment thereof of any one of claims 44-59, or the conjugate of any one of claims 173-176, or the composition of claim 177 or claim 178, to a subject having or suspected of having Alzheimer's Disease.

207. A method of monitoring the progression of Alzheimer's Disease, comprising administering the TCR or antigen-binding fragment thereof of any one of claims 44-59, or the conjugate of any one of claims 173-176, or the composition of claim 177 or claim 178, to a subject having Alzheimer's Disease.

208. A composition of claim 177 or claim 178 for use in treating a disease or condition associated with amyloid beta in a subject.

209. Use of a composition of claim 177 or claim 178 for the manufacture of a medicament for treating a disease or condition associated with amyloid beta in a subject.

210. A composition of claim 177 or claim 178 for use in treating Alzheimer's Disease in a subject.

211. Use of a composition of claim 177 or claim 178 for the manufacture of a medicament for treating Alzheimer's Disease in a subject.

212. The composition for use of claim 208 or claim 210, or the use of claim 209 or claim 211, wherein the subject is a human.


SEQUENCE LISTING (23 sequences)

SEQ ID NO: 1 - DNA, 825 bp, synthetic construct
  misc_feature 1..825: note = TCR alpha chain, DNA
  source 1..825: mol_type = other DNA
atggacaagatcctgacagcatcgtttttacttctaggccttcacctagctggggtgagtggccagcaggagaaacgtgaccagcagcaggtgagacaaagtccccaatctctgacagtctgggaaggagagaccgcaattctgaactgcagttatgagaacagtgcttttgactacttcccatggtaccagcagttccctggggaaggtcccgctctcctgatatccatactttcagtgtccaataaaaaggaagatggacgattcacaatcttcttcaataaaagggagaaaaagctctccttgcacattgcagactctcagcctggagactcagccacctacttctgtgcagggggagactatgcaaacaagatgatctttggcttgggaaccattttgagagtcagacctcacatccagaacccagaacctgctgtgtaccagctgaaagatccgcgcagccaggatagcaccctgtgcctgtttaccgattttgatagccagattaacgtgccgaaaaccatggaaagcggcacctttattaccgataaaaccgtgctggatatgaaagcgatggatagcaaaagcaacggcgcgattgcgtggagcaaccagaccagctttacctgccaggatatttttaaagaaaccaacgcgacctatccgagcagcgatgtgccgtgcgatgcgaccctgaccgaaaaaagctttgaaaccgatatgaacctgaactttcagaacctgagcgtgatgggcctgcgcattctgctgctgaaagtggcgggctttaacctgctgatgaccctgcgcctgtggagcagc

SEQ ID NO: 2 - protein, 275 aa, synthetic construct
  REGION 1..275: note = TCR alpha chain, amino acid
MDKILTASFLLLGLHLAGVSGQQEKRDQQQVRQSPQSLTVWEGETAILNCSYENSAFDYFPWYQQFPGEGPALLISILSVSNKKEDGRFTIFFNKREKKLSLHIADSQPGDSATYFCAGGDYANKMIFGLGTILRVRPHIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 3 - DNA, 918 bp, synthetic construct
  misc_feature 1..918: note = TCR beta chain, DNA
  source 1..918: mol_type = other DNA
atgggctccattttcctcagttgcctggccgtttgtctcctggtggcaggtccagtcgacccgaaaattatccagaaaccaaaatatctggtggcagtcacagggagcgaaaaaatcctgatatgcgaacagtatctaggccacaatgctatgtattggtatagacaaagtgctaagaagcctctagagttcatgttttcctacagctatcaaaaacttatggacaatcagactgcctcaagtcgcttccaacctcaaagttcaaagaaaaaccatttagaccttcagatcacagctctaaagcctgatgactcggccacatacttctgtgccagcagccaggacaggtcctataattcgcccctctactttgcggcaggcacccggctcactgtgacagaggatctgagaaatgtgactccacccaaggtgagcctgtttgaaccgagcaaagcggaaattgcgaacaaacagaaagcgaccctggtgtgcctggcgcgcggcttttttccggatcatgtggaactgagctggtgggtgaacggcaaagaagtgcatagcggcgtgagcaccgatccgcaggcgtataaagaaagcaactatagctattgcctgagcagccgcctgcgcgtgagcgcgaccttttggcataacccgcgcaaccattttcgctgccaggtgcagtttcatggcctgagcgaagaagataaatggccggaaggcagcccgaaaccggtgacccagaacattagcgcggaagcgtggggccgcgcggattgcggcattaccagcgcgagctatcagcagggcgtgctgagcgcgaccattctgtatgaaattctgctgggcaaagcgaccctgtatgcggtgctggtgagcaccctggtggtgatggcgatggtgaaacgcaaaaacagc

SEQ ID NO: 4 - protein, 306 aa, synthetic construct
  REGION 1..306: note = TCR beta chain, amino acid
MGSIFLSCLAVCLLVAGPVDPKIIQKPKYLVAVTGSEKILICEQYLGHNAMYWYRQSAKKPLEFMFSYSYQKLMDNQTASSRFQPQSSKKNHLDLQITALKPDDSATYFCASSQDRSYNSPLYFAAGTRLTVTEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 5 - protein, 120 aa, synthetic construct
  REGION 1..120: note = TCR alpha chain variable region, amino acid
MDKILTASFLLLGLHLAGVSGQQEKRDQQQVRQSPQSLTVWEGETAILNCSYENSAFDYFPWYQQFPGEGPALLISILSVSNKKEDGRFTIFFNKREKKLSLHIADSQPGDSATYFCAGG

SEQ ID NO: 6 - protein, 19 aa, synthetic construct
  REGION 1..19: note = TCR alpha chain junction region, amino acid
DYANKMIFGLGTILRVRPH

SEQ ID NO: 7 - protein, 136 aa, synthetic construct
  REGION 1..136: note = TCR alpha chain constant region, amino acid
IQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 8 - protein, 114 aa, synthetic construct
  REGION 1..114: note = TCR beta chain variable region, amino acid
MGSIFLSCLAVCLLVAGPVDPKIIQKPKYLVAVTGSEKILICEQYLGHNAMYWYRQSAKKPLEFMFSYSYQKLMDNQTASSRFQPQSSKKNHLDLQITALKPDDSATYFCASSQ

SEQ ID NO: 9 - skipped

SEQ ID NO: 10 - protein, 16 aa, synthetic construct
  REGION 1..16: note = TCR beta chain junction region, amino acid
YNSPLYFAAGTRLTVT

SEQ ID NO: 11 - protein, 173 aa, synthetic construct
  REGION 1..173: note = TCR beta constant region, amino acid
EDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 12 - protein, 6 aa, synthetic construct
  REGION 1..6: note = TCR alpha chain CDR-1
NSAFDY

SEQ ID NO: 13 - protein, 7 aa, synthetic construct
  REGION 1..7: note = TCR alpha chain CDR-2
ILSVSNK

SEQ ID NO: 14 - skipped

SEQ ID NO: 15 - protein, 5 aa, synthetic construct
  REGION 1..5: note = TCR beta chain CDR-1
LGHNA

SEQ ID NO: 16 - protein, 6 aa, synthetic construct
  REGION 1..6: note = TCR beta chain CDR-2
YSYQKL

SEQ ID NO: 17 - protein, 4 aa, synthetic construct
  REGION 1..4: note = TCR beta chain CDR-3
ASSQ

SEQ ID NO: 18 - DNA, 23 bp, synthetic construct
  misc_feature 1..23: note = TCR alpha (TRAC) targeting gRNA (mouse)
  source 1..23: mol_type = other DNA
gtgccgaaaaccatggaatctgg

SEQ ID NO: 19 - DNA, 23 bp, synthetic construct
  misc_feature 1..23: note = TCR alpha (TRAC) targeting gRNA (mouse)
  source 1..23: mol_type = other DNA
aatcaatgtgccgaaaaccatgg

SEQ ID NO: 20 - DNA, 23 bp, synthetic construct
  misc_feature 1..23: note = TCR beta (TRBC) targeting gRNA (mouse)
  source 1..23: mol_type = other DNA
gcagttccatgggctttcagagg

SEQ ID NO: 21 - DNA, 23 bp, synthetic construct
  misc_feature 1..23: note = TCR beta (TRBC) targeting gRNA (mouse)
  source 1..23: mol_type = other DNA
tggggtcagcacggaccctcagg

SEQ ID NO: 22 - DNA, 23 bp, synthetic construct
  misc_feature 1..23: note = TCR alpha (TRAC) targeting gRNA (human)
  source 1..23: mol_type = other DNA
tgtgctagacatgaggtctatgg

SEQ ID NO: 23 - DNA, 23 bp, synthetic construct
  misc_feature 1..23: note = TCR beta (TRBC) targeting gRNA (human)
  source 1..23: mol_type = other DNA
ggagaatgacgagtggacccagg